(19) United States
(12) Patent Application Publication
(10) Pub. No.: US 2012/0093885 A1
(43) Pub. Date: Apr. 19, 2012

(54) THERAPEUTIC VESICLES

(75) Inventors: Susmita Sahoo, Chicago, IL (US); Douglas W. Losordo, Chicago, IL (US); David E. Zembower, La Grange, IL (US)

(73) Assignee: NORTHWESTERN UNIVERSITY, Evanston, IL (US)

(21) Appl. No.: 13/276,142

(22) Filed: Oct. 18, 2011

Related U.S. Application Data
(60) Provisional application No. 61/394,193, filed on Oct. 18, 2010.

(51) Int. Cl. A61K 35/12; A61P 9/00; A61P 9/10; A61P 1/04; A61P 7/02; A61P 9/04; A61P 19/00; A61P 25/28; A61P 3/10; A61P 25/16; A61P 17/02; A61P 25/00; A61P 7/00; C12N 5/074; A61K 9/00

(52) U.S. Cl. 424/400; 424/520; 435/325; 435/405

(57) ABSTRACT

The present technology provides compositions of vesicles, uses of vesicles, and methods relating to vesicles. For example, provided herein are vesicles derived from stem cells for use in regenerative therapies.

### CROSS-REFERENCE TO RELATED APPLICATION

The present invention claims priority to U.S. Provisional Patent Application Ser. No. 61/394,193 filed Oct. 18, 2010, which is hereby incorporated by reference in its entirety.

### STATEMENT REGARDING FEDERALLY SPONSORED RESEARCH OR DEVELOPMENT

This invention was made with government support under Grant No. RO1 HL053354 awarded by the National Institutes of Health. The government has certain rights in the invention.

### FIELD OF INVENTION

Provided herein are compositions of vesicles, uses of vesicles, and methods relating to vesicles. For example, provided herein are vesicles derived from stem cells for use in regenerative therapies.

### BACKGROUND

Cardiovascular disease is the leading cause of death in the Western world. In the United States, 71 million Americans are affected by cardiovascular disease with the associated costs of treatment approximated to be $400 billion. In cases where disease is caused by poor vascularization or insufficient blood supply, production of new blood vessels can be an effective therapy. Some current modes of angiogenic therapy include cell-based therapies, gene therapy, and protein therapy. Despite their promise, these therapies remain problematic. Cell-based therapies are still in early stages of research, with many open questions regarding the best cell types to use and concerns about the complexity of cells and their potential to induce undesired side effects. Foremost amongst the problems with cell-based therapies are immunological incompatibility and practical considerations such as the difficulty of isolating adequate numbers of cells. Furthermore, gene therapy requires effective integration of therapeutic genes into target cell genomes and has the risks of inducing undesired immune responses, potential toxicity, immunogenicity, inflammation, and oncogenesis. Delivery presents an obstacle for protein therapies because routes of protein administration do not prevent proteins from being processed or cleared before entering the target tissue. Accordingly, angiogenic treatment of cardiovascular diseases requires the development of new modes of therapy that minimize or eliminate these and other problems.

### SUMMARY

Provided herein are compositions of vesicles, uses of vesicles, and methods relating to vesicles. For example, provided herein are vesicles derived from stem cells for use in regenerative therapies.

In some embodiments, the compositions and methods herein provide therapies wherein vesicles derived from adult stem cells are used to regenerate damaged tissue. One important type of such a regenerative therapy is angiogenic therapy, which can reverse the tissue damage associated with cardiovascular disease. Tissue damage frequently accompanies cardiovascular disease because poor blood flow can cause starvation and subsequent deterioration of various tissues throughout the body. Accordingly, forming new blood vessels to supply oxygen and required nutrients to damaged tissues can promote healing and regeneration of the damaged tissue. Importantly, while adult stem cells have shown promise in regenerative therapies, it is provided herein that vesicles derived from adult stem cells perform similar therapeutic functions more safely and more effectively. In some tests, stem cell-derived vesicles were one hundred times more effective than the cells from which the vesicles were prepared. In addition, the vesicle compositions described herein can be prepared in vitro and can be stored (e.g., frozen) for later use, and the methods described herein involve administering a minimal volume and mass of therapeutic agent to subjects requiring treatment. Consequently, because stem cell-derived vesicles possess many practical and technical advantages relative to stem cells, the therapies described herein are important developments in the field of regenerative medicine.

In one embodiment, provided herein is a method comprising administering to a subject a therapeutically effective amount of purified adult stem cell vesicles or an adult stem cell vesicle extract. In some embodiments, the vesicles are exosomes. The vesicles or exosomes may contain various cell-derived components such as protein, DNA, or RNA (e.g., a miRNA). In some embodiments the included proteins are characteristic of exosomes. For example, in some embodiments the vesicles contain TSG101 and CD63 proteins and in other embodiments the vesicles contain CD34+ protein. Moreover, some embodiments provide a composition (e.g., vesicles, exosomes, an extract) comprising at least two purified molecules selected from the group consisting of miRNA 130a, miRNA 125b, miRNA 92a, miRNA 126, haptoglobin, and hemopexin. Some embodiments provide that the composition comprises at least three, at least four, at least five, or at least six molecules selected from the group consisting of miRNA 130a, miRNA 125b, miRNA 92a, miRNA 126, haptoglobin, and hemopexin.

Importantly, the methods are not limited to the source of the stem cells. In various embodiments, the sources of stem cells include, but are not limited to, cord blood, bone marrow, peripheral blood, brain, spinal cord, blood vessels, skeletal muscle, skin, teeth, heart, gut, liver, ovarian epithelium, amniotic fluid, umbilical cord, or testis. Furthermore, the methods are not limited in the modes of administering the therapy. Embodiments include, but are not limited to, administration by injection catheter, by intramyocardial injection, by intracoronary administration, by intracoronary infusion, by an intravenous injection, or by nanoparticles. In addition, the scope of subjects who could benefit from the methods is not limited. In some embodiments, the subject requires angiogenic therapy. In other embodiments, the subject's disease state includes, but is not limited to, cardiovascular disease, infarction, chronic wounds, ulcer, clogged vessels, damaged vessels, stenotic vessels, atherosclerosis, angina, peripheral vascular disease, critical limb ischemia, ischemic heart disease, hypoxic tissues, heart failure, bone marrow disease, Alzheimer's disease, diabetes, or Parkinson's disease. In some embodiments, the subject requires wound healing, scar reduction, or tissue regeneration. In some embodiments, the subject has a bone marrow transplant, or has tissue damage from a stroke, hemorrhage, thrombosis, embolism, or hypoperfusion.

Another embodiment provided herein is a composition comprising purified and isolated adult stem cell vesicles or an adult stem cell vesicle extract. Vesicles prepared from different cell types can possess different characteristics. While there is no limitation on the types of vesicles provided, in one embodiment the vesicles are exosomes. Furthermore, while there is no limitation on the physical characteristics of the vesicles, in one embodiment the vesicles are cup shaped, are 30-100 nm in diameter, or have a density of 1.1-1.2 g/cm³. The vesicles may contain many different biological components, including, but not limited to, protein, lipids, DNA, RNA, cofactors, salts, amino acids, and nucleotides. For example, some embodiments provide a composition comprising at least two purified molecules selected from the group consisting of miRNA 130a, miRNA 125b, miRNA 92a, miRNA 126, haptoglobin, and hemopexin. Some embodiments provide that the composition comprises at least three, at least four, at least five, or at least six molecules selected from the group consisting of miRNA 130a, miRNA 125b, miRNA 92a, miRNA 126, haptoglobin, and hemopexin. Furthermore, some components such as proteins may be present in the lumen of the vesicle or embedded in the membrane. In some embodiments, the vesicles contain TSG101 and CD63 proteins. In other embodiments, the vesicles contain CD34 protein. The vesicles may be derived from cells of the subject or from another individual; thus, in some embodiments the vesicles are derived from an autologous source and in other embodiments the vesicles are derived from an allogeneic source. In some embodiments, the vesicles are derived from an autologous source by a method comprising mobilizing CD34+ cells by treating the autologous source with a mobilizing agent; enriching the CD34+ cells using apheresis; and further enriching the CD34+ cells using a magnetic bead cell selection device. In some embodiments, the mobilizing agent is GCSF or AMD3100. Thus, in some embodiments, the CD+ cells are derived from a GCSF- or AMD3100-mobilized source of animal adult stem cells.

Some embodiments of the technology provide a therapeutically effective amount of a composition comprising purified and isolated adult stem cell vesicles or an adult stem cell vesicle extract. In some embodiments, the composition comprises at least 10⁴, at least 10⁵, at least 10⁶, at least 10⁷, at least 10⁸, or more vesicles. For example, in some embodiments, compositions comprise 10⁴ to 10⁹ vesicles (e.g., the compositions comprise 10⁴ to 10⁵ vesicles, 10⁵ to 10⁶ vesicles, 10⁶ to 10⁷ vesicles, 10⁷ to 10⁸ vesicles, or 10⁸ to 10⁹ vesicles). In some embodiments, the amount of vesicles in the composition is 0.1 or more gram (e.g., 0.1 to 1.0 gram). In some embodiments, the amount of vesicles in the composition is 1.0 or more gram (e.g., 1.0 to 10.0 grams). In some embodiments, the amount of the vesicles in the composition is 10.0 or more grams (e.g., 10.0 to 100.0 grams). In some embodiments, the vesicles are from 10³ or more stem cells (e.g., approximately 10³ to 10⁴ stem cells); in some embodiments, the vesicles are from 10⁴ or more stem cells (e.g., approximately 10⁴ to 10⁵ stem cells); in some embodiments, the vesicles are from 10⁵ or more stem cells (e.g., approximately 10⁵ to 10⁶ stem cells); in some embodiments, the vesicles are from 10⁶ or more stem cells (e.g., approximately 10⁶ to 10⁷ stem cells); in some embodiments, the vesicles are from 10⁷ or more stem cells (e.g., approximately 10⁷ to 10⁸ stem cells); in some embodiments, the vesicles are from 10⁸ or more stem cells (e.g., approximately 10⁸ to 10⁹ stem cells).

In some embodiments, the extract is from 10³ or more stem cells (e.g., approximately 10³ to 10⁴ stem cells); in some embodiments, the extract is from 10⁴ or more stem cells (e.g., approximately 10⁴ to 10⁵ stem cells); in some embodiments, the extract is from 10⁵ or more stem cells (e.g., approximately 10⁵ to 10⁶ stem cells); in some embodiments, the extract is from 10⁶ or more stem cells (e.g., approximately 10⁶ to 10⁷ stem cells); in some embodiments, the extract is from 10⁷ or more stem cells (e.g., approximately 10⁷ to 10⁸ stem cells); in some embodiments, the extract is from 10⁸ or more stem cells (e.g., approximately 10⁸ to 10⁹ stem cells).

Some embodiments provide methods of preparing vesicles comprising, e.g., culturing adult stem cells in conditioned media, isolating the cells from the conditioned media, purifying the vesicles (e.g., by sequential centrifugation), and, optionally, clarifying the vesicles on a density gradient. In some embodiments, the vesicles are essentially free of non-vesicle stem cell components. The embodiments are not limited with respect to the types or sources of cells that can be used. For example, in one embodiment, the cells are CD34+ cells. In a more specific embodiment, the CD34+ cells are derived from a GCSF-mobilized source of animal adult stem cells or from an AMD3100-mobilized source of animal adult stem cells. Additionally, in one embodiment, the source of animal adult stem cells is peripheral blood. The embodiments are not limited in the types of media that can be used to culture the cells. In one embodiment, the conditioned media is supplemented with human serum albumin (e.g., 0.1-5.0%; e.g., 1.0%), FLT ligand (e.g., 50-150 ng/ml), SCF (e.g., 50-150 ng/ml), or VEGF (e.g., 1-50 ng/ml). In some embodiments of the methods provided herein, the vesicles are separated from cells, e.g., by using sequential centrifugation. In one embodiment, the sequential centrifugation comprises centrifuging at about 400-500×g (e.g., 400×g for 10 minutes), then centrifuging at about 1800-2200×g (e.g., 2000×g for 10 minutes), and centrifuging at about 18,000-22,000×g (e.g., 20,000×g for 20 minutes), followed by pelleting the vesicles by centrifugation (e.g., at 120,000×g for 60 minutes).

In some embodiments, cells and conditioned media are separated, e.g., by centrifugation at about 500-1000×g (e.g., 800×g for 5 minutes), the conditioned media is clarified, e.g., by centrifugation at about 10,000-20,000×g (e.g., 14,000×g for 20 minutes), and the exosomes are collected, e.g., by ultracentrifugation (e.g., at 100,00×g for 60 minutes on a 25-35% sucrose-D₂O solution having a density of ˜1.0-1.2 g/cm³ (e.g., about 1.127 g/cm³)). Following a wash (e.g., in PBS) the exosomes are pelleted and re-suspended (e.g., in PBS) for use. While there is no limitation on the temperature at which the centrifugation may be performed, one embodiment provides for centrifugation to be performed at about 0-10° C. (e.g., 4° C.). In other embodiments, the vesicles are clarified, e.g., by separation on a density gradient. In some embodiments, sucrose is used to form the density gradient. For example, some embodiments provide for floating the vesicles on a 25-35% sucrose density gradient, washing and pelleting the vesicles (e.g., in PBS), and resuspending the vesicles (e.g., in 0.22 μm-filtered PBS with 0.01-1% human serum albumin). An advantage of the methods provided herein is that the vesicles can be stored for future use. As an example of this advantage, one embodiment includes freezing the vesicles (e.g., at −80° C.).

Some embodiments provide for use of a composition comprising purified and isolated vesicles or an extract prepared from animal adult stem cells for a medicament. Other embodiments provided herein are for use of a composition comprising purified and isolated vesicles or an extract prepared from animal adult stem cells for the manufacture of a medicament. The medicament is not limited to particular uses. As an example of one embodiment, the medicament is used for regenerative therapy. In a more specific example of an embodiment, the regenerative therapy is angiogenic therapy. In other embodiments, the medicament is used to treat diseases including, but not limited to, cardiovascular disease, infarction, chronic wounds, ulcer, clogged vessels, damaged vessels, stenotic vessels, atherosclerosis, angina, peripheral vascular disease, critical limb ischemia, ischemic heart disease, hypoxic tissues, heart failure, bone marrow disease, Alzheimer's disease, diabetes, or Parkinson's disease. Additional embodiments provide for use of the medicament in diseases that involve wound healing, scar reduction, or tissue regeneration; in disease that involves a bone marrow transplant; and in disease that involves tissue damage from stroke, hemorrhage, thrombosis, embolism, or hypoperfusion.

These and other features, aspects, and advantages of the present technology will become better understood with reference to the following description and claims.

### DESCRIPTION OF THE DRAWINGS

These and other features, aspects, and advantages of the present technology will become better understood with regard to the following drawings:

FIG. 1 shows electron micrographs of isolated exosomes from CD34+ cells and MNCs showing cup-shaped morphology. FIG. 1a is a transmission electron micrograph of CD34+ cell (i) cytoplasm with MVBs enclosing numerous bilipidic layer-bound exosomes (Exo) (inset, arrows), (ii) inward invagination (arrows) in the MVB membrane indicate the beginnings of exosome biogenesis, (iii) MVB fusing with cell membrane, (iv) Exosomes are secreted out from the cell. FIG. 1b shows micrographs of exosomes purified from CD34+ cells and MNC CM.

FIG. 2 shows plots of data from dynamic light scattering experiments for exosomes isolated from CD34+ cells and MNCs. The distributions demonstrate a single peak (˜40-90 nm diameter) indicating that the preparations are free of contamination.

FIG. 3 shows flow cytometry dot plots resulting from analysis of exosomes from human CD34+ cells and MNCs. FIG. 2a demonstrates detection of the exosomal surface protein CD63 and FIG. 2b demonstrates Annexin V bound to exposed phosphatidylserine.

FIG. 4 shows flow cytometry dot plot analysis for the CD34+ surface protein. FIG. 4a shows the results from experiments in which isolated exosomes were conjugated to 4-μm latex beads and stained. The numbers inside the boxes indicate the percentage of positive beads counted. FIG. 4b shows dot plots of isolated exosomes from MNCs or CD34+ cells stained with FITC-conjugated CD34+ antibody or an isotype control, followed by staining with cellvue maroon dye. Numbers inside the boxes indicate the percentage of positive exosomes. The histogram shows the spectral shift for stained CD34+ exosomes as compared to the isotype control and stained MNC exosomes.

FIG. 5 shows an immunoblot for exosomal intraluminal exosomal protein, TSG101, from both CD34+ exosomes and MNC exosomes.

FIG. 6 shows plots of data from dynamic light scattering analyses of CD34+ conditioned media, CD34+ exosomes, and exosome depleted-CM demonstrating the isolation of exosomes without protein and other contaminating debris from the conditioned media.

FIG. 7a shows a plot of data from in vitro experiments to test the induction of Matrigel tube formation in HUVECs by incubation with CD34+ exosomes for 8 hours. FIG. 7b shows a plot of data from a dose-response experiment to test CD34+ exosome-induced tube formation in HUVECs. FIG. 7c shows a plot of data from experiments to test the viability of HUVECs in the presence of CD34+ exosomes. FIG. 7d shows a plot of data from experiments to test the proliferation of HUVECs in the presence of CD34+ exosomes. n=3-6; *P<0.001 versus PBS, †P<0.05 versus Exo-depleted CM, ‡P<0.05 versus MNCs or MNC exosomes.

FIG. 8 shows a plot of data from in vitro experiments to test the induction of tube formation in HUVECs by incubation for 8 hours with exosomes prepared from MNCs. n=3-4. Tube length is expressed as a percentage of the length measured for PBS-treated HUVECs.

FIG. 9 shows a plot of data from in vitro experiments to test the induction of tube formation in HUVECs by incubation with CD34+ or MNC preparations of cells, conditioned media (CM), exosomes (Exo), or exosome-depleted conditioned media for 24 hours. Tube length is expressed as a percentage of the length measured for PBS-treated HUVECs. n=3-6. *P<0.005 versus PBS, †P<0.05 versus MNCs or MNC exosomes.

FIG. 10 is shows data from in vivo Matrigel experiments to test the induction of vessel growth by CD34+ exosomes. FIG. 10a shows the vessel-like structures formed in the Matrigel following treatment with CD34+ exosomes. FIG. 10b shows data quantifying the CD31+ mouse endothelial cells in the Matrigel. n=3. *P<0.05 versus PBS.

FIG. 11a is an electron micrograph from an in vivo corneal implant assay showing vessel growth induced by CD34+ exosomes. FIG. 11b is a plot of data showing the extent of vessel growth in the cornea treated with CD34+ exosomes. n=4. *P<0.05 versus PBS, ‡P<0.01 versus MNC exosomes.

FIG. 12 is a series of photographs from in vivo experiments to test the recovery of an ischemic limb from amputation by treatment with CD34+ exosomes.

FIG. 13 shows plots of data resulting from experiments to test the functional recovery of an ischemic limb after the induction of limb perfusion by treatment with CD34+ exosomes. Data are presented as the ratio of perfusion in ischemic to non-ischemic limbs at different time points; the mean ratio of all mice in each group is used for each data point. n=7-12 per group. *P<0.05 versus the PBS and MNC Exo group.

FIG. 14 shows plots of data from experiments to test the functional recovery of an ischemic limb by treatment with CD34+ exosomes. FIG. 14a shows that CD34+ exosomes improve the limb motor score of the ischemic limb and FIG. 14b shows that CD34+ exosomes improve the limb salvage score of the ischemic limb. Limb motor scores are as follows—1: no limb use; 2: no foot use, limb use only; 3: restricted foot use; 4: no active toe use (spreading), foot use only; and 5: unrestricted limb use. Limb salvage (i.e. no tissue necrosis) scores are as follows-1: limb amputation; 2: foot amputation; 3: toe(s) amputation; 4: necrosis, nail loss only; 5: full recovery. n=7-12 per group. *P<0.05.

FIG. 15a is a series of electron micrographs from in vivo experiments to test the induction of capillary formation in the mouse hind limb ischemia model. FIG. 15b shows plots of data representing the ratio of capillary density between the ischemic and non-ischemic limb for the indicated category of treatment. *P<0.05.

FIG. 16 shows a gel from a two-dimensional (2-D) differential gel electrophoresis (DIGE) experiment that demonstrates protein enrichment in CD34+ exosomes. The numbered proteins are identified in Table 1.

FIG. 17 shows plots of data from experiments to quantify and test the quality of RNA prepared from exosomes. FIG. 17a is a plot of data showing the mass in nanograms of RNA recovered, FIG. 17b is a data plot showing the ratio of absorbances at 260 nm and 280 nm as a measure of RNA quality, and FIG. 17c is a data plot showing the ratio of absorbances at 260 nm and 230 nm as a second measure of RNA quality.

FIG. 18 shows data plots that resulted from analysis of RNA preparations for size, quantity, and quality by Agilent Bioanalyzer. “Total RNA Chip” shows the results of analysis of total RNA and “Small RNA Chip” shows the results of analysis of small RNA.

FIG. 19 shows plots of data from experiments showing that RNA isolated with exosomes is contained within the lumen of the exosomes.

FIG. 20 shows plots of data from experiments comparing the expression of miRNA 126 in different samples. n=3; fold change, CD34+ Exo:MNC Exo=50 fold, P=0.07 (for has-miRNA-126).

FIG. 21 shows plots of data from experiments comparing the expression of miRNA 130a in different samples. n=3; fold change, CD34+ Exo:MNC Exo=50 fold, P=0.04 (for has-miRNA-130a).

FIG. 22 shows plots of data from experiments comparing the expression of miRNA 125b in different samples. n=3; fold change, CD34+ Exo:MNC Exo=180 fold, P=0.001 (for has-miRNA-125b).

FIG. 23 shows plots of data from experiments comparing the expression of miRNA 92a in different samples. n=3; fold change, CD34+ Exo:MNC Exo=5 fold, p=0.0008 (for has-miRNA-92a).

FIG. 24 shows plots of data from experiments measuring the expression of representative pro-angiogenic miRNAs in CD34+ cells and exosomes by RT-PCR.

FIG. 25 shows plots of data from experiments showing that CD34+ exosomes transfer pro-angiogenic miRNA to MNCs.

FIG. 26 shows plots of data from flow cytometry experiments showing that HUVECs take up CD34+ exosomes.

FIG. 27a shows plots of data from flow cytometry experiments showing that Cy3 miRNA is present in CD34+ exosomes. FIG. 27b shows confocal microscopy images demonstrating that Cy3 miRNA in CD34+ exosomes is transferred to human umblical vein endothelial cells.

FIG. 28 shows a plot of data showing that cord blood derived CD34+ exosomes increase tube formation of human umbilical vein endothelial cells.

### DETAILED DESCRIPTION

Provided herein are compositions of vesicles, uses of vesicles, and methods relating to vesicles. For example, provided herein are vesicles derived from stem cells for use in regenerative therapies. For example, in some embodiments, provided herein are compositions comprising exosomes derived from CD34+ adult stem cells or other adult stem cells, methods of using said exosomes for therapeutic angiogenesis and regeneration of tissue that has been damaged by ischemia, and methods of preparing said exosomes.

Exosomes (also known as “nano-vesicles”) are released from cells as a component of cellular paracrine secretions. They are double membrane-bound cup-shaped vesicles of approximately 30-100 nm in diameter (see, e.g., Théry, C. F1000 Biol Rep. 2011, 3: 15). Exosomes originate intracellularly in multivesicular bodies (MVB) and are secreted when the MVBs fuse with the plasma membrane (Chaput N. and Théry C. Semin Immunopathol. 2011, 33(5): 419-40). They contain trans-membrane proteins and enclose soluble hydrophilic components such as nucleic acids and proteins derived from the cytoplasm of the cell of origin. These nucleic acid molecules, particularly RNAs and microRNAs (miRNA), can be taken up and transcribed by the target recipient cells and modulate cell physiology (Mittelbrunn et al, Nat Commun, 2011, 2: 282; Valadi et al, Nat Cell Biol, 2007, 6: 654). Exosomes are secreted by CD34+ cells (Sahoo S. et al., Circ Res. 2011, 109(7): 724-8) and they mediate at least a part of the CD34+ cell therapeutic function such as functional recovery and angiogenesis in ischemic tissues. Accordingly, CD34+ exosomes are a suitable cell-free alternative to stem cell transplantation. Unlike cells, which have a function that depends on their viability in the ischemic environment, use of exosomes provides a more efficacious and convenient cell-free alternative to CD34+ cell transplantation for tissue repair and regeneration.

### DEFINITIONS

In order that the present technology may be more readily understood, certain terms are first defined. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. Thus, “a” or “an” or “the” can mean one or more than one. For example, “a” widget can mean one widget or a plurality of widgets. The meaning of “in” includes “in” and “on.”

As used herein, the term “ischemia” refers to any localized tissue ischemia due to reduction of the inflow or outflow of blood.

As used herein, the term “angiogenesis” refers to the process by which new blood vessels are generated from existing vasculature and tissue. The phrase “repair or remodeling” refers to the reformation of existing vasculature. The spontaneous growth of new blood vessels provides collateral circulation in and around an ischemic area, improves blood flow, and alleviates the symptoms caused by the ischemia. Angiogenesis-mediated diseases and disorders include acute myocardial infarction, ischemic cardiomyopathy, peripheral vascular disease, ischemic stroke, acute tubular necrosis, ischemic wounds, sepsis, ischemic bowel disease, diabetic retinopathy, neuropathy and nephropathy, vasculitidies, ischemic encephalopathy, erectile dysfunction, ischemic or traumatic spinal cord injuries, multiple organ system failure, ischemic gum disease, and transplant-related ischemia.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein the term “disease” refers to a deviation from the condition regarded as normal or average for members of a species, and which is detrimental to an affected individual under conditions that are not inimical to the majority of individuals of that species (e.g., diarrhea, nausea, fever, pain, inflammation, etc.).

As used herein, “stem cell” refers to a multipotent cell with the potential to differentiate into a variety of other cell types (which perform one or more specific functions), and have the ability to self-renew. As used herein, “adult stem cells” refer to stem cells that are not embryonic stem cells.

As used herein, the terms “administering”, “introducing”, “delivering”, “placement” and “transplanting” are used interchangeably and refer to the placement of the vesicles, liposomes, or exosomes of the technology into a subject by a method or route that results in at least partial localization of the vesicles, liposomes, or exosomes at a desired site. The vesicles, liposomes, or exosomes can be administered by any appropriate route that results in delivery to a desired location in the subject where at least a portion of the vesicles, liposomes, or exosomes or components of the vesicles, liposomes, or exosomes retain their therapeutic capabilities.

As used herein, the term “treating” includes reducing or alleviating at least one adverse effect or symptom of a disease or disorder through introducing in any way a therapeutic composition of the present technology into or onto the body of a subject.

As used herein, “therapeutically effective dose” refers to an amount of a therapeutic agent sufficient to bring about a beneficial or desired clinical effect. Said dose could be administered in one or more administrations. However, the precise determination of what would be considered an effective dose may be based on factors individual to each patient, including, but not limited to, the patient's age, size, type or extent of disease, stage of the disease, route of administration, the type or extent of supplemental therapy used, ongoing disease process, and type of treatment desired (e.g., aggressive vs. conventional treatment).

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “pharmaceutical composition” refers to the combination of an active agent with, as desired, a carrier, inert or active, making the composition especially suitable for diagnostic or therapeutic use in vitro, in vivo, or ex vivo.

As used herein, the terms “pharmaceutically acceptable” or “pharmacologically acceptable” refer to compositions that do not substantially produce adverse reactions, e.g., toxic, allergic, or immunological reactions, when administered to a subject.

As used herein, the terms “host”, “patient”, or “subject” refer to organisms to be treated by the compositions of the present technology or to be subject to various tests provided by the technology. The term “subject” includes animals, preferably mammals, including humans. In a preferred embodiment, the subject is a primate. In an even more preferred embodiment, the subject is a human.

As used herein, the term “purified” or “to purify” refers to the removal of contaminants or undesired compounds from a sample or composition. As used herein, the term “substantially purified” refers to the removal of from about 70 to 90%, up to 100%, of the contaminants or undesired compounds from a sample or composition. In certain embodiments, 95%, 96%, 97%, 98%, 99%, or 99.5% of non-vesicle components are removed from a preparation.

As used herein, the term “sample” is used in its broadest sense. In one sense it can refer to animal cells or tissues. In another sense, it is meant to include a specimen or culture obtained from any source, such as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

As used herein, “wound healing” is intended to include all disorders characterized by any disease, disorder, syndrome, anomaly, pathology, or abnormal condition of the skin and/or underlying connective tissue, e.g., skin wounds following surgery, skin abrasions caused by mechanical trauma, caustic agents or burns, cornea following cataract surgery or corneal transplants, mucosal epithelium wounds following infection or drug therapy (e.g., respiratory, gastrointestinal, genitourinary, mammary, oral cavity, ocular tissue, liver and kidney), diabetic wounds, skin wounds following grafting, and regrowth of blood vessels following angioplasty. Treatment of a wound, disease or disorder is within the gambit of regenerative medicine.

### Embodiments of the Technology

Although the disclosure herein refers to certain illustrated embodiments, it is to be understood that these embodiments are presented by way of example and not by way of limitation.

The production of new blood vessels is an effective therapy for ischemic diseases (e.g., myocardial ischemia and critical limb ischemia) caused by poor vascularization or insufficient blood supply. As demonstrated during the development of embodiments of the technology provided herein, exosomes compose the major pro-angiogenic component of human CD34+ cell paracrine secretions and induce angiogenesis similarly to CD34+ cells.

### Exosomes

Exosomes are vesicles formed via a specific intracellular pathway involving multivesicular bodies or endosomal-related regions of the plasma membrane. They generally have a discrete size of approximately 30-90 nm, a characteristic buoyant density of approximately 1.1-1.2 g/ml, and a characteristic lipid composition. Exosomes express certain marker proteins, but generally lack markers of lysosomes, mitochondria, or caveolae (Théry et al, Curr Prot Cell Biol, 2006, 3: 3.22). Exosomes typically also express specific cell-surface proteins including integrins and cell adhesion molecules (Clayton et al, FASEB J, 2004, 9:977), so they have the means to bind selectively to, and be taken up by, specific recipient cell types (Lasser et al, J Transl Med, 2011, 9: 9; Tian et al, J Cell Biochem, 2010 111(2): 488; Feng et al, Traffic, 2010, 5:675).

As demonstrated by experiments conducted during the development of embodiments described herein, human adult CD34+ cells secrete exosomes that mediate at least a part of stem cells' therapeutic function.

A composition prepared by isolating exosomes from human adult CD34+ stem cells promotes the regeneration of damaged tissues by stimulating neovascularization. As a regenerative therapy, administering the stem cell-derived exosome composition to damaged tissues speeds healing by increasing the delivery of oxygen and other nutrients to damaged tissue.

An exemplary method of producing exosomes comprises culturing adult stem cells in conditioned media, isolating the cells from the conditioned media, purifying the vesicles by sequential centrifugation, and clarifying the vesicles on a density gradient. In some embodiments, exosomes are prepared from GCSF-mobilized adult human peripheral blood CD34+ cells (Losordo et al, Circ Res, 2011, 109(4): 428) as follows: The CD34+ cells are cultured in media supplemented with 1% human serum albumin, 100 ng/ml of FLT-ligand, 100 ng/ml of SCF, and 10 ng/ml VEGF. Exosomes devoid of contaminating cell debris and other vesicles are obtained by sequential centrifugation, for example, at 400×g for 10 minutes, 2000×g for 10 minutes, and 20,000×g for 20 minutes at 4° C. The exosomes are pelleted from the conditioned media by centrifuging, for example, at 120,000×g for 60 minutes at 4° C. Ultrapure exosomes are collected by floating the exosomes on a 30% sucrose density gradient for 60 minutes at 4° C., followed by washing and pelleting the exosomes in PBS. The exosomes are resuspended in 0.22 μm-filtered PBS with 0.1% human serum albumin. In some embodiments, the exosomes prepared this way can be stored frozen, e.g., at −80° C., without significant loss of potency, e.g., when thawed for use.

For the development of some embodiments described herein, experiments used peripheral blood (PB) CD34+ cells purified from PB-derived total mononuclear cells of healthy volunteers. Mononuclear cells depleted of CD34+ cells (referred to herein as “MNCs”) were used for negative controls. In some experiments, CD34+ cells were isolated from other sources e.g., umbilical cord blood and from patients. These various CD34+ cells were used to evaluate the angiogenic potential and miRNA contents of the different exosome preparations.

Adult stem cell-derived exosomes have distinguishing features. For example, exosomes produced by this method are a generally homogenous population and are approximately 30-100 nm in diameter. The exosomes have a distinct cup-shaped morphology as visualized by electron microscopy.

In some embodiments, the exosomes have a characteristic density of 1.1 to 1.18 g/ml (alternatively, g/cm³ or g/cc) and contain the proteins TSG101 and CD63. In some embodiments, the exosomes contain CD34+ protein on their surface. The exosomes may have other angiogenic proteins on the surface or in the lumen. In addition, the exosomes may contain mRNAs and microRNAs in the lumen. In addition, CD34+ exosomes significantly increase the proliferation and induce tube formation of human umbilical-vein endothelial cells. The tube formation induced by CD34+ exosomes is dose dependent and similar to the effect of 100-fold greater amount of intact CD34+ cells. In vivo, neovascularization and incorporation of mouse endothelial (CD31) cells is significantly higher with CD34+ exosomes than with CD34+ cells. In some embodiments, the CD34+ exosomes are taken up by the cells in target tissues, where they may transfer mRNA, microRNA, or proteins to the host tissue or cells, thereby modifying the translation of proteins. In some embodiments, the CD34+ exosome secretion, surface marker proteins, and the level of angiogenic protein could depend on the disease conditions. One of skill in the art would understand that modifications of these exemplary embodiments could also result in suitable exosome preparations.

The present technology is not limited in the cells from which exosomes may be prepared. For example, sources of stem cells include, but are not limited to, cord blood, bone marrow, peripheral blood, brain, spinal cord, blood vessels, skeletal muscle, skin, teeth, heart, gut, liver, ovarian epithelium, amniotic fluid, umbilical cord, urine, and testis.

Moreover, exosomes may be prepared from a variety of cells depending on the therapy required. Exosomes are secreted by almost all cell types in an organism, including cell types of hematopoietic origin and cell types of nonhematopoietic origin. For example, exosomes are secreted from B cells, dendritic cells (Viaud et al, 2010, Cancer Res, 70(4): 1281), mast cells, T cells, platelets, intestinal epithelial cells, tumor cells, Schwann cells, neuronal cells, reticulocytes, and astrocytes (Chaput & Théry, Semin Immunopathol, 2011, 33(5): 419).

Further, synthetic vesicles that mimic the structure and/or properties of the cell-derived exosomes may be employed.

In addition to a common set of membrane and cytosolic molecules, exosomes harbor unique subsets of proteins, reflecting their cellular source (Raimondo et al, Proteomics, 2011, 11(4): 709). Because exosomes possess membrane and luminal components from their excreting cells, exosomes can perform functions related to the excreting cells from which they are derived. For example, certain cells of the immune system, such as dendritic cells and B cells, secrete exosomes that may play a functional role in mediating adaptive immune responses to pathogens and tumors (Aung et al, 2011, Proc Natl Acad Sci USA, 108(37): 15336; Bobrie et al, Traffic, 2011; Chaput & Théry, Semin Immunopathol, 2011, 33(5): 419). In addition, exosomes secreted by synaptic neurons may mediate neuronal plasticity, which may be important for memory and learning. Moreover, exosomes may carry protein, nucleic acids, and other cellular components in their lumen or membrane for delivery to secondary cells.

For example, both mRNA and microRNA have been found in exosomes and microvesicles excreted from particular types of cells (see, e.g., U.S. Pat. No. 8,021,847). This RNA can be transferred from the excreted exosome to another cell, most likely through fusion of the exosome to the recipient cell membrane. For example, mast cell-derived exosomes were found to contain a defined set of mRNAs and microRNAs that modulated transcription in recipient cells (Valadi, Nat Cell Biol, 2007, 6: 654). Similarly, embryonic stem cells secrete exosomes highly enriched in specific mRNAs, which can be transferred to and induce phenotypic changes in hematopoietic progenitor cells. Consequently, exosomes find use to deliver other oligonucleotides and therapeutically useful entities. For example, one can isolate exosomes from particular cell types that produce particularly desirable components useful for therapy and use those exosomes to deliver the therapeutic payload to a subject in need of therapy (Alvarez-Erviti et al, Nature Biotechnol, 2011, 29(4): 341). Cells may be engineered to express desired components that are taken into exosomes. Further, in some embodiments, desired agents are introduced into exosomes that have already been isolated from cells.

Autologous exosomes derived from a subject's cells are typically recognized as “self” by the subject's immune system. Consequently, exosomes isolated from a subject's cells can be loaded with exogenous payloads for administration to the subject with a minimal immune response. Such payloads include, for example, DNA, mRNA, microRNA, drugs, or other small molecules useful for therapy. Alternatively, allogeneic exosomes can be prepared from an immune compatible donor for administration to a subject. Furthermore, by incorporating the required self-recognition components into allogeneic exosomes, immune compatible exosomes can be prepared from cells isolated from any allogeneic source.

The cells used to prepare exosomes may be isolated from a living organism or from cells grown in culture. For example, the cells may be isolated from an animal, or more specifically from a mammal such as a human or a mouse.

Also, artificial vesicles (e.g., exosomes) can be assembled from synthetic liposomes or vesicles, the therapeutic payload to be delivered, and the particular components required by exosomes for effective delivery of their contents to recipient cells. Many types of amphipathic entities can form liposomes under thermodynamically favorable physical and chemical conditions. For example, liposomes can be produced using various cells, cell extracts, cell fractions, or other biological, chemically defined, or biologically-derived components as starting materials. In biological systems and under biologically relevant in vitro conditions, the amphipathic components are generally lipids, proteins, detergents, and mixtures thereof. Some particular types of biological amphipathic compounds include, but are not limited to, phospholipids, cholesterol, glycolipids, fatty acids, bile acids, and saponins. Liposomes can be prepared in vitro using a variety of techniques to obtain different lamellarity, size, trapped volume, and solute distribution. Some techniques used to produce vesicles include hydration, mechanical dispersion in water, freeze-thaw, reverse phase hydration from organic solvent, reverse phase evaporation, extrusion, sonication, detergent solubilization and removal, French press, dehydration-rehydration, and combinations thereof. Components that may be important for assembling synthetic exosomes are specific integrins, tetraspanins, MHC Class I and II antigens, CD antigens, and cell-adhesion molecules. In addition, cytoskeletal proteins, GTPases, clathrin, chaperones, and metabolic enzymes may be used. Finally, synthetic exosomes may also utilize mRNA splicing and translation factors, as well as several proteins such as HSP70, HSP90, and annexins.

### Therapies

As shown herein, exosomes produced from adult stem cells promote tissue regeneration and repair via angiogenesis in a similar manner as the stem cells from which the exosomes are derived. Accordingly, exosomes derived from adult stem cells (e.g., CD34+ stem cells) are useful as a replacement for stem cell therapy in tissue repair and regeneration. For example, exosomes are useful in therapies directed toward healing tissue damaged by ischemia. Additional indications are cardiovascular disease, myocardial or other infarction, chronic wounds, ulcer, clogged vessels, damaged vessels, stenotic vessels, atherosclerosis, angina, peripheral vascular disease, critical limb ischemia, ischemic heart disease, hypoxic tissues, heart failure, congestive heart failure, and bone marrow diseases. Moreover, indications include degenerative diseases such as Alzheimer's disease, diabetes, Parkinson's disease, and cancer. The therapy is also appropriate for subjects who require wound healing, scar reduction, or tissue regeneration. Additional indications are bone marrow transplant, tissue damage from stroke, hemorrhage, thrombosis, embolism, or hypoperfusion. Stem cell-derived exosomes are also useful in therapeutic angiogenesis and revascularization involving formation of endothelial cells. The angiogenic property can be mediated by the proteins and RNA present in the exosome lumen or on the exosome surface.

Not only are exosomes a useful tool for mediating changes in host cell expression through expression and delivery of molecules involved in angiogenesis promotion, but also stromal remodeling, chemoresistance, and genetic intercellular exchange. Moreover, entire signaling pathways may be delivered via growth factor and receptor transfer to recipient cells.

Therapies are not limited to the types of cells used to prepare exosomes. For example, dendritic cell-derived exosomes are immunogenic and can thus promote tumor rejection and eradication. Specifically, dendritic cell- and tumor cell-derived exosomes loaded with tumor antigen induce tumor antigen-specific CD8 cytotoxic T-lymphocyte responses and antitumor immunity in animals such as humans.

In addition, exosomes from a specific cell type carrying a specific protein or RNA associated with any disease or other medical condition can be used as a diagnostic tool. Specifically, exosomes provide protein and RNA biomarkers useful for detecting disease, monitoring disease evolution, and monitoring a subject's response to therapy. One example of a source of exosomes for evaluating biomarkers is urine. In addition, exosomes isolated from peripheral blood, plasma, and serum are useful for detecting and monitoring cancer, including tissue invasion and metastasis by cancer cells, in a subject (Skog et al, Nat Cell Biol, 2008, 10(12): 1470). Exosomes are also useful for diagnosing and monitoring the pathogenesis of various other diseases, such as atherosclerosis, thromboembolism, osteoarthritis, chronic renal disease, and pulmonary hypertension, gastric ulcers, bacterial infections, and periodontitis

It has been shown that exosomes can mediate antigen presentation in parallel with dendritic cells, B-cells, and macrophages (Testa et al, J Immunol, 2011 185(11): 6608, Bobrie et al, Traffic, 2011). Thus, in some embodiments, provided herein are cell-free, exosome-based compositions as therapy in malignant diseases via their ability to induce an immune response (e.g., use as vaccines).

The exosome compositions also find use in research settings. For example, exosomes can be used in drug screening to monitor the effects of a pharmaceutical preparation. In addition, exosomes provide important tools for studying models of disease in a research setting. Exosomes prepared from cells of a disease model system are useful for monitoring disease progression and the disease's response to therapy.

### EXAMPLES

The following examples are provided to demonstrate and further illustrate certain preferred embodiments and aspects of the present technology, and they are not to be construed as limiting the scope of the technology.

### Methods

All experimental protocols were approved by the Northwestern University Animal Care and Use Committee. CD34+ cells and CD34+ cell-depleted mononuclear cells (MNCs) were cultured using standard methods. Electron microscopy, dynamic light scattering (DLS), flow cytometry, and immunoblotting analyses were performed according to established protocols. The angiogenic activity of cultured human umbilical-vein endothelial cells (HUVECs) was evaluated by the Matrigel tube-formation assay, proliferation was evaluated by 5-bromo-2-deoxyuridine incorporation, and viability was assessed by the MTS assay. In vivo angiogenesis was evaluated in nude (nu/J) mice using the Matrigel plug and corneal angiogenesis assays. Quantified results are presented as mean±the standard deviation; comparisons between groups were evaluated with the Student t test; P<0.05 was considered significant.

Cells and Culture

CD34+ cells and the CD34+-cell-depleted mononuclear cells (MNCs) were purified from mobilized peripheral-blood mononuclear cells (AllCells LLC, Emeryville, Calif.) with an Isolex 300i device (Baxter Healthcare); cell purity was 85-95% as determined by flow cytometry. Both CD34+ cells and MNCs (250,000 cells/ml) were cultured in X-VIVO 10 serum-free cell-culture medium (Lonza Group Ltd, Basel, Switzerland) containing 0.25% human serum albumin and supplemented with 100 ng/ml Flt-3L, 100 ng/ml stem-cell factor, and 20 ng/ml vascular endothelial-growth factor. Human umbilical-vein endothelial cells (HUVECs) (Cambrex Corporation, East Rutherford, N.J.,) were maintained in endothelial growth medium-2 (EGM™-2; Cambrex Corporation) and starved in EBM-2 medium containing 0.25% fetal bovine serum for 24 hours before cell assays were performed.

Exosome Purification

Cells were cultured for 40 hours and exosomes were collected and ultrapurified as described previously (see, e.g., Théry, C. et al. “Isolation and characterization of exosomes from cell culture supernatants and biological fluids” in Curr Protoc Cell Biol. 2006, Chapter 3: Unit 3.22, which is expressly incorporated herein by reference in its entirety for all purposes). Briefly, the cells and conditioned media were separated by centrifugation (800×g for 5 minutes); the conditioned media was clarified by centrifugation (14,000×g for 20 minutes) and the exosomes were collected by ultracentrifugation (100,000×g for 1 hour) on a 30% sucrose-D₂O solution (density ˜1.127 g/cm³), then washed in PBS and pelleted. The purified exosome fraction was re-suspended in PBS for use.

Electron Microscopy

Cells were fixed with 4% paraformaldehyde and 1% glutaraldehyde in 0.1 M sodium cacodylate buffer (pH 7.2) (Electron Microscopy Sciences, Hatfield, Pa.) for 3 hours at room temperature, washed with cacodylate buffer, postfixed in 1% osmium tetroxide, progressively dehydrated in a graded ethanol series (50-100%), and embedded in Epon. Thin (1-mm) and ultrathin (70- to 80-nm) sections were cut from the polymer with a Reichert (Depew, N.Y.) Ultracut S microtome, placed on copper grids, and briefly stained with uranyl acetate and lead citrate. Exosomes were fixed with 2% paraformaldehyde, loaded on 300-mesh formvar/carbon-coated electron microscopy grids (Electron Microscopy Sciences, PA), post-fixed in 1% glutaraldehyde, and then contrasted and embedded as described previously (see, e.g., Théry, C. et al. “Isolation and characterization of exosomes from cell culture supernatants and biological fluids” in Curr Protoc Cell Biol. 2006, Chapter 3: Unit 3.22). Transmission electron microscopy images were obtained with an FEI (Hillsboro, Oreg., USA) Tecnai Spirit G2 transmission electron microscope operating at 120 kV.

Dynamic Light Scattering

Exosomes were suspended in phosphate-buffered saline (PBS) containing 2 mM ethylenediaminetetraacetic acid (EDTA); then, dynamic light-scattering measurements were performed with a Zetasizer Nano ZS (Malvern Instruments Ltd, Worcestershire, UK). Intensity, volume, and distribution data for each sample were collected on a continuous basis for 4 minutes in sets of three. At least three different measurements from three different samples were performed for each exosome population.

Flow Cytometry

Flow cytometry analysis was performed as described previously (see, e.g., Théry, C. et al. “Isolation and characterization of exosomes from cell culture supernatants and biological fluids” in Curr Protoc Cell Biol. 2006, Chapter 3: Unit 3.22). Exosomes were conjugated to 4-μm latex beads for analysis because their diameter (<0.1 nm) is smaller than the detection limit (˜0.1-0.2 nm) of the flow cytometer. Briefly, exosomes from 5×10⁶ cells were incubated overnight at 4° C. with 2.5×10⁵ aldehyde/sulfate latex beads (Invitrogen, Carlsbad, Calif.) and then blocked with 100 mM glycine for 30 minutes at room temperature to saturate any free binding sites that remained on the beads. To detect the presence of CD63 and CD34, the exosome-coated beads were resuspended in 500 μl PBS containing 0.5% human serum albumin (HSA) and 2 mM EDTA; then, 100 μl of the beads were incubated with fluorescein-isothiocyanate (FITC)-conjugated anti-CD63 or FITC-conjugated anti-CD34 antibodies (Beckman Coulter, Inc., Brea, Calif.) for 30 minutes at 4° C. For phosphatidylserine detection, the beads were resuspended in 100 μl of Annexin-V-FLUOS labeling solution (Annexin-V-FLUOS Staining Kit, F. Hoffmann-La Roche Ltd, Basel, Switerland) and incubated for 10 minutes at 25° C. Non-specific binding/labeling was inhibited by the addition of FcR blocking reagent (Miltenyi Biotec Inc., Auburn, Calif.); the threshold for negative staining was obtained by incubating exosome-free, glycine-blocked beads with each antibody, and additional experiments were performed with identical concentrations of control IgG antibodies to correct for non-specific binding.

For direct detection of exosomes by the flow cytometer, exosomes from either CD34+ cells or MNCs were first labeled with FITC-conjugated anti-CD34 antibodies (Beckman Coulter, Inc., Brea, Calif.) or an isotype control, then labeled with cellvue maroon dye (Polysciences, Inc, PA) for detection by the flow cytometer. Flow cytometry data were acquired on a BD LSRII (BD Franklin Lakes, N.J.) flow cytometer and analyzed with FlowJo software (Tree Star, Ashland, Oreg.).

Transfection of Cy3-labeled RNA into cells was performed with the lipofectamine reverse-transcription method.

In-Vitro Matrigel Tube Formation Assay

HUVECs (2.5×10⁴, serum-starved overnight) were incubated with PBS, 2.0×10⁴ CD34+ cells, 2.0×10⁴ CD34+ MNCs, or with the conditioned media, exosomes, or exosome-depleted conditioned media from 2.0×10⁴ CD34+ cells or MNCs into 48-well plates that had been coated with 150 μL of growth-factor-reduced Matrigel™ (BD). Tube formation was examined by phase-contrast microscopy 6-8 hours or 24 hours later. Each condition in each experiment was assessed in duplicates and tube length was measured as the mean summed length of capillary-like structures in 2 wells by examining high-power fields (HPFs, 2.5×) in each well. Multiple (e.g., 3-4, 6-9, etc.) experiments were performed for each condition. Tube length is expressed as a percentage of the length for PBS-treated HUVECs.

Dose-response experiments were performed by incubating HUVECs with exosomes from 1.5×10⁵ CD34+ cells and serially diluted to 1/3, 1/9, 1/27, 1/100, 1/300, and 1/900 of the initial concentration (initial concentration=1).

In Vitro Proliferation and Viability Assays

Cell proliferation was evaluated via 5-bromo-2-deoxyuridine (BrdU) incorporation. Serum-starved HUVECs (1×10⁴) were incubated with 10 μM BrdU and 2.0×10⁴ CD34+ cells, 2.0×10⁴ MNCs, or with exosomes from 2.0×10⁴ CD34+ cells or MNCs for 24 hours, and then washed and fixed with 4% paraformaldehyde at 4° C. Ten minutes later, the HUVECs were washed in PBS with 1% Triton X-100 for 5 minutes, incubated on ice in 1 N HCl for 10 minutes, incubated at room temperature in 2 N HCl for 10 minutes, and incubated at 37° C. for 20 minutes. The HCl was neutralized via three 5-minute washes with borate buffer (0.1 M), and then the HUVECs were washed in PBS with 1% Triton X-100 at room temperature for 3 minutes, blocked with 5% normal goat serum and 1% Triton X-100 in PBS for 1 hour, and incubated overnight with immunofluorescent sheep anti-BrdU antibodies (Abeam Inc., Cambridge, Mass., USA); nuclei were counterstained with DAPI. Cells were viewed at 10× magnification and BrdU+ cells were counted in 10 HPFs per well, 2 wells per condition.

Cell viability was evaluated via the MTS assay. HUVECs (1×10⁴ cells/well) were seeded on 96-well flat-bottomed plates and incubated with 2.0×10⁴ CD34+ cells or MNCs, or with exosomes from 2.0×10⁴ CD34+ cells or MNCs, for 20 hours at 37° C.; then, the MTS assay reagent (Promega Corporation, Madison, Wis.) was added to the wells and HUVECs were incubated for 3 hours at 37° C. Viability was evaluated by measuring absorbance at 490 nm with a 96-well ELISA plate reader (SpectraMaxPlus, Molecular Devices, Sunnyvale, Calif.) in at least 6 wells per experiment and 3-7 experiments per condition.

Western Blotting

Cells or purified exosomes were lysed with 0.1 M Tris, 0.3 M NaCl, 0.1% SDS, 0.5% sodium deoxycholate, and 1% Triton X-100 in a cocktail of antiproteases (Sigma-Aldrich Corporation, St. Louis, Mo.); then, the nuclei and membranes were cleared by centrifugation (15,000×g for 10 minutes). Protein extracts were separated on an 8% SDS-PAGE gel, blotted on Immobilon (Millipore, Billerica, Mass.) with TSG101 (4A10; Abcam Inc.), and visualized with enhanced chemoluminescence substrate (Thermo Fisher Scientific, Rockford, Ill.). Images were acquired with a Chemidoc XRS (Kodak, Rochester, N.Y.).

In-Vivo Matrigel-Plug Assay

Ice-cold Matrigel (0.5 ml/plug; BD) was mixed with heparin (1 mg/ml) and PBS, 5.0×10⁵ CD34+ cells or exosomes from 5.0×10⁵ CD34+ cells and then subcutaneously injected into the flanks of 6- to 8-week-old male nude mice (Nu/J; The Jackson Labortory, Bar Harbor, Me.). Mice were anesthetized with inhaled isoflurane (2-4%) before injection. 7-14 days later, the plug was excised and washed with PBS. To visualize vessel-like endothelial structures, the plug was fixed in methanol and sectioned; then, endothelial cells were stained with biotinylated isolectin B4 (Vector Laboratories Inc, Burlingame, Calif.), and nuclei were stained with hematoxylin. Images were acquired with an Olympus Vanox bright microscope. For flow-cytometry analysis of endothelial-cell migration, the plug was digested with 0.1% collagenase/dispase (F. Hoffmann-La Roche), 10 mm MgCl₂, and 200 units/ml DNase I (F. Hoffmann-La Roche) in 10% fetal calf serum/PBS for 1 hour at 37° C. After digestion, cells were dispersed 4-5 times with a 21 gauge needle, passed through a 70-mm filter (BD), and stained with phycoerythrin-conjugated rat anti-mouse-CD31 antibodies (BD). Control assessments were performed with phycoerythrin-conjugated rat immunoglobulin G2a isotype (Invitrogen). Flow cytometry data were acquired on a FACScan (BD) flow cytometer and analyzed with FlowJo software (Tree Star).

Mouse Corneal Angiogenesis Assay

Pellets were prepared and implanted in the corneas of 6- to 8-week-old male nude mice (Nu/J; The Jackson Laboratory) as described previously (see, e.g., Rogers M. S. et al. Nat Protoc. 2007, 2 :2545-50, incorporated herein in its entirety for all purposes). Briefly, 5 mg sucrose octasulfate-aluminum complex (Sigma-Aldrich Corporation) and 10 μL of 12% hydron in ethanol were mixed and partially dried; then, exosomes from 5.0×10⁵ CD34+ cells or MNCs were added, the mixture was pelleted on a 400-μm nylon mesh (Sefar America Inc., Depew, N.Y.), and the pellets were dried for 5-10 minutes. Pellets were implanted in the corneas of mice that had been anesthetized via intraperitoneal injection of 125 mg/kg Avertin. One week after implantation, the mice were intravenously injected with 50 μl of fluorescein-conjugated BS1-Lectin I (Vector Laboratories) and sacrificed 15 minutes later. Eyes were harvested and fixed with 1% paraformaldehyde; then, the corneas were excised and mounted. Angiogenesis was evaluated via BS1-Lectin I fluorescence and quantified with ImageJ software.

Mouse Hind Limb Ischemia Model

BalbC nude mice (8-10 weeks old) were anesthetized with Isoflurane delivered at approximately 2%. All animals were placed on a warm circulating water pad to maintain body temperature throughout the procedure. Prior to the ischemic procedure and immediately following it, measurements of blood flow in both thighs were taken as a baseline and to confirm ischemia. The left thigh region was surgically prepped with betadine followed by alcohol. The depth of anesthetic plane was assessed by lack of toe pinch reflex and a 5-mm incision was made on the left thigh region. A ligation was made around the femoral artery and all arterial branches were removed. A small segment of the artery was then dissected free. Mice were randomly assigned to receive the treatments of PBS, CD34+ cells, CD34+ cell conditioned media, CD34+ Exosomes, CD34+ exosome-depleted conditioned media, or MNC exosomes immediately after creating hindlimb ischemia. The treatments were applied directly into the ischemic hindlimb in a 20-0 volume and injected at 4 different locations. The connective tissues of the sub cutis were closed with interrupted 6-0 polypropylene suture and the skin closed with wound clips or 6-0 polypropylene suture. Prior to recovery from anesthesia, each animal was administered Buprenex (0.2 mg/kg IP) and meloxicam solution (0.001 mg/g) was administered in the water for up to ten days post operatively to minimize any pain as a result of surgery.

For laser Doppler measurements of the ischemic and control limbs, animals were anesthetized with Isoflurane (2%) and LDPI measurements were taken at 7, 14, 21, and 28 days following hind limb ischemic surgery. Ischemic and non-ischemic tissues were harvested at day 28 for histological analyses. Before sacrifice, the mice were injected with 50 μg of BS-1 lectin to identify the mouse vasculature.

For limb functional assays, limb motor function was scored as follows—1: no limb use; 2: no foot use, limb use only; 3: restricted foot use; 4: no active toe use (spreading), foot use only; and 5: unrestricted limb use. Limb salvage (i.e. no tissue necrosis) was scored as follows—1: limb amputation; 2: foot amputation; 3: toe(s) amputation; 4: necrosis, nail loss only; 5: full recovery. n=7-12 per group.

Capillary density was determined by imaging lectin-stained capillaries in the ischemic limb of mice treated with PBS, CD34+ cells, CD34+ CM, CD34+ Exo, CD34+ Exo-depleted CM, or MNC Exo (all derived from equal number of cells). At least 10 high-power field images per condition (either ischemic or non-ischemic) from at least 4 mice per group were counted and averaged. Values are reported as the ratio of capillary density in the ischemic to non-ischemic limb. *P<0.05.

MicroRNA Quantification

Total RNA from the CD34+ cells, CD34+-depleted MNCs, and their respective exosome preparations were extracted using the miRNeasy Mini Kit (Qiagen) according to the manufacturer's protocol (including a DNase step). RNA concentrations were verified on a NanoDrop Spectrophotometer (NanoDrop) and the quality of total RNA was assessed using Agilent 2100 Bioanalyzer Pico Chips (Agilent). Equal amounts of RNA (5 ng) were reverse transcribed using the Taqman MicroRNA Reverse Transcription Kit (Applied Biosystems) using a specific miRNA primer to generate cDNA for use with individual Taqman MicroRNA Assays (Applied Biosystems). Real-time Reactions were performed in triplicate on a 7500FAST Real-Time PCR system (Applied Biosystems). Cₜ values were averaged and normalized to the U6 RNA (e.g., RNU6B). Experiments were performed with an n=2-6. Relative expression was determined by the ddCₜ comparative threshold method.

MicroRNA Microarray

miRNA profiling was performed using Affymetrix miRNA microarrays.

Cy3 miRNA Uptake

Cy3 miRNA (30 pmol) was transfected into CD34+ cells (125,000 cells/500 μl media) using lipofectamine-reverse transcription. Untreated cells, lipofectamine alone, and Cy3-treated cells were used for controls. After 24 hours, the cells were washed and re-plated. Exosomes were isolated after ˜40 hours and then incubated with HUVECs (either GFP positive or regular HUVECs for live imaging). The CD34+ cells and a portion of exosomes tagged with 4-μm beads were used for flow cytometry analysis to verify Cy3 transfection. The Cy3 or control exosome-treated HUVECs were imaged in a Nikon C1S1 confocal microscope.

### Example 1

### Electron Microscopy and Physical Characterization of Exosomes

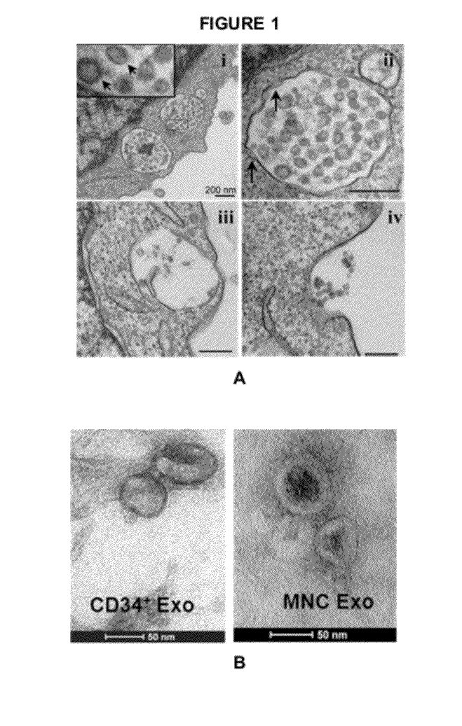

Experiments performed during the development of embodiments of the technology provided herein demonstrated the presence of multivesicular bodies (MVB) in the cytoplasm of CD34+ cells. In electron micrographs, MVB were identified that harbored numerous bilipidic membrane-bound exosome-like vesicles of approximately 50 nm in diameter (e.g., approximately 30 nm-100 nm or 40 nm-90 nm in diameter). Some micrographs showed instances of the MVB membrane invaginating inward to initiate the biogenesis of exosomes and some micrographs showed instances of the MVBs fusing to the plasma membrane and releasing the exosome-like vesicles into the media (FIG. 1a, (i)-(iv)).

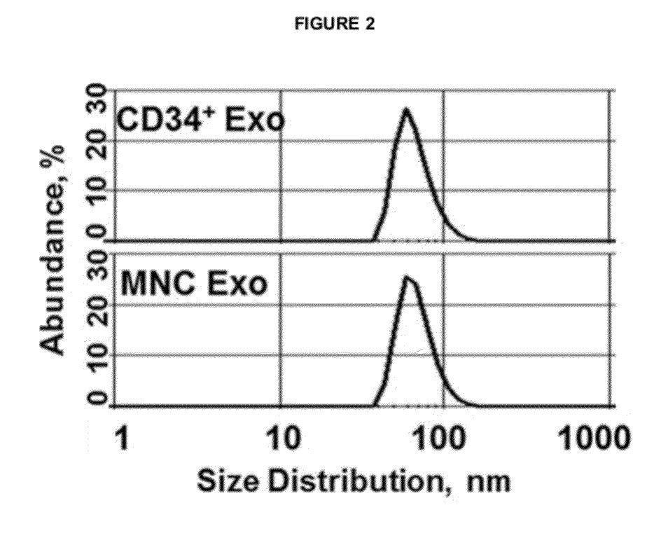

In addition, physical characteristics of prepared vesicles were monitored during the development of embodiments of the technology. Exosomes were isolated from the conditioned media (CM) in which either CD34+ cells or MNCs were cultured. After the exosomes were isolated, they were prepared for electron microscopy. The electron micrographs (FIG. 1b) showed that the exosomes in the preparations had a similar size (e.g., approximately 40-90 nm or 30-100 nm in diameter) and cup-shaped morphology as has been reported previously. Sucrose density gradient analysis showed that the exosome preparations had a flotation density (1.127 g/cm³, floated on 30% sucrose-D₂O solution) that was similar to that previously reported. Dynamic light scattering (DLS) analysis was used to assess the purity and to determine the mean hydrodynamic radius of the exosomes in each preparation. The analysis shows that the mean hydrodynamic radius for the (CD34+ exosomes is 50±7.8 nm and for the MNC exosomes is 75±0.4 nm (FIG. 2). The single peak in the DLS data indicates that the exosome preparations are free of contamination. Also, a preparation of exosomes that was thawed after approximately 6 months of storage in a frozen state exhibited the same size as freshly prepared exosomes. This result indicates that storing the exosomes in a frozen state (e.g., at −80° C.) does not compromise their physical morphology.

### Example 2

### Exosome Marker Proteins

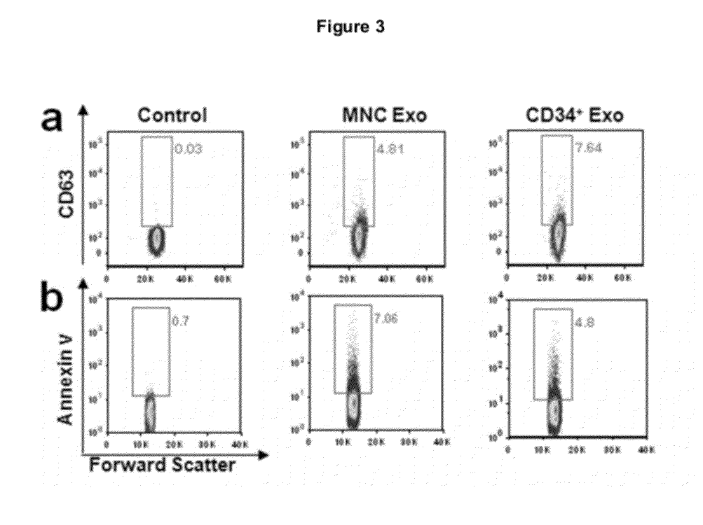

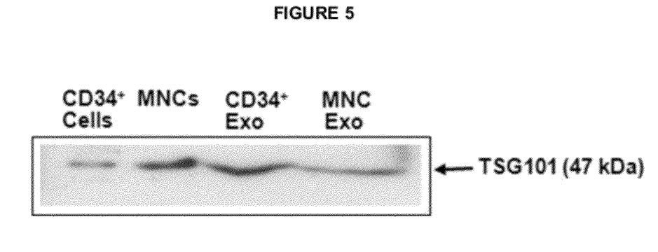

During the development of embodiments of the technology provided herein, flow cytometry experiments were conducted that demonstrated that the membranes of exosomes from both CD34+ cells and MNCs contained the exosome surface marker protein CD63 (FIG. 3a) and the lipid phosphatidylserine. The presence of phosphatidylserine was demonstrated by its binding to annexin V (FIG. 3b). Exosomes were tagged with 4-μm Latex beads to increase their size for detection by the flow cytometer. In addition, exosomes from both CD34+ cells and MNCs contained the exosomal luminal marker protein TSG101 (FIG. 5).

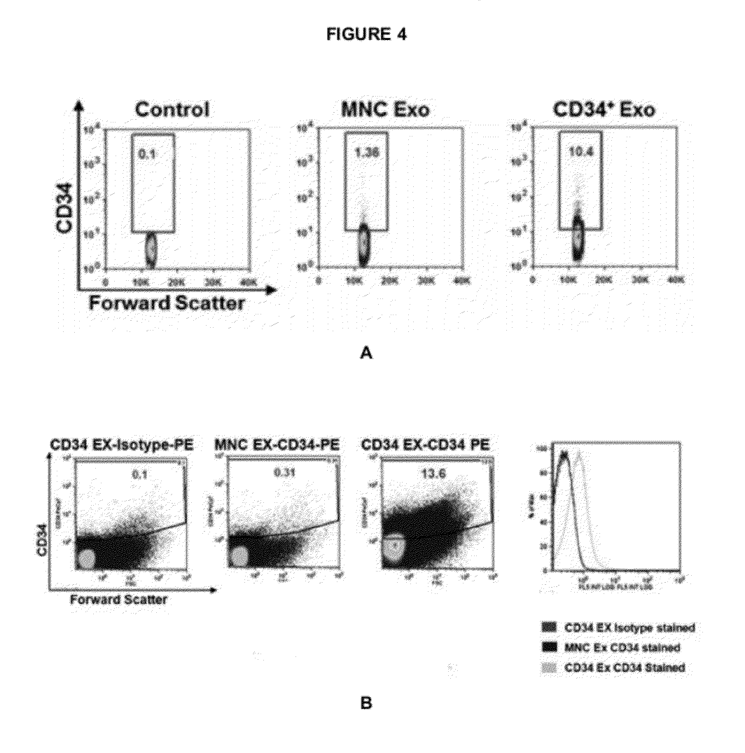

Further, CD34 protein was present on the surface of exosomes from CD34+ cells but not on exosomes from MNCs (FIG. 4). Exosomes labeled with fluorescence for detection by flow cytometry demonstrated a spectral shift indicating the presence of CD34 protein on the surface of the CD34+ cell-derived exosomes (FIG. 4b, histogram). These data are consistent with previous reports that exosomes carry marker proteins that are specific for the secreting cell. Collectively, these observations confirm that both CD34+ cells and MNCs secrete exosomes and that the exosomes secreted by each cell population are biochemically distinct (e.g., exosomes from CD34+ cells have CD34 protein but exosomes from MNCs do not).

### Example 3

### Angiogenic Activity of CD34+ Exosomes

3.1. CD34+ Exosomes Induce Angiogenesis of Endothelial Cells In Vitro

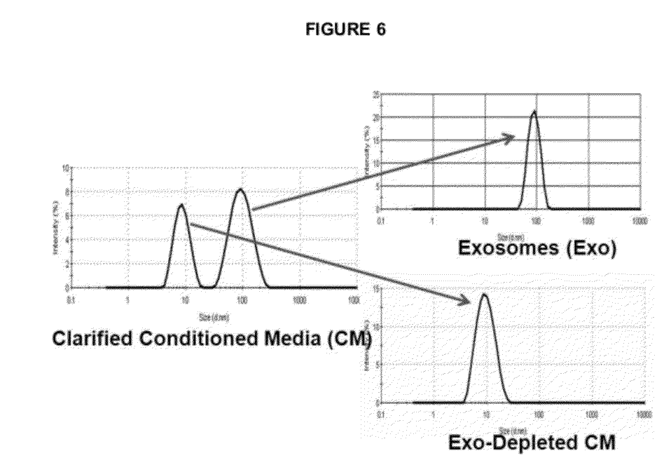

During the development of embodiments of the technology described, preparations comprising CD34+ cells, CD34+ cell secreted conditioned media (CM), CD34+ exosomes (Exo), and CD34+ Exo-depleted CM (representing the free floating proteins secreted by the cells) were evaluated as potential mediators of CD34+ cell induced neovascularization. In these experiments, the preparations were derived from similar numbers of cells. DLS analysis demonstrated the successful separation of exosomes (˜50 nm) from the exosome-depleted conditioned media containing proteins or protein aggregates of smaller size (˜10 nm) (FIG. 6).

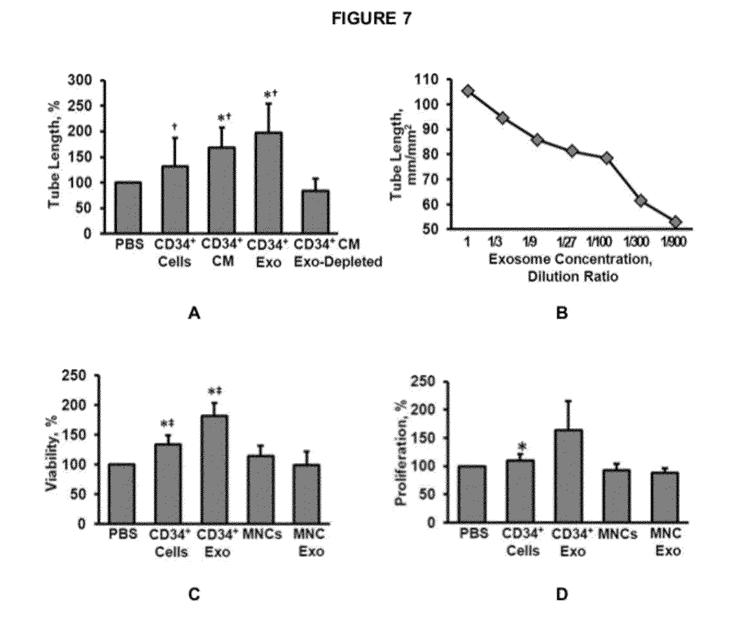

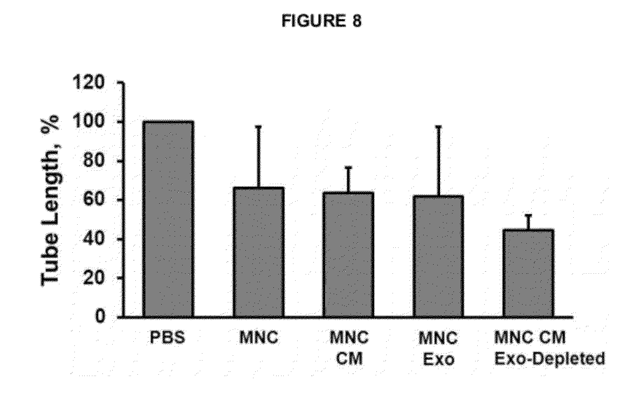

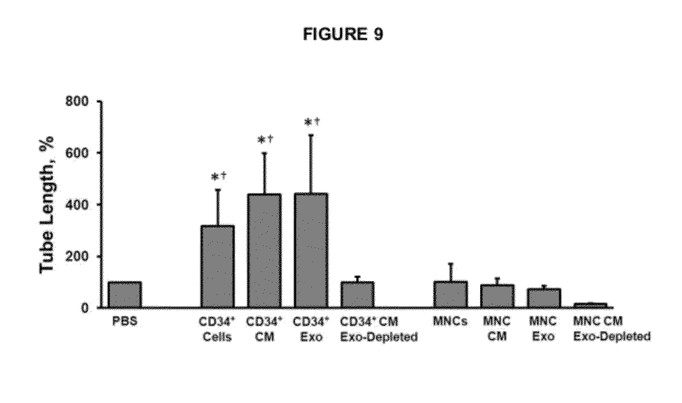

The in vitro angiogenic activities of the CD34+ cell preparations were evaluated by the in vitro Matrigel tube formation assay and compared to the non-therapeutic MNCs and MNC-derived CM, MNC Exo, and MNC Exo-depleted CM. In the assay, 2.5×10⁴ human umbilical vein endothelial cells (HUVECs) were cultured with phosphate-buffered saline (PBS), 2.0×10⁴ CD34+ cells, or with CM, exosomes, or exosome-depleted CM from 2.0×10⁴ CD34+ cells and plated on Matrigel (FIGS. 7a & 8). Tube length was significantly greater in HUVECs incubated with the CD34+ cell CM or with CD34+ exosomes than in HUVECs incubated with PBS; tube length for HUVECs incubated with the exosome-depleted CM was the same as for HUVECs incubated with PBS (FIG. 7a). These results suggest that CD34+ exosomes mediate the in vitro angiogenic activity seen for the CD34+ cell CM. Interestingly, CD34+ exosomes, similar to CD34+ cells, induced longer-lasting tubes in HUVECs measured at 24 hours of the assay (FIG. 9). Tube formation was less pronounced at lower exosome concentrations (FIG. 7b). HUVECs incubated with MNCs or MNC components (e.g., CM, exosomes, or Exo-depleted CM) did not differ significantly from PBS-treated HUVECs in inducing tube formation on Matrigel (FIG. 8).

The cell-culture medium comprised supplemental growth factors and may have contained soluble proteins secreted from the cells. While these components could have contributed to the angiogenic effects associated with CD34+ exosomes, the MNC exosomes were derived from MNCs cultured with the same growth factors; and thus the exosome-depleted conditioned media would have contained the same supplemental growth factors and any secreted soluble proteins. Since none of the MNC treatments stimulated angiogenic activity, the data indicate that the CD34+ exosome induced vessel growth.

3.2. CD34+ Exosomes Induce Cytoprotection and Proliferation of Endothelial Cells

During the development of some embodiments of the technology provided, experiments demonstrated that both CD34+ cells and CD34+ exosomes from the same number of cells significantly enhanced HUVEC viability (FIG. 7c) and proliferation (FIG. 7d) compared to the MNCs or MNC exosomes. HUVECs (1×10⁴) were incubated with PBS, 2.5×10³ cells, or exosomes from 2.5×10³ cells, and measured 20 hours later. Values are expressed as a percentage of the PBS-treated HUVECs. These data show that most of the in vitro angiogenic activity associated with CD34+ cells is mediated by exosomes. HUVECs incubated with MNCs or MNC exosomes did not differ significantly from PBS-treated cells in any functional parameter (FIGS. 7c, 7d, and 9).

3.3. CD34+ Cells from Cord Blood are Angiogenic

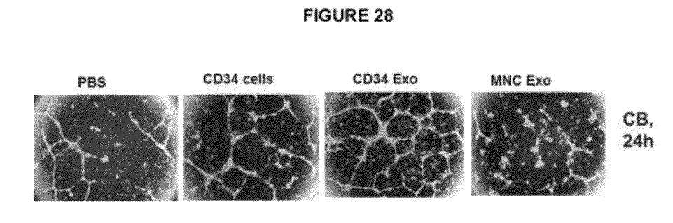

Consistent with the data above for the PB-derived CD34+ cells, EM data collected during the development of the present technology demonstrated that both the CD34+ cells and CD34+ exosomes isolated from umbilical cord blood (FIG. 28), but not the MNCs and MNC exosomes isolated from umbilical cord blood, were angiogenic.

3.4. CD34+ Exosomes Induce Angiogenesis In Vivo

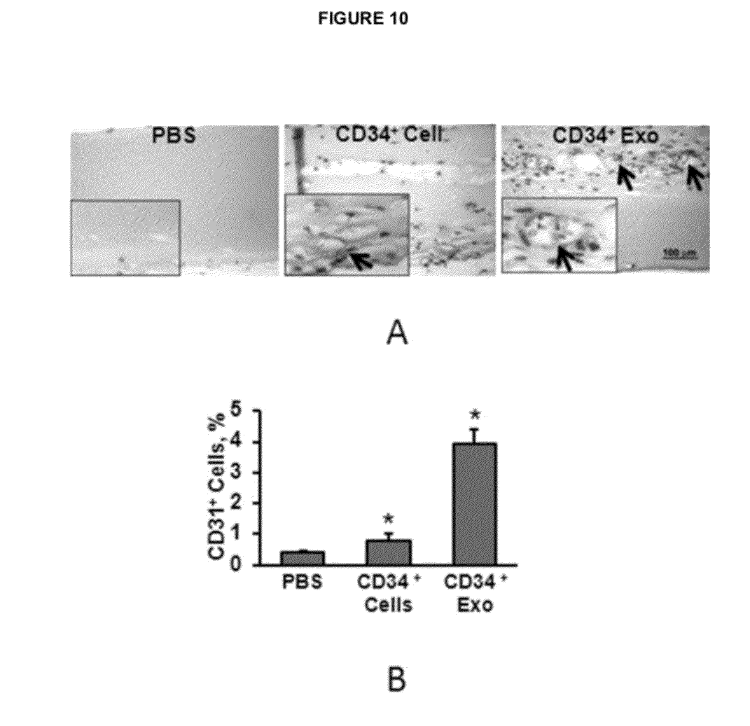

Experiments were performed to evaluate the angiogenic potency of CD34+ exosomes in vivo by performing Matrigel plug assays. The data collected indicate that both CD34+ cells and CD34+ exosomes from equal number of cells induced the formation of vessel-like endothelial structures (FIG. 10a) and significantly increased the proportion of endothelial cells in the Matrigel plug (FIG. 10b).

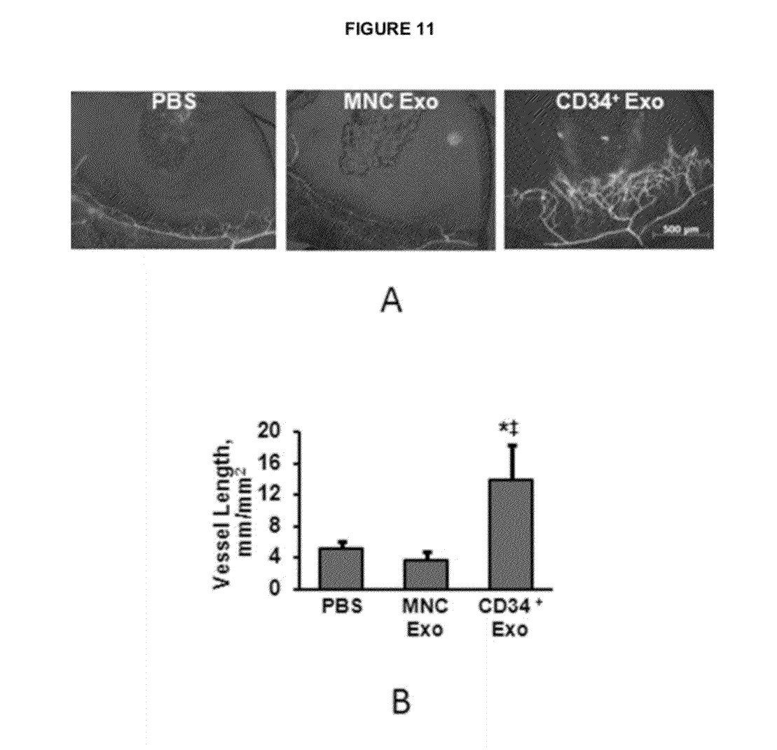

Additional experiments conducted during development of embodiments of the present technology demonstrated that exosomes induce angiogenesis in vivo. Pellets of hydron and sucralfate were prepared for implantation into mouse corneas. In separate experiments, the pellets included either exosomes from CD34+ cells or exosomes from CD34+-depleted MNCs. Pellets with either nothing added or containing FGF-2 were used as negative and positive controls, respectively. After implantation, angiogenesis was measured at day 7 by staining with fluorescent isolectin and assessing fluorescence under a microscope. Both FGF-2 and CD34+-derived exosomes induced angiogenesis as indicated by isolectin fluorescence. In the corneal angiogenesis assay, pellets containing CD34+ exosomes demonstrated significantly greater vessel growth compared to pellets containing MNC exosomes (FIG. 11). No angiogenesis was detected in mouse cornea treated with the negative control or with CD34+-depleted MNC-derived exosomes. The effect of CD34+ cells on corneal angiogenesis could not be evaluated, because the pellets could not be prepared with viable cells.

### Example 4

### Therapeutic Activity of CD34+ Exosomes

4.1. Functional Recovery with CD34+ Exosomes

During the development of embodiments of the technology provided herein, the murine hind-limb ischemia model was used to evaluate the potential of CD34+ exosomes as a therapy for ischemic diseases. PBS, CD34+ cells, CD34+ CM, CD34+Exo, CD34+ Exo-depleted CM, or MNC exosomes (as an experimental control) were administered by an intramuscular injection after the induction of critical ischemia by ligation and excision of the left femoral artery and all superficial and deep branches. To assess functional recovery after critical hind-limb ischemia, animals were assessed for tissue perfusion, limb salvage, and limb motor functions.

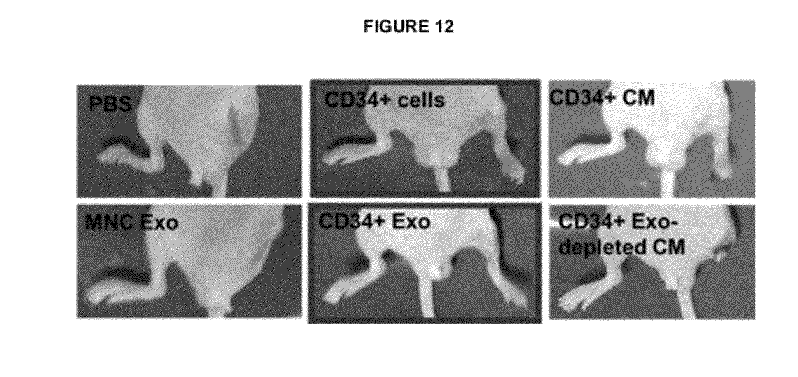

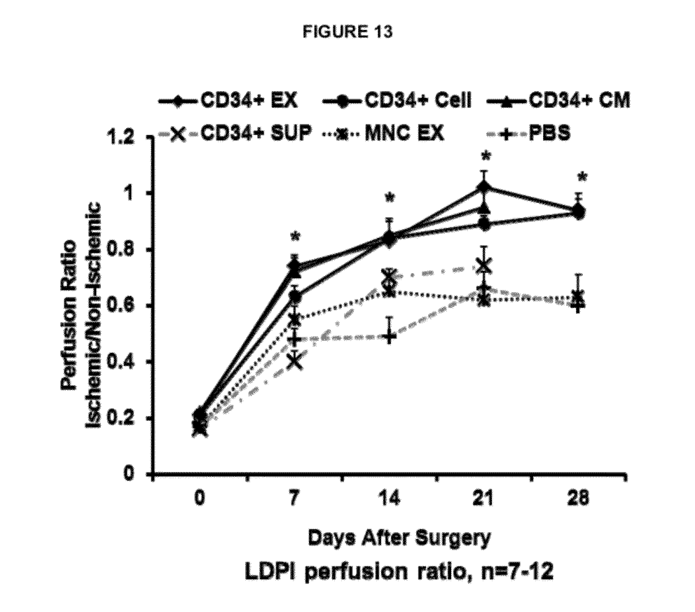

Tissue perfusion ratio. Physical examination of the ischemic leg after 7, 14, 21, and 28 days of surgery indicates rescue of the ischemic hind limb from limb amputation and tissue necrosis by treatment with CD34+ cells (FIG. 12). Identical effects were seen for treatment with CD34+ CM and CD34+ exosomes. Tissue perfusion was assessed by laser Doppler perfusion imaging (LDPI) in the ischemic hind limb and expressed as relative to the perfusion in the non-ischemic limb. The result of treatment with CD34+ exosomes was similar to CD34+ cells; both treatments produced significant improvements in tissue perfusion ratio at day 7 and continued to have a significantly higher perfusion ratio compared to treatment with PBS (limb perfusion ratios at day 28 were 0.94±0.2 (CD34+ exosomes), 0.93±0.17 (CD34+ cells), and 0.6±0.08 (PBS), with a P<0.05) (FIG. 13).

Parallel to these angiogenic results, the perfusion in the hind limb of animals treated with CD34+ CM containing exosomes was similar to the perfusion in the hind limb of animals treated with CD34+ Exo. However, depletion of exosomes from the CM (CD34+ Exo-depleted CM) resulted in loss of improved perfusion. This shows that CD34+ exosomes in the CM improve ischemic tissue perfusion. Animals treated with MNC exosomes isolated from an equal number of MNCs did not differ significantly compared to the PBS-treated control group (FIG. 13).

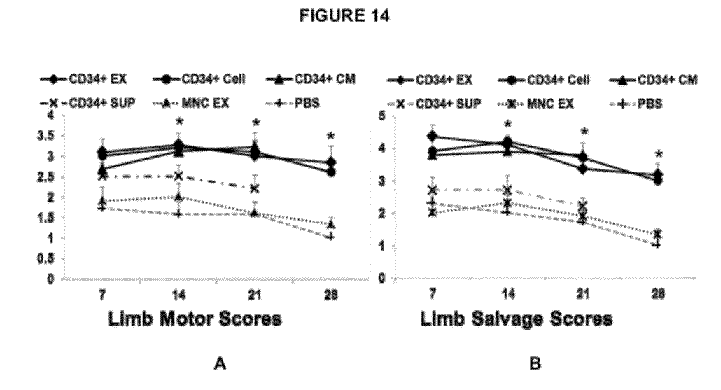

Limb salvage and limb motor ability. During the development of embodiments of the technology described herein, experiments were performed to assess treatment of the ischemic limb by exosomes. Limb salvage and limb motor functions were studied via established semi-quantitative scoring methods to evaluate tissue necrosis and amputation of ischemic limb (see Methods). The data showed a significant improvement in limb salvage score (3.2±1.1 versus 1.1±0.8; P<0.05, n=7-12) and motor score (2.83±1.3 versus 1.0±0.0; P<0.05, n=7-12) for the treatments with CD34+ exosomes as compared to treatment with PBS (FIG. 14). The beneficial effects of CD34+ Exo were similar to the beneficial effects of CD34+ cells and CM containing Exo (FIG. 14). These data suggest that the CD34+ Exo in the CM provide the key paracrine component promoting tissue repair.

4.2. Therapeutic Angiogenesis with CD34+ Exosomes

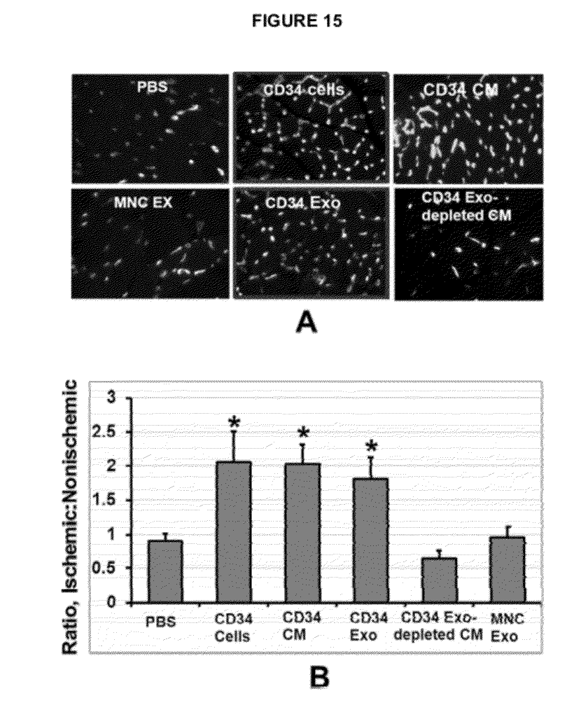

Experiments were performed during the development of embodiments of the technology provided herein to evaluate the beneficial effects of CD34+ exosome treatment on recovery of blood flow, motor function, and tissue salvage. The data demonstrated that beneficial effects of the CD34+ exosomes were associated with an effect on the microcirculation of the ischemic limb muscle. In particular, the number of lectin positive capillaries was quantified by immunofluorescence in the ischemic limb harvested at day 28 (FIG. 15a). The number of capillaries in the ischemic limb was expressed relative to the non-ischemic limb. There was a significant increase in the ratio of lectin-positive capillaries in the ischemic limb to lectin-positive capillaries in the non-ischemic limb in the animals treated with CD34+ exosomes as compared to the PBS-treated animals (FIG. 15b). The CD34+ exosomes produced effects similar to the CD34+ cell and CD34+ CM treatment groups. CD34+ exosome-depleted CM and MNC exosomes treatment had no significant effect on the capillary density. This pro-angiogenic effect of CD34+ exosomes on capillary microcirculation is consistent with the angiogenic activity of CD34+ exosomes in the in vivo Matrigel plug assay and corneal angiogenic assay.

In summary, these data demonstrated that adult human CD34+ stem cells secrete exosomes and that these exosomes induce angiogenic activity in isolated endothelial cells and in murine models of vessel growth. The improvements in tissue perfusion, limb salvage, motor function, and capillarization demonstrated the therapeutic utility of CD34+ exosomes for ischemic tissue repair.

### Example 5

### Molecular Composition of CD34+ Exosomes

In experiments performed during the development of embodiments of the technology provided herein, the protein and miRNA content of CD34+ exosomes and MNC exosomes were characterized and compared. It is contemplated that exosomes mediate intercellular communication by stimulating both receptor-mediated and genetic mechanisms through the transfer of functional proteins, RNA, or microRNA directly into the cytoplasm of target cells. Without being bound by any particular theory, the repertoire of specific molecules transported by CD34+ exosomes is likely to be more stable than molecules secreted directly into the extracellular matrix because the exosomal membrane protects the exosome contents from degradation. However, an understanding of the mechanism of action is not required to practice the technology provided.

5.1. Protein Composition

In addition to lipids (e.g., phosphatidylserine), exosomes contain cell-specific proteins that originate from the plasma membrane, cytosol, and intracellular endosomes. During the development of embodiments of the technology provided herein, experiments were conducted to examine the total protein contents of CD34+ and MNC exosomes and, in particular, to assess exosome marker proteins such as CD63, TSG101, and the CD34+ exosome-specific CD34 protein.

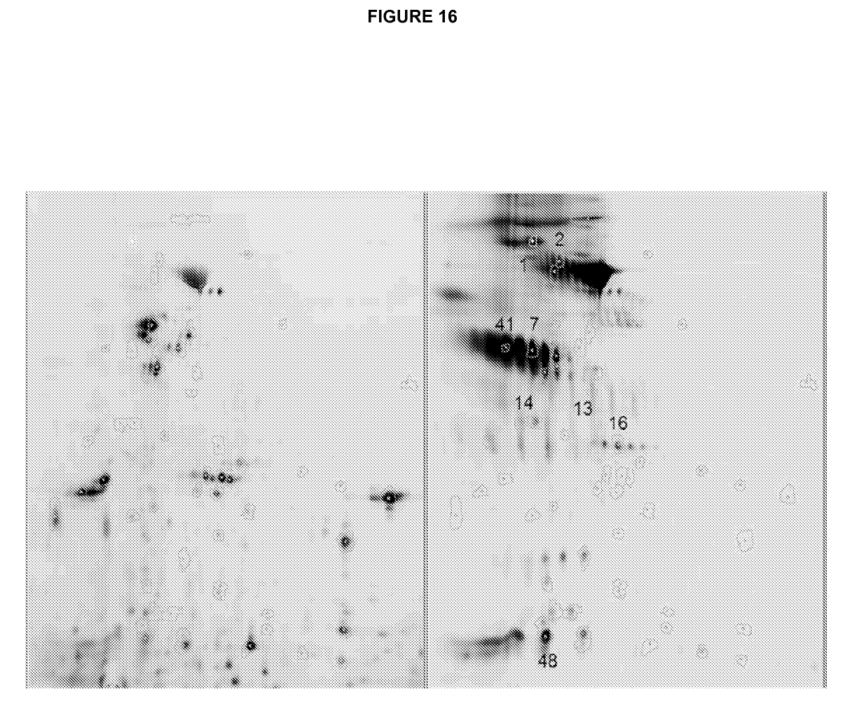

In addition, the proteins enriched in the CD34+ exosomes were identified by analyzing the total protein content of CD34+ and MNC exosomes by two-dimensional differential gel electrophoresis (DIGS). The two protein samples were labeled with two different fluorescent moieties, combined together, and separated by two-dimensional gel electrophoresis (FIG. 16). The different proteins were identified by relative differences in the fluorescence of the two labels and spots corresponding to the largest differences were picked using computer software as described below. Then, the proteins were identified by MS/MS analysis.

The MASCOT search engine (Matrix Science, www.matrixscience.com; see Electrophoresis 1999, 20(18): 3551-67) was used to identify proteins from primary sequence databases. The identified proteins are the best match for each sample. Proteins with Protein Score C.I. % or Total Ion C.I. % greater than 95 are considered high confidence matches. The best match was selected based on C.I. % and pI/MW location of the spot in the gel. The top ranked proteins and relative levels in the two samples are provided in Table 1.

TABLE 1
Proteins enriched in CD34+ exosomes

| | | CD34+ | | | | |
|---|---|---|---|---|---|---|
| | | Exo/ | | | | |
| | | MNC | Normoxia/ | Accession | | |
| Spot | Top Ranked Protein Name | Exo | Hypoxia | No. | MW | PI |
| 7 | haptoglobin | 123.87 | 1.00 | gi\|3337390 | 38209.2 | 6.1 |
| 41 | haptoglobin | 121.68 | −1.07 | gi\|3337390 | 38209.2 | 6.1 |
| 2 | hemopexin precursor | 48.84 | −1.09 | gi\|1321561 | 51643.3 | 6.6 |
| 1 | afamin precursor | 35.23 | −1.12 | gi\|4501987 | 69024.0 | 5.6 |
| 13 | haptoglobin isoform 2 preproprotein | 10.83 | −1.07 | gi\|186910296 | 38427.3 | 6.1 |
| 14 | complex-forming glycoprotein HC | 7.21 | 1.07 | gi\|223373 | 20421.2 | 5.8 |
| 33 | transthyretin precursor | 7.21 | 1.12 | gi\|4507725 | 15877.0 | 5.5 |
| 30 | haptoglobin Hp2 | 6.07 | −1.25 | gi\|223976 | 41716.9 | 6.2 |
| 53 | protein AMBP preproprotein | 5.93 | 1.00 | gi\|4502067 | 38974.0 | 6.0 |
| 16 | hemopexin, isoform CRA_c | 5.64 | 1.06 | gi\|119589126 | 28545.8 | 6.6 |
| 12 | PRO2675 | 4.44 | 1.32 | gi\|7770217 | 32553.4 | 6.1 |
| 48 | haptoglobin isoform 1 preproprotein | 2.75 | 1.10 | gi\|4826762 | 45176.6 | 6.1 |
| 34 | haptoglobin Hp2 | 2.54 | 1.08 | gi\|223976 | 41716.9 | 6.2 |
| 10 | alpha-enolase isoform 1 | 1.88 | 4.93 | gi\|4503571 | 47139.3 | 7.0 |
| 42 | glyceraldehyde-3-phosphate dehydrogenase | 1.34 | 2.17 | gi\|31645 | 36031.4 | 8.3 |
| 37 | hemopexin | −1.60 | −8.50 | gi\|226337 | 13337.6 | 6.7 |
| 11 | haptoglobin | −3.80 | 1.06 | gi\|1212947 | 38427.4 | 6.3 |
| 6 | hemopexin precursor | −4.64 | −1.14 | gi\|386789 | 51512.2 | 6.6 |
| 4 | transferrin | −19.73 | 2.55 | gi\|115394517 | 76909.6 | 7.0 |
| 38 | PRO2619 | -222.88 | −1.04 | gi\|11493459 | 56745.2 | 6.0 |

Two proteins that were enriched in CD34+ exosomes are haptoglobin and hemopexin. Haptoglobin is known as an angiogenic and anti-inflammatory molecule (see, e.g., Cid, M C, et. al. J. Clin. Invest. 1993, 91: 977-85) that acts by enhancing angiogenic and vasculogenic potential of EPCs (see, e.g., Park, S J, et al. FEBS Lett, 2009, 583: 3235-40), inducing anti-inflammatory and cytoprotective pathways by activating hemoglobin scavanger receptor CD163, releasing IL10, and activating heme oxygenase-1 synthesis (Philippidis, P. et al. Circ Res. 2004, 94: 119-26). Without being bound by theory, it is contemplated that this protein could be an important mediator of eliminating toxicity in the ischemic tissue and promoting angiogenesis; however, an understanding of the underlying mechanism is not required to practice the technology described herein. Further, under hypoxic conditions, haptoglobin expression is upregulated by hypoxia inducible factor-1α(HIF-1α) by a STAT-3 dependent pathway (Oh, M K. et al. J Biol Chem. 2011, 286: 8857-65), which reinforces its role under hypoxia and possibly in ischemia. Without being bound by theory, it is contemplated that hemopexin binds and scavenges free hemoglobin and protects the tissue from the oxidative damage that the free hemoglobin can cause. However, an understanding of the underlying mechanism is not required to practice the technology described herein. In certain embodiments, compositions comprising haptoglobin or hemopexin are used in the therapeutic technologies of the present disclosure (e.g., to promote angiogenesis).

5.2. RNA Composition

Experiments performed during the development of embodiments of the technology provided herein demonstrated that CD34+ exosomes carry several angiogenic miRNAs (Anand and Cheresh, Curr Opin Hematol, 2011, 3: 171; Fish & Srivastava, Sci Signal, 2009, 2(52) pe1) that are transferred to recipient endothelial cells.

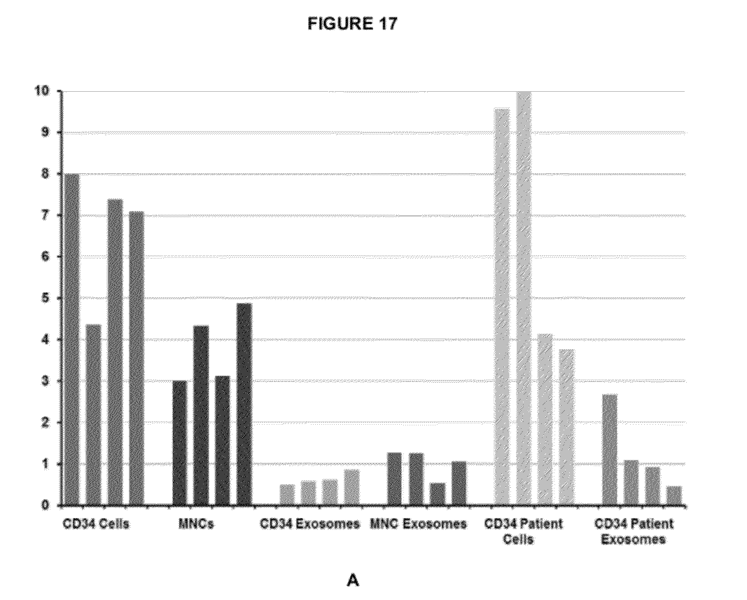

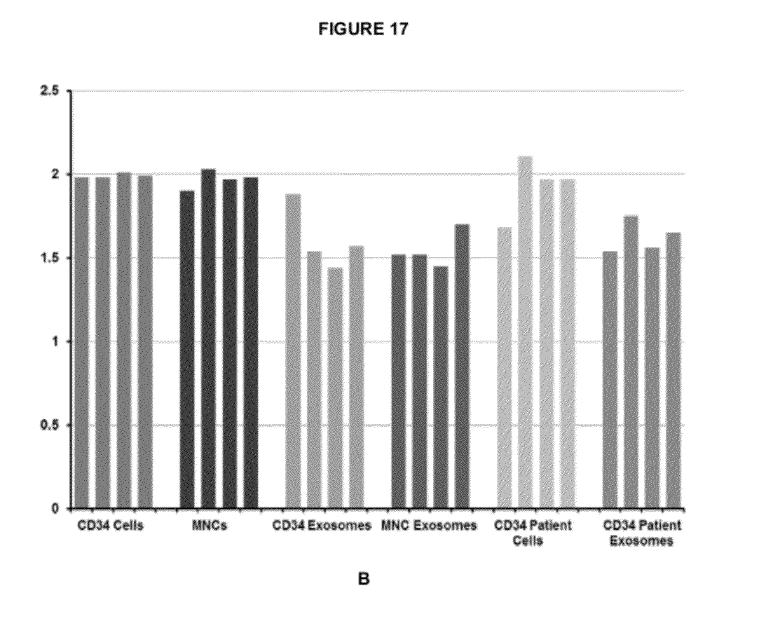

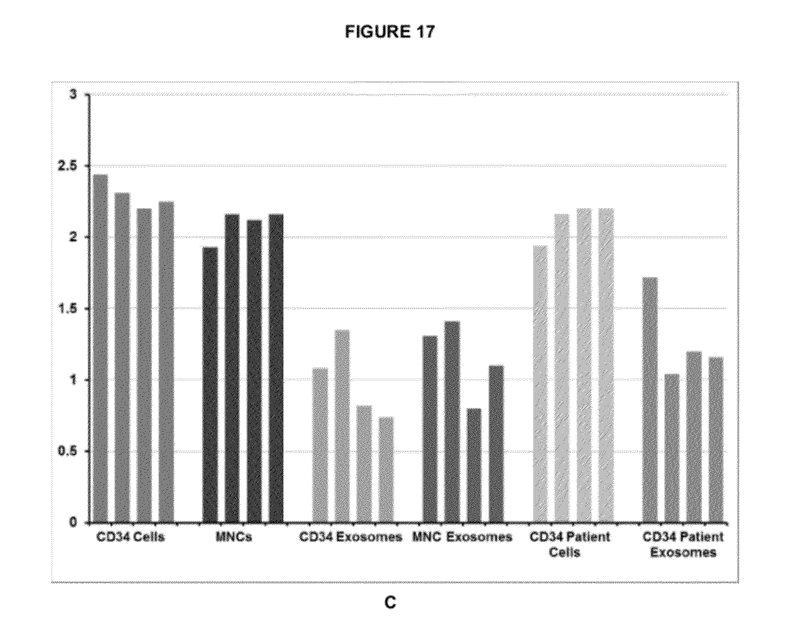

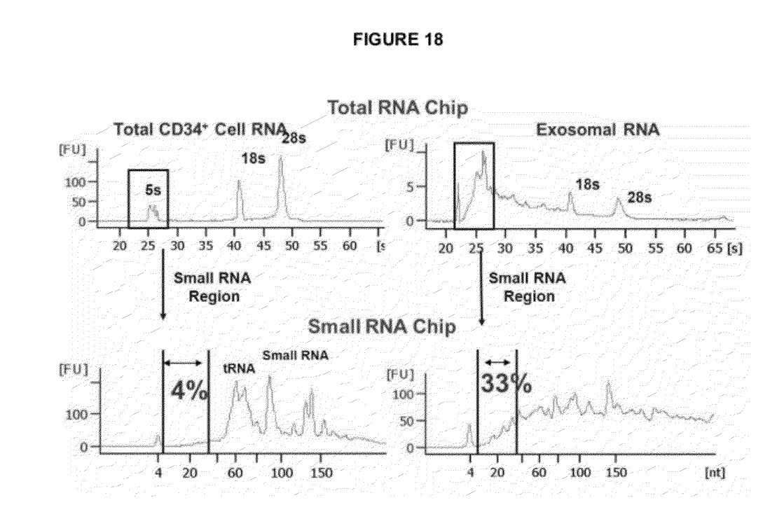

Total RNA was isolated from two functionally distinct exosomes: 1) CD34+ exosomes purified from adult human PB CD34+ cell culture conditioned media and 2) control exosomes from PB total MNC conditioned media. RNA was also isolated from critical limb ischemia patient PB CD34+ cells and exosomes and compared with healthy volunteer CD34+ cells and exosomes. Total RNA was quantified (FIG. 17a) and RNA quality was assessed by determining the ratios of the absorbance at 260 nm to the absorbance at 280 nm (FIG. 17b) and by determining the ratio of the absorbance at 260 nm to the absorbance at 230 nm (FIG. 17c). Total RNA isolated from exosomes was less than the total cellular RNA mostly because of the absence of the ribosomal RNA (FIG. 18).

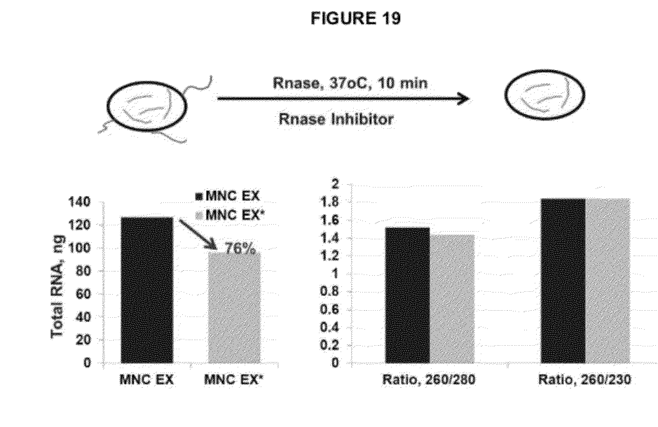

Analysis of the RNA samples for small RNAs indicates that exosomal RNA is enriched for small RNAs and miRNAs as compared to their cells of origin (33% in CD34+ exosomes versus 4% in CD34+ cells, FIG. 18, “Small RNA Chip”). A negative correlation between the miRNA percentage and total RNA integrity was found for all investigated samples. These data show that the CD34+ exosomes are enriched for small RNA species. It is contemplated that this specific packaging of exosomal RNA content might indicate the CD34+ exosome function in the target cells, though the technology is not bound to any particular theory and an understanding of the mechanism is not required to practice the technology. RNAse treatment of the exosome preparations did not significantly affect the quantity and quality of the RNA compared to exosomes that were not treated with RNAse (FIG. 19). Thus, most of the RNA isolated in the exosome samples was confirmed to be present inside the lumen of the exosomes.

Differential expression of miRNA between CD34+ and MNC exosomes was profiled using an Affymetrix miRNA microarray. The results (Table 2) show a significant increase in the expression of several pro-angiogenic miRNAs in the CD34+ cells as well as in the exosomes. For many of the pro-angiogenic miRNAs, the relative difference in the amounts of miRNA in the exosome samples (e.g., CD34+ exosomes compared to MNC exosomes) was higher than the relative difference in the amounts of miRNA in the cells from which the exosomes were prepared (e.g., CD34+ cells: MNCs) (Table 2). These data indicate that pro-angiogenic miRNAs are enriched in the CD34+ exosomes.

TABLE 2
Microarray results

| | CD34+/ | | CD34+ex/ | |
|---|---|---|---|---|
| | MNC | CD34+/ | MNCex | CD34+ex/ |
| | fold | MNC | fold | MNCex |
| ProbeSet Name | change | p-value | change | p-value |
| mmu-miR-92a_st | 3.395 | 0.01321 | 4.94 | 0.00002 |
| xtr-miR-92b_st | 8.711 | 0.00981 | 6.02 | 0.00004 |
| xla-miR-92a_st | 3.568 | 0.00515 | 4.87 | 0.00004 |
| U31_x_st | 0.761 | 0.09701 | 8.12 | 0.00007 |
| xtr-miR-181b_st | 2.559 | 0.00014 | 3.14 | 0.00014 |
| dse-miR-92a_st | 3.524 | 0.00009 | 4.36 | 0.00030 |
| tca-miR-92b_st | 3.630 | 0.01716 | 4.74 | 0.00035 |
| sla-miR-92_st | 3.227 | 0.00124 | 4.92 | 0.00039 |
| xtr-miR-92a_st | 3.835 | 0.01063 | 4.71 | 0.00058 |
| mdo-miR-92_st | 4.127 | 0.00509 | 4.40 | 0.00066 |
| bta-miR-2288_st | 1.948 | 0.07806 | 2.54 | 0.00072 |
| hsa-miR-92a_st | 3.871 | 0.00052 | 5.16 | 0.00084 |
| ame-miR-92a_st | 2.872 | 0.00196 | 5.84 | 0.00088 |
| dgr-miR-92b_st | 3.840 | 0.02537 | 5.67 | 0.00088 |
| spu-miR-92a_st | 3.398 | 0.00886 | 3.70 | 0.00090 |
| dps-miR-92a_st | 2.956 | 0.00649 | 4.34 | 0.00110 |
| aae-miR-92a_st | 4.265 | 0.00323 | 4.31 | 0.00114 |
| dwi-miR-92a_st | 3.287 | 0.00134 | 4.42 | 0.00115 |
| cqu-miR-92_st | 3.100 | 0.01652 | 5.07 | 0.00124 |
| ptr-miR-92_st | 3.257 | 0.00114 | 4.61 | 0.00129 |
| ssc-miR-181d_st | 8.303 | 0.01679 | 5.92 | 0.00142 |
| dre-miR-181b_st | 4.008 | 0.03570 | 2.88 | 0.00146 |
| rno-miR-181b_st | 2.476 | 0.01495 | 3.61 | 0.00153 |
| dsi-miR-92a_st | 3.147 | 0.00427 | 4.69 | 0.00154 |
| tni-miR-92_st | 2.584 | 0.01892 | 4.76 | 0.00165 |
| dme-miR-92a_st | 3.077 | 0.02445 | 5.15 | 0.00166 |
| lgi-miR-92_st | 3.130 | 0.02005 | 4.71 | 0.00176 |
| sko-miR-92a_st | 2.975 | 0.00513 | 5.29 | 0.00189 |
| spu-miR-92c_st | 5.265 | 0.00507 | 5.33 | 0.00191 |
| U76_st | 1.046 | 0.83119 | 16.92 | 0.00194 |
| cfa-miR-92a_st | 3.268 | 0.00462 | 4.57 | 0.00197 |
| eca-miR-92a_st | 2.925 | 0.00953 | 4.82 | 0.00206 |
| dpe-miR-92a_st | 3.362 | 0.00637 | 5.32 | 0.00215 |
| lca-miR-92_st | 3.231 | 0.00243 | 4.92 | 0.00216 |
| hp_hsa-mir-524_st | 1.214 | 0.52238 | 2.94 | 0.00216 |
| cin-miR-92a_st | 2.782 | 0.02226 | 6.29 | 0.00230 |
| bma-miR-92_st | 4.172 | 0.02486 | 5.34 | 0.00242 |
| dre-miR-92b_st | 5.059 | 0.02000 | 9.38 | 0.00269 |
| rno-miR-92b_st | 4.735 | 0.01479 | 5.20 | 0.00281 |
| aga-miR-92b_st | 2.661 | 0.01638 | 4.07 | 0.00282 |
| dmo-miR-92b_st | 3.519 | 0.01732 | 5.24 | 0.00283 |
| aga-miR-92a_st | 2.900 | 0.01209 | 4.76 | 0.00297 |
| ACA30_x_st | 0.898 | 0.17724 | 2.19 | 0.00308 |
| jcv-miR-J1-5p_st | 1.880 | 0.13077 | 2.12 | 0.00311 |
| ACA58_st | 1.907 | 0.11458 | 4.89 | 0.00312 |
| dvi-miR-92a_st | 3.154 | 0.01093 | 4.91 | 0.00337 |
| aae-miR-92b_st | 3.174 | 0.00505 | 4.50 | 0.00342 |
| bta-miR-92_st | 3.330 | 0.01473 | 4.62 | 0.00344 |
| eca-miR-181b_st | 2.936 | 0.06335 | 2.55 | 0.00344 |
| tni-miR-181b_st | 2.819 | 0.01860 | 3.70 | 0.00346 |
| mmu-let-7d_st | 1.162 | 0.53024 | 3.06 | 0.00346 |
| bfl-miR-92b_st | 4.542 | 0.01012 | 5.97 | 0.00371 |
| tgu-miR-181b_st | 4.477 | 0.01062 | 2.51 | 0.00372 |
| gga-miR-92_st | 3.258 | 0.00597 | 5.58 | 0.00381 |
| mml-miR-92b_st | 5.003 | 0.03881 | 5.27 | 0.00382 |
| dwi-miR-92b_st | 4.759 | 0.00865 | 4.58 | 0.00382 |
| ENSG00000252213_x_st | 3.370 | 0.22051 | 9.25 | 0.00384 |
| gga-let-7c_st | 1.253 | 0.28206 | 3.55 | 0.00406 |
| dpu-miR-92_st | 5.202 | 0.02878 | 6.33 | 0.00412 |
| ppy-miR-181a_st | 2.285 | 0.02916 | 2.45 | 0.00426 |
| oan-miR-92a_st | 3.064 | 0.02823 | 4.22 | 0.00435 |
| lla-miR-92_st | 3.026 | 0.00620 | 4.22 | 0.00437 |
| dan-miR-92a_st | 4.057 | 0.00833 | 4.54 | 0.00440 |
| bfl-miR-92a_st | 3.825 | 0.00683 | 4.72 | 0.00471 |
| U29_st | 1.081 | 0.55666 | 8.70 | 0.00491 |
| dmo-miR-92a_st | 3.416 | 0.00459 | 4.05 | 0.00492 |
| bmo-miR-92b_st | 8.020 | 0.11281 | 14.67 | 0.00514 |
| dre-miR-92a_st | 3.605 | 0.00715 | 4.49 | 0.00522 |
| oan-miR-92b_st | 3.792 | 0.01056 | 7.95 | 0.00526 |
| spu-miR-92b_st | 3.843 | 0.01922 | 4.38 | 0.00545 |
| cte-miR-92a_st | 2.554 | 0.01809 | 6.48 | 0.00561 |
| ggo-miR-92_st | 2.971 | 0.01694 | 4.68 | 0.00583 |
| oan-miR-181b_st | 2.899 | 0.01822 | 2.92 | 0.00594 |
| ppy-miR-92_st | 3.215 | 0.00159 | 5.42 | 0.00602 |
| dgr-miR-92a_st | 3.747 | 0.00044 | 4.56 | 0.00608 |
| csa-miR-92c_st | 3.519 | 0.02634 | 6.21 | 0.00621 |
| bfl-miR-92c_st | 4.265 | 0.01792 | 5.65 | 0.00621 |
| sko-miR-92c_st | 3.205 | 0.01040 | 6.96 | 0.00623 |
| ppy-miR-1246_st | 1.361 | 0.10668 | 2.10 | 0.00629 |
| mne-miR-92_st | 3.199 | 0.00027 | 5.39 | 0.00633 |
| dya-miR-92a_st | 4.042 | 0.00371 | 5.20 | 0.00660 |
| rno-miR-92a_st | 4.585 | 0.00157 | 4.75 | 0.00677 |
| hsa-miR-181a_st | 2.553 | 0.02956 | 2.75 | 0.00686 |
| bta-miR-92a_st | 3.005 | 0.01487 | 4.77 | 0.00691 |
| dya-miR-92b_st | 4.775 | 0.01330 | 5.77 | 0.00711 |
| dpe-miR-92b_st | 4.295 | 0.00728 | 6.59 | 0.00722 |
| ssc-miR-92a_st | 3.738 | 0.00294 | 5.90 | 0.00729 |
| HBII-95_x_st | 1.164 | 0.39571 | 4.05 | 0.00777 |
| fru-miR-92_st | 3.703 | 0.02075 | 4.92 | 0.00781 |
| der-miR-92a_st | 3.848 | 0.00318 | 5.96 | 0.00783 |
| ppy-miR-181b_st | 2.451 | 0.11174 | 3.24 | 0.00784 |
| U33_st | 0.761 | 0.14169 | 8.68 | 0.00789 |
| mml-miR-486-5p_st | 25.285 | 0.04654 | 15.90 | 0.00797 |
| dvi-miR-92b_st | 3.894 | 0.00063 | 5.61 | 0.00800 |
| HBI-115_st | 11.446 | 0.01072 | 5.71 | 0.00819 |
| tgu-miR-92_st | 2.893 | 0.04217 | 4.62 | 0.00822 |
| mml-miR-92a_st | 3.630 | 0.00918 | 5.07 | 0.00827 |
| dse-miR-92b_st | 5.525 | 0.01438 | 5.00 | 0.00837 |
| dre-miR-181c_st | 4.518 | 0.03314 | 3.15 | 0.00849 |
| odi-miR-92a_st | 4.112 | 0.02141 | 7.35 | 0.00897 |
| cin-let-7b_st | 1.836 | 0.21189 | 2.33 | 0.00933 |
| bta-miR-181b_st | 3.212 | 0.03512 | 2.23 | 0.00934 |
| dsi-miR-310_st | 1.176 | 0.46399 | 2.04 | 0.00940 |
| hsa-miR-2115-star_st | 1.806 | 0.20625 | 2.14 | 0.00961 |
| nvi-miR-92a_st | 3.498 | 0.00223 | 5.16 | 0.00965 |
| gga-let-7b_st | 1.666 | 0.18637 | 2.50 | 0.00966 |
| U58C_x_st | 1.408 | 0.29962 | 19.14 | 0.00977 |
| cin-miR-92d-3p_st | 4.994 | 0.04974 | 5.69 | 0.01016 |
| dsi-miR-92b_st | 3.920 | 0.02132 | 5.16 | 0.01027 |
| U34_st | 0.855 | 0.20539 | 5.73 | 0.01036 |
| dps-miR-92b_st | 4.041 | 0.01327 | 5.94 | 0.01040 |
| SNORA38B_st | 1.233 | 0.14887 | 6.20 | 0.01069 |
| tni-let-7h_st | 2.439 | 0.29043 | 4.32 | 0.01094 |
| gga-miR-181b_st | 2.634 | 0.01655 | 3.58 | 0.01111 |
| hsa-miR-92b_st | 4.611 | 0.00826 | 8.88 | 0.01223 |
| ppa-miR-92_st | 2.904 | 0.00620 | 4.56 | 0.01241 |
| lla-miR-181a_st | 2.546 | 0.03760 | 2.56 | 0.01262 |
| Z17B_st | 1.338 | 0.24215 | 3.91 | 0.01304 |
| tni-miR-181a_st | 2.417 | 0.02534 | 2.53 | 0.01308 |
| U101_st | 1.145 | 0.36744 | 4.09 | 0.01332 |
| U54_st | 1.267 | 0.32872 | 4.31 | 0.01363 |
| eca-miR-92b_st | 4.537 | 0.01336 | 6.65 | 0.01375 |
| ssc-miR-181b_st | 2.778 | 0.02027 | 3.07 | 0.01403 |
| csa-miR-92b_st | 4.997 | 0.04282 | 7.21 | 0.01467 |
| mmu-let-7b_st | 1.319 | 0.04672 | 2.72 | 0.01473 |
| hsa-miR-181b_st | 2.508 | 0.04232 | 4.13 | 0.01478 |
| dya-miR-125_st | 11.454 | 0.00528 | 137.98 | 0.01512 |
| cte-miR-125_st | 16.881 | 0.00332 | 98.32 | 0.01549 |
| cte-miR-92c_st | 3.719 | 0.00596 | 4.45 | 0.01581 |
| lla-miR-125b_st | 31.919 | 0.00556 | 82.15 | 0.01581 |
| ACA57_st | 1.332 | 0.07723 | 7.30 | 0.01584 |
| fru-let-7a_st | 1.269 | 0.14004 | 2.56 | 0.01592 |
| cfa-miR-92b_st | 3.882 | 0.00986 | 6.66 | 0.01616 |
| eca-miR-1291b_st | 3.420 | 0.49212 | 3.45 | 0.01621 |
| bfl-miR-125_st | 18.222 | 0.00118 | 202.15 | 0.01622 |
| hp_mmu-mir-106a_st | 1.824 | 0.46483 | 2.87 | 0.01667 |
| mmu-miR-181a_st | 1.871 | 0.09656 | 2.52 | 0.01691 |
| ptr-let-7c_st | 1.451 | 0.28561 | 3.27 | 0.01722 |
| oan-let-7b_st | 1.595 | 0.05799 | 2.75 | 0.01723 |
| sko-miR-92b_st | 3.460 | 0.02006 | 3.97 | 0.01756 |
| hsa-miR-125b_st | 13.362 | 0.00264 | 184.34 | 0.01776 |
| U49A_x_st | 1.194 | 0.43980 | 8.37 | 0.01783 |
| ENSG00000252765_x_st | 1.045 | 0.93252 | 2.31 | 0.01789 |
| cbr-miR-235_st | 2.412 | 0.04070 | 2.48 | 0.01799 |
| dan-miR-92b_st | 3.318 | 0.02271 | 8.87 | 0.01839 |
| ptr-let-7b_st | 1.492 | 0.06271 | 3.22 | 0.01855 |
| HBII-316_st | 1.633 | 0.29255 | 10.69 | 0.01889 |
| dme-miR-92b_st | 4.958 | 0.01763 | 6.99 | 0.01925 |
| bta-miR-92b_st | 4.262 | 0.03351 | 9.06 | 0.01932 |
| mdo-miR-181b_st | 3.314 | 0.01040 | 2.96 | 0.01967 |
| ggo-miR-125b_st | 14.988 | 0.00084 | 173.49 | 0.01974 |
| ppy-miR-92b_st | 4.045 | 0.01909 | 8.41 | 0.01979 |
| tni-miR-125b_st | 24.257 | 0.01214 | 176.36 | 0.02012 |
| age-miR-92_st | 2.516 | 0.03259 | 5.00 | 0.02063 |
| ACA35_st | 2.468 | 0.25722 | 3.93 | 0.02109 |
| mgh28S-2409_x_st | 0.930 | 0.58091 | 7.32 | 0.02116 |
| mml-let-7b_st | 1.428 | 0.05808 | 3.05 | 0.02138 |
| cfa-let-7b_st | 1.444 | 0.10579 | 3.10 | 0.02145 |
| bmo-miR-92a_st | 6.099 | 0.08268 | 3.17 | 0.02167 |
| oan-miR-92c_st | 8.545 | 0.06106 | 7.53 | 0.02212 |
| dpe-miR-125_st | 11.057 | 0.00337 | 189.22 | 0.02223 |
| ssc-miR-181a_st | 2.908 | 0.05425 | 2.45 | 0.02263 |
| U50B_x_st | 0.652 | 0.03914 | 2.97 | 0.02285 |
| cin-miR-92c_st | 3.616 | 0.02198 | 3.38 | 0.02290 |
| U58B_x_st | 0.980 | 0.93974 | 9.03 | 0.02298 |
| bta-miR-181a_st | 2.632 | 0.05675 | 2.30 | 0.02313 |
| HBII-55_st | 0.806 | 0.28908 | 10.78 | 0.02316 |
| tni-miR-130_st | 46.492 | 0.00867 | 42.08 | 0.02325 |
| dgr-miR-125_st | 16.459 | 0.01081 | 205.95 | 0.02352 |
| mdo-let-7b_st | 1.690 | 0.03527 | 2.11 | 0.02382 |
| nvi-let-7_st | 1.359 | 0.03156 | 2.93 | 0.02383 |
| U62A_s_st | 1.342 | 0.50282 | 11.91 | 0.02384 |
| mmu-miR-125b-5p_st | 18.700 | 0.01353 | 121.92 | 0.02409 |
| U50_st | 0.662 | 0.30233 | 3.31 | 0.02443 |
| hp_hsa-mir-222_st | 2.363 | 0.06161 | 3.63 | 0.02449 |
| ppa-miR-181a_st | 2.120 | 0.03209 | 2.15 | 0.02456 |
| der-miR-92b_st | 3.842 | 0.00771 | 5.12 | 0.02480 |
| rno-let-7b_st | 1.565 | 0.16797 | 3.02 | 0.02501 |
| ppa-miR-125b_st | 13.316 | 0.00248 | 112.07 | 0.02504 |
| dme-let-7_st | 1.376 | 0.03361 | 2.51 | 0.02505 |
| ggo-miR-181a_st | 2.373 | 0.03386 | 2.28 | 0.02531 |
| mml-miR-361-5p_st | 1.125 | 0.46961 | 2.77 | 0.02565 |
| xtr-let-7c_st | 1.314 | 0.13840 | 2.37 | 0.02565 |
| dre-let-7d_st | 3.698 | 0.05309 | 5.31 | 0.02591 |
| eca-miR-17_st | 5.038 | 0.00383 | 6.18 | 0.02604 |
| mdo-miR-181a_st | 1.991 | 0.05802 | 2.33 | 0.02614 |
| ACA14b_x_st | 1.650 | 0.22424 | 3.53 | 0.02638 |
| bta-let-7b_st | 1.404 | 0.04678 | 2.02 | 0.02667 |
| ppy-let-7c_st | 1.388 | 0.16137 | 2.85 | 0.02702 |
| aae-miR-125-star_st | 18.313 | 0.00133 | 129.57 | 0.02704 |
| HBII-289_st | 0.357 | 0.15021 | 6.30 | 0.02777 |
| xtr-miR-181a_st | 2.541 | 0.00943 | 2.68 | 0.02781 |
| bma-let-7_st | 1.364 | 0.00147 | 2.94 | 0.02786 |
| dsi-miR-125_st | 12.365 | 0.00115 | 121.96 | 0.02804 |
| ACA40_x_st | 1.850 | 0.20991 | 9.75 | 0.02813 |
| HBII-276_st | 1.020 | 0.94128 | 7.87 | 0.02824 |
| lla-miR-181b_st | 3.547 | 0.06382 | 3.03 | 0.02828 |
| mmu-miR-181b_st | 2.465 | 0.09794 | 2.55 | 0.02847 |
| U31_st | 0.760 | 0.05946 | 6.51 | 0.02868 |
| dme-miR-125_st | 12.916 | 0.00085 | 132.03 | 0.02889 |
| dps-miR-92c_st | 3.999 | 0.05690 | 2.25 | 0.02904 |
| mmu-miR-181d_st | 10.300 | 0.04804 | 5.88 | 0.02910 |
| cfa-miR-181b_st | 5.007 | 0.10375 | 4.81 | 0.02913 |
| mmu-let-7e_st | 1.106 | 0.81760 | 5.01 | 0.02937 |
| dvi-miR-125_st | 27.713 | 0.01472 | 232.14 | 0.02962 |
| gga-let-7j_st | 1.269 | 0.10021 | 3.02 | 0.02968 |
| U51_st | 1.304 | 0.12509 | 5.45 | 0.02985 |
| tgu-miR-125_st | 14.076 | 0.00106 | 173.51 | 0.02998 |
| bta-miR-130a_st | 56.869 | 0.00392 | 38.52 | 0.03016 |
| eca-miR-181a_st | 2.066 | 0.08200 | 2.44 | 0.03026 |
| ssc-let-7c_st | 1.420 | 0.25531 | 2.54 | 0.03045 |
| cfa-miR-181d_st | 6.946 | 0.05943 | 4.60 | 0.03078 |
| rno-miR-181d_st | 7.530 | 0.02094 | 5.96 | 0.03145 |
| U17b_x_st | 2.487 | 0.22769 | 4.04 | 0.03171 |
| mml-miR-17-5p_st | 4.158 | 0.01881 | 8.24 | 0.03189 |
| dps-miR-125_st | 32.964 | 0.00830 | 162.38 | 0.03214 |
| U58A_x_st | 1.182 | 0.56517 | 7.48 | 0.03218 |
| U75_x_st | 1.187 | 0.49298 | 3.76 | 0.03220 |
| gga-miR-17-5p_st | 4.389 | 0.00502 | 6.43 | 0.03222 |
| gga-miR-20b_st | 6.076 | 0.03959 | 6.76 | 0.03235 |
| ame-miR-125_st | 20.839 | 0.01639 | 171.67 | 0.03235 |
| tca-miR-92a_st | 4.777 | 0.02700 | 7.22 | 0.03255 |
| mml-miR-181c_st | 4.348 | 0.16229 | 6.86 | 0.03359 |
| U50B_st | 0.611 | 0.07102 | 6.50 | 0.03378 |
| ppa-miR-130a_st | 53.807 | 0.00452 | 31.67 | 0.03410 |
| dre-miR-125b_st | 20.570 | 0.01951 | 115.23 | 0.03419 |
| mml-miR-181b_st | 3.762 | 0.02295 | 2.31 | 0.03423 |
| hsa-miR-20b_st | 4.766 | 0.04312 | 7.60 | 0.03443 |
| ppy-miR-130a_st | 111.938 | 0.00028 | 35.14 | 0.03478 |
| oan-miR-23a-star_st | 0.983 | 0.98117 | 2.46 | 0.03478 |
| HBII-85-6_x_st | 1.650 | 0.07879 | 5.63 | 0.03484 |
| eca-let-7d_st | 1.060 | 0.59167 | 3.73 | 0.03492 |
| ssc-miR-17-5p_st | 4.781 | 0.00795 | 7.20 | 0.03511 |
| ppy-miR-125b_st | 20.133 | 0.02089 | 167.55 | 0.03554 |
| U36C_st | 1.194 | 0.42845 | 4.86 | 0.03601 |
| tni-let-7b_st | 1.215 | 0.33935 | 2.62 | 0.03603 |
| cfa-miR-181c_st | 4.739 | 0.09259 | 5.96 | 0.03612 |
| ACA18_x_st | 1.833 | 0.13736 | 11.80 | 0.03642 |
| sla-miR-181a_st | 1.800 | 0.04146 | 2.57 | 0.03722 |
| dan-miR-125_st | 16.324 | 0.01727 | 153.99 | 0.03827 |
| hsa-let-7c_st | 1.334 | 0.32936 | 2.61 | 0.03845 |
| ptr-miR-130a_st | 45.522 | 0.00068 | 32.64 | 0.03875 |
| crm-miR-235_st | 6.128 | 0.04356 | 17.31 | 0.03895 |
| cin-miR-92b_st | 2.911 | 0.01217 | 4.07 | 0.03918 |
| tgu-miR-20a_st | 4.825 | 0.01648 | 6.78 | 0.03921 |
| ssc-miR-20_st | 3.923 | 0.00788 | 7.15 | 0.03939 |
| hsa-miR-181d_st | 6.466 | 0.06411 | 3.90 | 0.04011 |
| mmu-miR-92b_st | 3.752 | 0.00990 | 6.67 | 0.04031 |
| dan-miR-311a_st | 2.513 | 0.21894 | 3.70 | 0.04033 |
| xtr-miR-17-5p_st | 4.631 | 0.00972 | 5.65 | 0.04042 |
| gga-miR-130a_st | 60.251 | 0.01082 | 33.08 | 0.04043 |
| xla-miR-20_st | 6.628 | 0.02418 | 8.38 | 0.04051 |
| U23_st | 1.987 | 0.30687 | 4.44 | 0.04056 |
| mmu-miR-20b_st | 5.357 | 0.03805 | 5.69 | 0.04076 |
| hsa-miR-130a_st | 41.810 | 0.00162 | 37.84 | 0.04084 |
| ppy-let-7a_st | 1.416 | 0.04864 | 2.93 | 0.04101 |
| csa-miR-92a_st | 3.705 | 0.03520 | 6.10 | 0.04125 |
| mmu-miR-17_st | 4.305 | 0.00231 | 6.80 | 0.04145 |
| gga-let-7a_st | 1.092 | 0.43450 | 2.81 | 0.04189 |
| xtr-let-7a_st | 1.332 | 0.11817 | 2.82 | 0.04190 |
| U81_x_st | 0.898 | 0.52674 | 13.27 | 0.04218 |
| ppy-let-7d_st | 1.155 | 0.18837 | 2.71 | 0.04227 |
| HBII-13_x_st | 0.547 | 0.06825 | 3.11 | 0.04229 |
| oan-miR-17_st | 4.067 | 0.00209 | 8.75 | 0.04251 |
| mne-miR-125b_st | 12.582 | 0.00578 | 117.36 | 0.04267 |
| bta-let-7c_st | 1.135 | 0.46056 | 2.94 | 0.04279 |
| oan-miR-221_st | 1.938 | 0.01090 | 3.73 | 0.04289 |
| hsa-let-7b_st | 1.403 | 0.14846 | 2.69 | 0.04313 |
| U104_st | 0.976 | 0.54150 | 3.13 | 0.04345 |
| dre-miR-181a_st | 2.225 | 0.01675 | 2.46 | 0.04346 |
| mmu-let-7a_st | 1.238 | 0.23064 | 2.97 | 0.04363 |
| mmu-let-7c_st | 1.431 | 0.15512 | 3.07 | 0.04364 |
| ssc-miR-92b_st | 4.308 | 0.02123 | 5.99 | 0.04375 |
| cfa-miR-106a_st | 4.615 | 0.00091 | 6.98 | 0.04381 |
| eca-let-7c_st | 1.369 | 0.13543 | 2.39 | 0.04383 |
| ptr-miR-125b_st | 15.190 | 0.00500 | 139.67 | 0.04397 |
| mdo-miR-106_st | 4.647 | 0.00532 | 6.85 | 0.04409 |
| xtr-miR-222_st | 1.662 | 0.08341 | 3.32 | 0.04431 |
| hsa-miR-106a_st | 4.650 | 0.00480 | 6.22 | 0.04435 |
| der-miR-125_st | 21.255 | 0.00592 | 127.58 | 0.04440 |
| U94_st | 0.954 | 0.91991 | 7.41 | 0.04463 |
| age-miR-125b_st | 15.088 | 0.00108 | 168.28 | 0.04471 |
| U30_st | 1.136 | 0.52509 | 7.51 | 0.04475 |
| ppy-miR-17-5p_st | 5.224 | 0.00162 | 6.71 | 0.04484 |
| mgh28S-2411_st | 0.971 | 0.91049 | 8.42 | 0.04506 |
| dre-miR-130a_st | 32.689 | 0.00633 | 49.98 | 0.04542 |
| bta-miR-17-5p_st | 5.039 | 0.00999 | 7.30 | 0.04543 |
| ggo-miR-130a_st | 62.415 | 0.00301 | 65.30 | 0.04552 |
| mdo-miR-125b_st | 13.108 | 0.00085 | 126.23 | 0.04562 |
| HBII-99_st | 1.850 | 0.02812 | 10.52 | 0.04613 |
| HBII-295_st | 0.843 | 0.53757 | 5.09 | 0.04634 |
| oan-miR-181a_st | 2.448 | 0.02697 | 2.51 | 0.04649 |
| U38A_st | 0.813 | 0.56816 | 4.81 | 0.04691 |
| dre-miR-20b_st | 5.932 | 0.00389 | 9.99 | 0.04753 |
| fru-miR-125b_st | 16.310 | 0.00139 | 243.59 | 0.04755 |
| ACA2b_st | 1.179 | 0.60115 | 3.98 | 0.04796 |
| lca-miR-125b_st | 12.266 | 0.02183 | 112.06 | 0.04802 |
| xtr-miR-106_st | 4.870 | 0.00687 | 8.41 | 0.04833 |
| mne-miR-130a_st | 42.588 | 0.00026 | 27.74 | 0.04892 |
| mne-miR-17-5p_st | 4.414 | 0.00649 | 6.92 | 0.04903 |
| xtr-miR-20b_st | 5.226 | 0.03051 | 6.32 | 0.04919 |
| ppa-miR-106a_st | 4.335 | 0.00413 | 6.71 | 0.04922 |
| ptr-miR-181a_st | 2.201 | 0.03174 | 2.42 | 0.04929 |
| tgu-let-7d_st | 1.126 | 0.52359 | 3.45 | 0.04929 |
| ACA3-2_x_st | 0.769 | 0.35516 | 9.53 | 0.04930 |
| oan-miR-125_st | 12.043 | 0.00719 | 119.98 | 0.04957 |
| HBI-61_x_st | 1.172 | 0.67476 | 13.39 | 0.04962 |
| ENSG00000238956_s_st | 0.846 | 0.11845 | 9.45 | 0.04967 |
| tgu-miR-181a_st | 2.031 | 0.05808 | 2.16 | 0.04967 |
| sla-miR-106a_st | 5.287 | 0.00941 | 6.70 | 0.04969 |
| U46_s_st | 1.053 | 0.78482 | 10.83 | 0.04977 |
| ppy-miR-25_st | 1.640 | 0.00578 | 2.71 | 0.04977 |
| dgr-let-7_st | 1.020 | 0.83215 | 2.56 | 0.04983 |
| dvi-let-7_st | 1.193 | 0.07667 | 2.83 | 0.04985 |
| fru-miR-181a_st | 1.833 | 0.04877 | 2.38 | 0.04988 |
| mml-let-7d_st | 1.180 | 0.51491 | 2.98 | 0.04990 |
| mne-miR-181b_st | 3.197 | 0.04686 | 3.11 | 0.04992 |
| U28_x_st | 1.430 | 0.50491 | 5.39 | 0.05026 |
| tni-miR-25_st | 1.678 | 0.03208 | 3.09 | 0.05035 |
| sko-miR-125_st | 13.767 | 0.00239 | 185.42 | 0.05106 |
| ACA34_x_st | 2.011 | 0.29384 | 9.95 | 0.05116 |
| dre-miR-125c_st | 22.749 | 0.01126 | 114.17 | 0.05127 |
| tni-let-7a_st | 1.414 | 0.03282 | 2.35 | 0.05131 |
| dre-miR-130c_st | 50.779 | 0.01653 | 28.17 | 0.05137 |
| mmu-miR-181c_st | 2.184 | 0.33264 | 4.51 | 0.05161 |
| hp_hsa-mir-1248_s_st | 2.007 | 0.39911 | 6.84 | 0.05162 |
| SNORD127_st | 1.003 | 0.99378 | 2.08 | 0.05168 |
| ggo-miR-181c_st | 3.075 | 0.21935 | 4.10 | 0.05170 |
| hsa-miR-17_st | 4.214 | 0.00146 | 5.60 | 0.05186 |
| spu-let-7_st | 1.278 | 0.23947 | 3.01 | 0.05211 |
| rno-miR-125b-5p_st | 16.188 | 0.00910 | 136.94 | 0.05243 |
| cfa-miR-20a_st | 3.929 | 0.01593 | 13.19 | 0.05249 |
| dse-miR-125_st | 17.286 | 0.01764 | 138.31 | 0.05257 |
| ggo-miR-20_st | 4.338 | 0.00949 | 8.00 | 0.05264 |
| dre-let-7c_st | 1.220 | 0.41494 | 2.68 | 0.05269 |
| rno-miR-130a_st | 39.791 | 0.00036 | 43.10 | 0.05277 |
| oan-miR-20b_st | 4.316 | 0.02685 | 8.16 | 0.05281 |
| mml-miR-125b_st | 19.971 | 0.00064 | 73.74 | 0.05287 |
| tni-miR-20_st | 4.418 | 0.01210 | 9.32 | 0.05301 |
| eca-miR-20b_st | 6.020 | 0.03611 | 10.60 | 0.05316 |
| cfa-miR-127_st | 1.003 | 0.99440 | 8.38 | 0.05319 |
| ACA2b_x_st | 1.099 | 0.73497 | 2.34 | 0.05321 |
| gga-miR-106_st | 4.049 | 0.01217 | 6.91 | 0.05332 |
| U27_x_st | 0.683 | 0.02488 | 8.87 | 0.05341 |
| dya-let-7_st | 1.171 | 0.03010 | 3.32 | 0.05357 |
| age-miR-20_st | 4.178 | 0.01366 | 8.10 | 0.05362 |
| mne-miR-106a_st | 4.337 | 0.00931 | 5.74 | 0.05374 |
| ptr-miR-181d_st | 6.369 | 0.05758 | 5.44 | 0.05389 |
| bta-miR-106_st | 4.025 | 0.00277 | 7.94 | 0.05415 |
| odi-let-7d_st | 1.570 | 0.28430 | 2.79 | 0.05419 |
| ACA44_s_st | 1.210 | 0.64248 | 20.55 | 0.05426 |
| dan-miR-100_st | 4.634 | 0.33777 | 2.64 | 0.05430 |
| ENSG00000207118_st | 0.927 | 0.79737 | 4.10 | 0.05434 |
| ptr-miR-20a_st | 4.341 | 0.01962 | 7.31 | 0.05470 |
| ACA44_st | 1.727 | 0.51450 | 60.39 | 0.05471 |
| mdo-miR-130a_st | 43.301 | 0.00413 | 42.49 | 0.05471 |
| mml-miR-106a_st | 4.317 | 0.00708 | 7.56 | 0.05491 |
| fru-miR-222_st | 1.627 | 0.14515 | 2.66 | 0.05533 |
| cfa-let-7a_st | 1.178 | 0.10580 | 2.81 | 0.05544 |
| ppy-miR-106a_st | 4.318 | 0.00390 | 5.97 | 0.05550 |
| U26_st | 0.949 | 0.64108 | 4.10 | 0.05569 |
| fru-miR-20_st | 4.091 | 0.01398 | 6.62 | 0.05575 |
| dre-let-7g_st | 0.944 | 0.72443 | 4.90 | 0.05576 |
| U19_st | 3.571 | 0.02641 | 12.81 | 0.05581 |
| ACA13_st | 1.984 | 0.09370 | 4.94 | 0.05587 |
| cfa-let-7e_st | 1.049 | 0.91672 | 3.43 | 0.05593 |
| hp_hsa-mir-92b_st | 2.185 | 0.06183 | 2.49 | 0.05618 |
| dre-miR-17a_st | 4.764 | 0.00244 | 7.26 | 0.05621 |
| dre-miR-20a_st | 5.589 | 0.00734 | 9.27 | 0.05621 |
| xtr-let-7e_st | 2.054 | 0.13768 | 2.14 | 0.05626 |
| U63_st | 0.752 | 0.02906 | 5.18 | 0.05655 |
| mdo-let-7d_st | 1.124 | 0.50769 | 2.23 | 0.05687 |
| xtr-miR-125b_st | 16.464 | 0.00032 | 97.84 | 0.05709 |
| xtr-miR-20a_st | 4.447 | 0.00749 | 8.60 | 0.05726 |
| bta-miR-125b_st | 20.363 | 0.00219 | 153.42 | 0.05732 |
| mml-let-7e_st | 1.111 | 0.84202 | 3.10 | 0.05738 |
| U93_st | 2.167 | 0.13692 | 3.52 | 0.05746 |
| bta-let-7a_st | 1.194 | 0.25237 | 2.53 | 0.05762 |
| dse-let-7_st | 1.140 | 0.01718 | 2.59 | 0.05779 |
| rno-miR-181a_st | 1.866 | 0.04014 | 2.64 | 0.05783 |
| ptr-let-7d_st | 1.199 | 0.31854 | 2.48 | 0.05811 |
| fru-let-7b_st | 1.613 | 0.02224 | 2.19 | 0.05855 |
| SNORA38B_x_st | 1.484 | 0.14067 | 3.16 | 0.05860 |
| eca-miR-125b-5p_st | 15.263 | 0.02222 | 78.94 | 0.05903 |
| ssc-miR-222_st | 1.986 | 0.06750 | 2.95 | 0.05911 |
| ptr-miR-17-5p_st | 4.713 | 0.00168 | 5.41 | 0.05920 |
| xtr-miR-130a_st | 71.755 | 0.01083 | 36.83 | 0.05934 |
| dwi-miR-125_st | 16.899 | 0.00654 | 244.11 | 0.05947 |
| oan-miR-1357_st | 1.475 | 0.15335 | 7.44 | 0.05980 |
| mmu-miR-20a_st | 4.442 | 0.01300 | 7.84 | 0.05996 |
| U55_st | 0.876 | 0.37229 | 8.28 | 0.05997 |
| snR39B_x_st | 0.959 | 0.59998 | 6.09 | 0.06004 |
| mmu-miR-1839-3p_st | 1.647 | 0.01970 | 18.20 | 0.06048 |
| sla-miR-20_st | 4.748 | 0.02370 | 8.21 | 0.06061 |
| snR38C_st | 1.225 | 0.35697 | 4.72 | 0.06086 |
| ppy-miR-361-5p_st | 0.949 | 0.24847 | 2.94 | 0.06096 |
| U17b_st | 2.273 | 0.16676 | 3.27 | 0.06114 |
| dre-miR-126_st | 58.815 | 0.00325 | 42.37 | 0.06124 |
| lla-miR-20_st | 4.033 | 0.02023 | 7.85 | 0.06124 |
| cfa-let-7c_st | 1.285 | 0.24720 | 2.51 | 0.06134 |
| odi-let-7c_st | 1.530 | 0.30166 | 3.00 | 0.06137 |
| mdo-miR-20_st | 4.145 | 0.00970 | 5.81 | 0.06140 |
| eca-let-7e_st | 0.902 | 0.73644 | 4.47 | 0.06145 |
| dmo-miR-100_st | 4.214 | 0.32083 | 2.61 | 0.06148 |
| ptr-miR-20b_st | 6.607 | 0.03971 | 7.85 | 0.06158 |
| cte-miR-92b_st | 4.563 | 0.01973 | 4.69 | 0.06169 |
| ssc-let-7f_st | 1.152 | 0.26257 | 3.21 | 0.06197 |
| rno-miR-352_st | 1.075 | 0.73990 | 3.71 | 0.06205 |
| U107_st | 1.032 | 0.87099 | 13.28 | 0.06219 |
| mgh28S-2409_st | 0.993 | 0.94555 | 9.64 | 0.06226 |
| ppy-let-7e_st | 1.017 | 0.97096 | 3.26 | 0.06238 |
| rno-let-7c_st | 1.388 | 0.03540 | 2.83 | 0.06252 |
| U3-4_s_st | 2.087 | 0.27742 | 8.06 | 0.06269 |
| rno-miR-126_st | 74.410 | 0.00381 | 39.93 | 0.06269 |
| U57_st | 0.929 | 0.73037 | 15.97 | 0.06300 |
| mne-miR-99a_st | 21.051 | 0.00290 | 78.77 | 0.06302 |
| eca-miR-99b_st | 0.358 | 0.35577 | 5.91 | 0.06330 |
| ACA61_st | 0.916 | 0.87608 | 3.76 | 0.06339 |
| sko-let-7_st | 1.220 | 0.14594 | 2.94 | 0.06367 |
| ppa-miR-181c_st | 2.497 | 0.26929 | 4.75 | 0.06436 |
| lla-miR-17-5p_st | 5.313 | 0.01255 | 6.32 | 0.06438 |
| E3_x_st | 1.155 | 0.58004 | 3.13 | 0.06439 |
| gga-miR-155_st | 1.515 | 0.28965 | 3.57 | 0.06472 |
| ENSG00000202252_st | 0.563 | 0.05624 | 2.62 | 0.06476 |
| tgu-let-7a_st | 1.135 | 0.29373 | 2.79 | 0.06477 |
| mdo-let-7a_st | 1.208 | 0.10826 | 2.33 | 0.06490 |
| gga-miR-20a_st | 5.586 | 0.00521 | 7.08 | 0.06500 |
| tca-miR-125_st | 13.565 | 0.00267 | 170.40 | 0.06517 |
| ggo-miR-106a_st | 4.506 | 0.00497 | 4.63 | 0.06524 |
| mdo-miR-17-5p_st | 4.738 | 0.00592 | 5.51 | 0.06533 |
| crm-let-7_st | 1.144 | 0.06035 | 3.29 | 0.06546 |
| U3-2B_s_st | 1.917 | 0.22022 | 13.65 | 0.06563 |
| U71b_x_st | 1.834 | 0.14599 | 5.86 | 0.06571 |
| HBII-180C_x_st | 1.394 | 0.14794 | 97.57 | 0.06578 |
| bta-let-7d_st | 1.197 | 0.34182 | 2.78 | 0.06590 |
| ENSG00000200288_x_st | 1.309 | 0.08328 | 6.05 | 0.06595 |
| aga-miR-125_st | 24.938 | 0.01208 | 150.86 | 0.06599 |
| eca-miR-20a_st | 4.655 | 0.01386 | 6.69 | 0.06601 |
| fru-miR-17_st | 4.808 | 0.00025 | 7.61 | 0.06603 |
| dre-miR-126b_st | 107.301 | 0.00958 | 57.84 | 0.06637 |
| fru-miR-30b_st | 1.799 | 0.15606 | 3.25 | 0.06657 |
| HBII-202_st | 0.925 | 0.57962 | 5.59 | 0.06685 |
| dan-let-7_st | 1.157 | 0.20716 | 2.62 | 0.06688 |
| lca-miR-17-5p_st | 3.919 | 0.00594 | 6.15 | 0.06724 |
| ACA42_st | 1.774 | 0.28581 | 3.50 | 0.06739 |
| sla-miR-125b_st | 17.345 | 0.01447 | 170.54 | 0.06746 |
| cfa-miR-130a_st | 31.492 | 0.00035 | 24.70 | 0.06765 |
| ACA17_st | 2.574 | 0.10119 | 33.39 | 0.06773 |
| tgu-miR-20b_st | 7.096 | 0.01521 | 6.34 | 0.06803 |
| snR39B_s_st | 0.785 | 0.13035 | 6.51 | 0.06840 |
| fru-miR-130_st | 138.404 | 0.00289 | 86.43 | 0.06850 |
| eca-miR-106a_st | 3.991 | 0.00377 | 6.17 | 0.06881 |
| eca-miR-130a_st | 59.044 | 0.00524 | 51.62 | 0.06883 |
| bta-miR-20a_st | 5.498 | 0.00767 | 5.97 | 0.06884 |
| mml-miR-128a_st | 1.752 | 0.06301 | 4.18 | 0.06917 |
| tgu-miR-125-1-star_st | 1.614 | 0.30277 | 3.34 | 0.06938 |
| bfl-let-7_st | 1.253 | 0.05196 | 2.72 | 0.06939 |
| cin-let-7c_st | 1.192 | 0.51789 | 2.68 | 0.06939 |
| aae-miR-125_st | 21.593 | 0.00082 | 139.89 | 0.06947 |
| SNORD121B_st | 1.362 | 0.40378 | 3.23 | 0.06976 |
| ptr-miR-486_st | 21.089 | 0.11093 | 26.22 | 0.07001 |
| cfa-miR-20b_st | 7.305 | 0.00143 | 7.22 | 0.07007 |
| hsa-miR-320d_st | 1.725 | 0.08109 | 2.91 | 0.07061 |
| tni-miR-222_st | 1.945 | 0.02565 | 3.94 | 0.07078 |
| oan-miR-130c_st | 27.854 | 0.02445 | 45.45 | 0.07083 |
| hsa-let-7a_st | 1.213 | 0.13715 | 2.37 | 0.07084 |
| U73a_st | 0.885 | 0.45381 | 8.24 | 0.07085 |
| eca-miR-19b_st | 2.754 | 0.00804 | 4.61 | 0.07085 |
| HBII-85-4_x_st | 1.592 | 0.21184 | 3.49 | 0.07086 |
| gga-miR-181a_st | 2.154 | 0.03290 | 2.40 | 0.07090 |
| oan-miR-106_st | 4.770 | 0.00170 | 6.48 | 0.07094 |
| U27_st | 0.744 | 0.12952 | 11.95 | 0.07118 |
| tgu-let-7c_st | 1.153 | 0.39530 | 2.54 | 0.07167 |
| hsa-miR-221_st | 1.682 | 0.01649 | 2.84 | 0.07177 |
| mml-miR-30a-5p_st | 0.945 | 0.90018 | 2.45 | 0.07184 |
| bta-miR-181d_st | 4.318 | 0.05482 | 4.35 | 0.07213 |
| mml-miR-181d_st | 8.406 | 0.04743 | 6.19 | 0.07215 |
| hsa-miR-551a_st | 4.572 | 0.13278 | 2.10 | 0.07229 |
| mml-miR-363_st | 4.693 | 0.01956 | 4.76 | 0.07246 |
| mdo-miR-19b_st | 2.725 | 0.00943 | 4.81 | 0.07259 |
| xtr-miR-221_st | 2.057 | 0.03837 | 3.79 | 0.07262 |
| tni-miR-17_st | 5.999 | 0.00109 | 7.80 | 0.07281 |
| rno-miR-222_st | 2.280 | 0.04448 | 3.26 | 0.07286 |
| ptr-miR-374b_st | 2.237 | 0.38826 | 5.63 | 0.07313 |
| mmu-miR-106a_st | 3.467 | 0.00290 | 6.48 | 0.07314 |
| ssc-miR-125b_st | 17.351 | 0.00345 | 95.00 | 0.07320 |
| SNORD119_st | 0.808 | 0.48763 | 3.88 | 0.07323 |
| hsa-miR-126_st | 87.559 | 0.01065 | 49.53 | 0.07374 |
| hsa-let-7d_st | 1.241 | 0.41867 | 2.58 | 0.07374 |
| bta-miR-221_st | 2.061 | 0.00267 | 3.77 | 0.07402 |
| ppy-miR-196b_st | 36.923 | 0.03940 | 18.35 | 0.07425 |
| ame-miR-92b_st | 4.776 | 0.02692 | 13.91 | 0.07447 |
| v11_rno-miR-17_st | 4.585 | 0.00395 | 5.56 | 0.07455 |
| tgu-miR-130c_st | 38.484 | 0.01038 | 63.54 | 0.07458 |
| U59B_st | 0.713 | 0.02028 | 5.05 | 0.07461 |
| ptr-miR-126_st | 74.871 | 0.00482 | 50.39 | 0.07470 |
| rno-miR-17-5p_st | 4.146 | 0.01037 | 5.60 | 0.07513 |
| sla-miR-17-5p_st | 4.277 | 0.01083 | 8.82 | 0.07545 |
| U30_x_st | 0.941 | 0.52594 | 5.33 | 0.07556 |
| bta-miR-361_st | 1.249 | 0.22446 | 3.09 | 0.07556 |
| bta-miR-20b_st | 4.299 | 0.00751 | 6.46 | 0.07597 |
| eca-miR-221_st | 1.873 | 0.02550 | 3.05 | 0.07599 |
| isc-let-7_st | 1.179 | 0.23343 | 2.54 | 0.07610 |
| HBII-436_st | 0.291 | 0.06181 | 2.70 | 0.07612 |
| tni-miR-126_st | 53.753 | 0.01201 | 37.51 | 0.07640 |
| ppy-miR-19b_st | 2.965 | 0.01449 | 6.42 | 0.07678 |
| HBII-85-2_x_st | 1.527 | 0.30209 | 6.03 | 0.07684 |
| eca-miR-30b_st | 1.153 | 0.38478 | 3.22 | 0.07702 |
| lla-miR-93_st | 1.984 | 0.07515 | 3.15 | 0.07726 |
| ptr-miR-1271_st | 2.450 | 0.20100 | 3.14 | 0.07745 |
| mmu-miR-30b_st | 1.130 | 0.45332 | 3.32 | 0.07749 |
| U58A_st | 1.406 | 0.33492 | 5.65 | 0.07753 |
| cbr-let-7_st | 1.349 | 0.20150 | 3.22 | 0.07772 |
| mml-let-7a_st | 1.317 | 0.05865 | 2.63 | 0.07816 |
| U97_st | 1.286 | 0.57088 | 6.86 | 0.07819 |
| U38B_st | 0.828 | 0.32794 | 8.47 | 0.07822 |
| ACA15_x_st | 2.520 | 0.00701 | 7.69 | 0.07829 |
| U35B_st | 1.206 | 0.06776 | 2.83 | 0.07835 |
| mml-let-7c_st | 1.368 | 0.07602 | 2.52 | 0.07851 |
| hp_hsa-mir-1259_s_st | 5.074 | 0.12224 | 2.77 | 0.07855 |
| tgu-miR-126_st | 49.419 | 0.00935 | 39.01 | 0.07855 |
| mml-miR-130a_st | 50.052 | 0.01207 | 30.15 | 0.07858 |
| ppc-let-7_st | 1.209 | 0.08969 | 2.53 | 0.07870 |
| U79_st | 1.116 | 0.61105 | 7.64 | 0.07874 |
| ACA24_s_st | 2.040 | 0.00213 | 47.41 | 0.07892 |
| oan-miR-130b_st | 53.215 | 0.00178 | 40.32 | 0.07896 |
| hsa-let-7f_st | 1.352 | 0.12339 | 2.97 | 0.07925 |
| gga-miR-125b_st | 13.977 | 0.00186 | 233.04 | 0.07930 |
| hp_rno-mir-126_st | 8.416 | 0.21903 | 4.73 | 0.07930 |
| ppy-miR-221_st | 1.804 | 0.02524 | 3.71 | 0.07930 |
| U96a_x_st | 0.819 | 0.55387 | 6.56 | 0.07951 |
| fru-let-7d_st | 3.161 | 0.05603 | 6.46 | 0.07962 |
| rno-let-7d_st | 1.226 | 0.21458 | 2.57 | 0.07966 |
| oan-miR-126_st | 52.673 | 0.00608 | 36.94 | 0.07967 |
| dwi-let-7_st | 1.162 | 0.12523 | 2.56 | 0.07982 |
| fru-miR-30d_st | 1.117 | 0.21240 | 2.60 | 0.07990 |
| HBII-234_x_st | 1.481 | 0.08280 | 3.43 | 0.08014 |
| mdo-miR-222a_st | 1.943 | 0.09064 | 3.41 | 0.08019 |
| xtr-miR-320_st | 2.067 | 0.13300 | 2.30 | 0.08032 |
| U56_st | 0.969 | 0.86299 | 16.39 | 0.08079 |
| age-miR-106a_st | 4.857 | 0.01079 | 6.04 | 0.08115 |
| bmo-let-7_st | 1.296 | 0.08673 | 2.72 | 0.08131 |
| xtr-let-7b_st | 1.163 | 0.66184 | 3.76 | 0.08134 |
| rno-let-7e_st | 1.022 | 0.96828 | 3.26 | 0.08136 |
| ppy-miR-181c_st | 2.449 | 0.24187 | 4.22 | 0.08146 |
| cfa-miR-222_st | 2.354 | 0.03241 | 3.31 | 0.08147 |
| U3-3_s_st | 2.728 | 0.21209 | 14.77 | 0.08156 |
| eca-miR-490-5p_st | 2.474 | 0.01544 | 3.24 | 0.08192 |
| dre-miR-222_st | 2.134 | 0.06343 | 3.16 | 0.08217 |
| tni-let-7d_st | 3.067 | 0.03128 | 10.70 | 0.08252 |
| U41_st | 1.804 | 0.29129 | 8.19 | 0.08351 |
| ggo-miR-17-5p_st | 5.365 | 0.00829 | 6.11 | 0.08352 |
| ssc-miR-221_st | 1.590 | 0.02462 | 3.77 | 0.08355 |
| gga-miR-130c_st | 30.262 | 0.01172 | 30.81 | 0.08359 |
| lgi-let-7_st | 1.261 | 0.03642 | 2.49 | 0.08362 |
| cre-miR919.1_st | 1.002 | 0.99248 | 2.15 | 0.08395 |
| U83B_st | 0.765 | 0.19479 | 8.79 | 0.08417 |
| hsa-miR-20a_st | 5.284 | 0.01236 | 5.49 | 0.08426 |
| eca-let-7f_st | 1.418 | 0.12553 | 2.83 | 0.08431 |
| ssc-miR-106a_st | 5.219 | 0.00651 | 6.15 | 0.08440 |
| cfa-miR-125b_st | 12.618 | 0.00247 | 137.81 | 0.08449 |
| hsa-miR-664-star_st | 2.487 | 0.39109 | 3.14 | 0.08480 |
| bta-miR-126_st | 41.633 | 0.00866 | 51.49 | 0.08487 |
| U16_st | 1.351 | 0.36372 | 6.14 | 0.08506 |
| ptr-miR-93_st | 1.756 | 0.04226 | 3.05 | 0.08522 |
| U44_x_st | 0.907 | 0.07031 | 13.22 | 0.08538 |
| hsa-miR-181c_st | 2.798 | 0.19841 | 5.88 | 0.08560 |
| dre-miR-155_st | 0.955 | 0.88940 | 3.01 | 0.08561 |
| cin-miR-126_st | 101.452 | 0.01037 | 67.37 | 0.08569 |
| mml-miR-20a_st | 4.155 | 0.01864 | 6.31 | 0.08598 |
| oan-let-7e_st | 1.680 | 0.38133 | 3.45 | 0.08599 |
| lla-miR-99a_st | 15.925 | 0.00747 | 92.99 | 0.08606 |
| nvi-miR-125_st | 19.186 | 0.00456 | 212.18 | 0.08609 |
| rno-miR-181c_st | 2.950 | 0.12882 | 6.23 | 0.08617 |
| spu-miR-125_st | 21.639 | 0.00193 | 172.38 | 0.08631 |
| U42B_x_st | 0.785 | 0.50376 | 3.91 | 0.08638 |
| oan-miR-155_st | 1.063 | 0.73327 | 4.63 | 0.08640 |
| eca-miR-486-5p_st | 10.128 | 0.10022 | 8.94 | 0.08644 |
| rlcv-miR-rL1-4-3p_st | 1.890 | 0.04617 | 2.14 | 0.08654 |
| rno-miR-20b-5p_st | 9.293 | 0.04084 | 5.39 | 0.08681 |
| dmo-let-7_st | 1.181 | 0.01860 | 2.32 | 0.08681 |
| cte-let-7_st | 1.095 | 0.35111 | 3.07 | 0.08702 |
| ENSG00000206903_s_st | 3.436 | 0.00034 | 53.70 | 0.08704 |
| tgu-miR-106_st | 4.836 | 0.00435 | 5.09 | 0.08710 |
| ssc-miR-361-5p_st | 1.181 | 0.17652 | 3.71 | 0.08756 |
| ppa-miR-17-5p_st | 5.085 | 0.01214 | 5.36 | 0.08782 |
| dme-miR-1001_st | 1.271 | 0.34843 | 2.18 | 0.08789 |
| ptr-miR-106a_st | 4.794 | 0.00452 | 6.10 | 0.08862 |
| HBII-85-21_x_st | 1.116 | 0.77763 | 2.40 | 0.08875 |
| ppy-miR-20_st | 4.644 | 0.01481 | 7.92 | 0.08884 |
| ptr-miR-155_st | 1.391 | 0.04128 | 4.25 | 0.08886 |
| dre-let-7a_st | 1.441 | 0.14916 | 2.31 | 0.08915 |
| xtr-miR-126_st | 42.730 | 0.00061 | 68.92 | 0.08932 |
| mml-miR-146a_st | 0.997 | 0.99486 | 3.45 | 0.08960 |
| ENSG00000207062_s_st | 3.048 | 0.14477 | 5.85 | 0.08973 |
| xtr-miR-130c_st | 41.915 | 0.00125 | 55.10 | 0.09004 |
| cfa-miR-221_st | 1.855 | 0.00528 | 3.75 | 0.09013 |
| tgu-miR-222_st | 1.757 | 0.08336 | 2.57 | 0.09022 |
| mmu-miR-221_st | 1.630 | 0.11300 | 3.64 | 0.09031 |
| U72_x_st | 1.402 | 0.57828 | 5.33 | 0.09031 |
| age-miR-222_st | 1.826 | 0.13251 | 2.92 | 0.09035 |
| ppy-miR-374a_st | 1.626 | 0.50877 | 2.19 | 0.09036 |
| xtr-miR-30b_st | 0.896 | 0.33559 | 3.57 | 0.09037 |
| HBI-61_s_st | 1.242 | 0.62854 | 4.02 | 0.09048 |
| gga-miR-126_st | 117.205 | 0.00352 | 36.29 | 0.09052 |
| cfa-miR-19b_st | 2.792 | 0.01143 | 5.98 | 0.09057 |
| ptr-let-7a_st | 1.350 | 0.07648 | 2.77 | 0.09085 |
| ACA43_st | 3.758 | 0.25446 | 7.88 | 0.09108 |
| ACA54_st | 0.509 | 0.13712 | 6.56 | 0.09127 |
| mmu-miR-126-3p_st | 66.446 | 0.00083 | 41.22 | 0.09132 |
| ACA6_st | 1.551 | 0.30925 | 12.64 | 0.09142 |
| xtr-miR-99_st | 17.928 | 0.00898 | 106.73 | 0.09147 |
| age-miR-17-5p_st | 5.156 | 0.00256 | 6.31 | 0.09174 |
| fru-miR-126_st | 46.057 | 0.01363 | 43.18 | 0.09178 |
| cfa-miR-30b_st | 1.099 | 0.70143 | 3.40 | 0.09206 |
| mmu-miR-130a_st | 31.400 | 0.00156 | 49.44 | 0.09228 |
| bta-miR-100_st | 31.511 | 0.06511 | 47.30 | 0.09230 |
| U49B_x_st | 1.828 | 0.08536 | 2.98 | 0.09232 |
| csa-let-7c_st | 1.153 | 0.36408 | 2.09 | 0.09265 |
| bta-let-7e_st | 0.676 | 0.44173 | 3.93 | 0.09275 |
| U82_st | 1.258 | 0.11865 | 16.79 | 0.09282 |
| ENSG00000207130_s_st | 1.741 | 0.01966 | 38.24 | 0.09309 |
| ptr-miR-181c_st | 3.803 | 0.12010 | 5.35 | 0.09312 |
| HBII-142_st | 0.628 | 0.15891 | 7.89 | 0.09356 |
| mmu-miR-196b_st | 40.365 | 0.07178 | 21.64 | 0.09374 |
| tgu-let-7b_st | 1.664 | 0.05765 | 2.57 | 0.09401 |
| lca-miR-20_st | 4.074 | 0.00895 | 5.61 | 0.09434 |
| mml-miR-126_st | 67.097 | 0.00825 | 25.18 | 0.09437 |
| HBII-429_st | 0.846 | 0.48641 | 4.79 | 0.09442 |
| dmo-miR-125_st | 18.662 | 0.00840 | 102.72 | 0.09449 |
| ACA28_st | 1.522 | 0.28595 | 8.07 | 0.09462 |
| HBII-85-14_x_st | 1.583 | 0.41692 | 3.38 | 0.09468 |
| rno-miR-20a_st | 5.340 | 0.00909 | 5.97 | 0.09495 |
| rno-let-7a_st | 1.245 | 0.25442 | 2.39 | 0.09495 |
| oan-miR-19b_st | 2.745 | 0.00032 | 5.20 | 0.09510 |
| U24_st | 1.097 | 0.13790 | 4.16 | 0.09516 |
| ssc-miR-335_st | 11.154 | 0.05081 | 18.92 | 0.09542 |
| oan-miR-181c_st | 4.008 | 0.04585 | 4.43 | 0.09554 |
| HBII-135_x_st | 1.310 | 0.36480 | 9.50 | 0.09576 |
| csa-miR-126_st | 81.124 | 0.02720 | 48.65 | 0.09580 |
| cel-let-7_st | 1.165 | 0.33132 | 2.55 | 0.09595 |
| gga-miR-222_st | 1.964 | 0.04312 | 3.20 | 0.09605 |
| fru-let-7g_st | 1.022 | 0.87271 | 3.79 | 0.09607 |
| U18A_x_st | 0.848 | 0.51466 | 3.06 | 0.09615 |
| mmu-miR-181a-1-star_st | 2.965 | 0.08999 | 4.18 | 0.09626 |
| ENSG00000207410_x_st | 3.063 | 0.07015 | 3.31 | 0.09639 |
| bta-let-7f_st | 1.225 | 0.18932 | 2.78 | 0.09664 |
| U3-2_s_st | 2.296 | 0.20656 | 9.68 | 0.09696 |
| dre-miR-99_st | 20.858 | 0.00685 | 99.08 | 0.09697 |
| eca-miR-363_st | 3.742 | 0.01473 | 4.05 | 0.09708 |
| fru-miR-100_st | 31.946 | 0.05416 | 33.54 | 0.09720 |
| tca-let-7_st | 1.193 | 0.08670 | 2.84 | 0.09765 |
| ssc-miR-196b_st | 73.895 | 0.00745 | 40.45 | 0.09766 |
| cfa-miR-99a_st | 11.803 | 0.00914 | 81.84 | 0.09767 |
| hp_rno-mir-17-1_x_st | 2.959 | 0.06139 | 3.73 | 0.09823 |
| cqu-miR-125_st | 10.887 | 0.02103 | 93.72 | 0.09830 |
| U78_x_st | 1.884 | 0.22532 | 4.67 | 0.09843 |
| hp_mmu-mir-126_st | 6.315 | 0.01691 | 3.08 | 0.09857 |
| bta-miR-181c_st | 3.504 | 0.04779 | 5.57 | 0.09885 |
| dre-miR-19d_st | 3.303 | 0.01734 | 5.86 | 0.09891 |
| ssc-miR-181c_st | 2.521 | 0.10228 | 3.07 | 0.09892 |
| dps-let-7_st | 1.276 | 0.05303 | 2.58 | 0.09892 |
| gga-miR-221_st | 1.681 | 0.02721 | 3.48 | 0.09896 |
| U62B_s_st | 1.122 | 0.75577 | 8.29 | 0.09905 |
| ptr-miR-130b_st | 4.526 | 0.03668 | 14.21 | 0.09966 |
| lla-miR-25_st | 1.775 | 0.05896 | 3.34 | 0.09967 |
| mdo-miR-181c_st | 2.029 | 0.10842 | 2.07 | 0.09981 |
| cfa-miR-25_st | 1.708 | 0.00392 | 2.83 | 0.10026 |
| mgU6-77_st | 1.552 | 0.46382 | 94.13 | 0.10074 |
| eca-miR-99a_st | 22.843 | 0.01962 | 149.55 | 0.10077 |
| mdo-miR-146a_st | 0.878 | 0.81154 | 3.51 | 0.10103 |
| eca-miR-126-3p_st | 42.038 | 0.00066 | 41.34 | 0.10132 |
| ssc-let-7a_st | 1.265 | 0.30592 | 2.56 | 0.10141 |
| mmu-miR-423-3p_st | 1.100 | 0.69599 | 4.09 | 0.10166 |
| bta-miR-30c_st | 0.858 | 0.49218 | 2.54 | 0.10173 |
| ppy-miR-126_st | 53.796 | 0.00843 | 34.31 | 0.10176 |
| ENSG00000212615_x_st | 0.814 | 0.51327 | 4.46 | 0.10183 |
| cqu-miR-100_st | 20.842 | 0.02265 | 37.50 | 0.10191 |
| ACA33_st | 3.072 | 0.02115 | 8.77 | 0.10216 |
| ppa-miR-181b_st | 2.794 | 0.05781 | 3.21 | 0.10222 |
| ENSG00000221164_x_st | 1.349 | 0.62375 | 2.31 | 0.10270 |
| U35A_st | 0.778 | 0.54484 | 8.27 | 0.10281 |
| eca-miR-196b_st | 56.946 | 0.05138 | 26.17 | 0.10305 |
| mdo-miR-93_st | 1.862 | 0.04620 | 3.82 | 0.10308 |
| ENSG00000252840_s_st | 1.572 | 0.31965 | 16.33 | 0.10331 |
| cfa-miR-374b_st | 1.888 | 0.42871 | 2.84 | 0.10368 |
| sla-miR-19b_st | 2.931 | 0.00686 | 5.33 | 0.10453 |
| hsa-miR-222_st | 2.089 | 0.04507 | 3.23 | 0.10455 |
| hsa-let-7e_st | 1.322 | 0.62906 | 3.43 | 0.10486 |
| mml-miR-20b_st | 5.038 | 0.04441 | 7.62 | 0.10497 |
| ENSG00000201042_x_st | 1.058 | 0.73952 | 3.06 | 0.10540 |
| ssc-miR-130a_st | 39.858 | 0.00095 | 46.31 | 0.10610 |
| xla-miR-19b_st | 3.396 | 0.00188 | 4.33 | 0.10620 |
| lla-miR-19b_st | 3.915 | 0.00654 | 4.51 | 0.10621 |
| ACA34_st | 1.493 | 0.25740 | 11.25 | 0.10626 |
| ppy-miR-30d_st | 1.102 | 0.53795 | 2.14 | 0.10630 |
| mmu-miR-93_st | 1.910 | 0.12015 | 4.06 | 0.10637 |
| HBI-100_st | 1.176 | 0.10895 | 2.98 | 0.10645 |
| ACA58_x_st | 2.369 | 0.21154 | 5.61 | 0.10648 |
| tni-miR-221_st | 1.792 | 0.05718 | 3.85 | 0.10693 |
| oan-miR-20a_st | 9.742 | 0.04738 | 14.00 | 0.10697 |
| mml-miR-19b_st | 3.680 | 0.00704 | 4.54 | 0.10727 |
| rno-miR-322_st | 2.370 | 0.28247 | 3.56 | 0.10744 |
| dpu-miR-10_st | 118.855 | 0.01266 | 142.39 | 0.10746 |
| SNORD125_st | 0.924 | 0.67966 | 4.72 | 0.10779 |
| dre-let-7e_st | 1.211 | 0.36558 | 5.50 | 0.10789 |
| mne-miR-30c_st | 0.854 | 0.28609 | 2.33 | 0.10794 |
| fru-miR-25_st | 1.686 | 0.07555 | 2.57 | 0.10805 |
| ENSG00000252213_st | 7.723 | 0.02540 | 18.26 | 0.10828 |
| rno-miR-30b-5p_st | 1.628 | 0.13000 | 2.73 | 0.10926 |
| rno-miR-99a_st | 14.328 | 0.01276 | 142.85 | 0.10933 |
| ptr-miR-664_st | 3.086 | 0.09075 | 4.33 | 0.10939 |
| bta-miR-222_st | 1.766 | 0.11403 | 3.22 | 0.10958 |
| ame-let-7_st | 1.120 | 0.00507 | 2.56 | 0.10989 |
| cfa-miR-196b_st | 72.380 | 0.03099 | 47.02 | 0.11036 |
| ppy-miR-222_st | 1.847 | 0.10182 | 3.02 | 0.11041 |
| gga-miR-1564_st | 1.365 | 0.37514 | 2.83 | 0.11060 |
| tca-miR-100_st | 23.828 | 0.05200 | 51.06 | 0.11076 |
| ggo-miR-19b_st | 2.769 | 0.00315 | 4.43 | 0.11090 |
| U32B_x_st | 0.674 | 0.41686 | 2.08 | 0.11095 |
| ACA32_st | 1.327 | 0.02240 | 6.09 | 0.11187 |
| fru-miR-181a-star_st | 2.623 | 0.16187 | 4.23 | 0.11251 |
| mne-miR-20_st | 4.563 | 0.01910 | 8.55 | 0.11255 |
| ACA20_x_st | 0.930 | 0.91189 | 4.66 | 0.11260 |
| dre-let-7b_st | 1.992 | 0.00956 | 2.57 | 0.11291 |
| mml-miR-30c_st | 0.712 | 0.23579 | 2.29 | 0.11300 |
| ggo-miR-99a_st | 15.539 | 0.00369 | 110.63 | 0.11357 |
| ppy-let-7f_st | 1.275 | 0.03632 | 2.45 | 0.11367 |
| U49A_s_st | 1.276 | 0.52802 | 12.42 | 0.11419 |
| v11_hsa-miR-768-3p_st | 0.486 | 0.00604 | 7.32 | 0.11488 |
| ENSG00000199411_s_st | 0.782 | 0.41821 | 2.18 | 0.11498 |
| ACA21_st | 1.571 | 0.28596 | 3.10 | 0.11501 |
| ggo-miR-221_st | 2.070 | 0.03021 | 2.83 | 0.11525 |
| ppy-miR-99a_st | 21.642 | 0.03143 | 143.12 | 0.11549 |
| age-miR-10a_st | 140.744 | 0.04072 | 126.20 | 0.11569 |
| ACA3-2_st | 0.760 | 0.57316 | 26.31 | 0.11610 |
| U75_st | 1.268 | 0.64276 | 2.20 | 0.11614 |
| U52_st | 0.971 | 0.91927 | 9.39 | 0.11620 |
| gga-miR-99a_st | 13.435 | 0.01145 | 134.46 | 0.11621 |
| U65_st | 3.505 | 0.22210 | 6.52 | 0.11627 |
| oan-miR-146a_st | 0.707 | 0.53403 | 2.80 | 0.11634 |
| mml-miR-155_st | 1.156 | 0.55848 | 3.75 | 0.11644 |
| gga-miR-30b_st | 1.531 | 0.23444 | 2.85 | 0.11663 |
| tgu-miR-30d-5p_st | 1.076 | 0.62823 | 2.20 | 0.11693 |
| HBII-251_st | 0.975 | 0.94509 | 7.75 | 0.11711 |
| gga-miR-10a_st | 106.585 | 0.04404 | 175.39 | 0.11711 |
| U71c_st | 1.646 | 0.29547 | 4.19 | 0.11758 |
| gga-miR-19b_st | 3.924 | 0.01953 | 5.52 | 0.11780 |
| hsa-miR-93_st | 1.994 | 0.02067 | 3.79 | 0.11838 |
| ACA26_st | 2.003 | 0.14560 | 4.83 | 0.11878 |
| der-let-7_st | 1.114 | 0.28034 | 2.20 | 0.11883 |
| hsa-miR-30b_st | 1.421 | 0.24490 | 3.30 | 0.11889 |
| ssc-miR-19b_st | 2.823 | 0.00112 | 5.00 | 0.11891 |
| mml-miR-30b_st | 1.216 | 0.41861 | 3.13 | 0.11891 |
| ppy-miR-30b_st | 1.223 | 0.45114 | 3.07 | 0.11914 |
| oan-let-7f_st | 1.196 | 0.16058 | 2.91 | 0.11947 |
| U77_st | 1.776 | 0.13534 | 3.03 | 0.11954 |
| ppy-miR-20b_st | 5.892 | 0.01874 | 6.20 | 0.11958 |
| ACA41_st | 1.895 | 0.17257 | 5.04 | 0.12010 |
| fru-miR-19b_st | 2.693 | 0.00002 | 6.43 | 0.12019 |
| tgu-miR-146c_st | 0.891 | 0.80300 | 3.74 | 0.12021 |
| fru-miR-221_st | 1.538 | 0.09515 | 3.06 | 0.12028 |
| eca-miR-423-3p_st | 1.304 | 0.24679 | 5.33 | 0.12036 |
| cqu-let-7_st | 1.703 | 0.11974 | 3.21 | 0.12051 |
| bfl-miR-100_st | 29.969 | 0.03172 | 45.41 | 0.12069 |
| HBII-85-3_x_st | 1.375 | 0.46138 | 2.13 | 0.12071 |
| xtr-let-7f_st | 1.387 | 0.10862 | 2.36 | 0.12073 |
| ACA48_st | 1.409 | 0.31614 | 2.91 | 0.12103 |
| ame-miR-100_st | 21.271 | 0.03322 | 53.81 | 0.12103 |
| ENSG00000207118_x_st | 0.995 | 0.98106 | 3.05 | 0.12141 |
| mmu-miR-19b_st | 3.089 | 0.00652 | 5.49 | 0.12142 |
| HBII-95B_st | 0.925 | 0.80860 | 2.12 | 0.12162 |
| ptr-let-7e_st | 1.071 | 0.88827 | 2.64 | 0.12165 |
| ggo-miR-25_st | 1.537 | 0.02163 | 3.30 | 0.12187 |
| hp_mmu-mir-20b_st | 1.240 | 0.28002 | 2.22 | 0.12206 |
| ACA33_x_st | 2.144 | 0.11124 | 6.62 | 0.12220 |
| rno-miR-130b_st | 3.031 | 0.05305 | 7.52 | 0.12221 |
| eca-miR-222_st | 1.876 | 0.07683 | 3.45 | 0.12260 |
| U60_x_st | 0.777 | 0.47355 | 9.03 | 0.12298 |
| rno-miR-361_st | 1.114 | 0.25444 | 3.28 | 0.12301 |
| U77_x_st | 2.163 | 0.04014 | 2.49 | 0.12320 |
| gga-let-7f_st | 1.299 | 0.07405 | 2.58 | 0.12334 |
| mml-miR-99a_st | 18.591 | 0.00637 | 123.61 | 0.12358 |
| cqu-miR-10-star_st | 98.178 | 0.02590 | 168.91 | 0.12400 |
| HBII-296B_x_st | 0.940 | 0.74152 | 6.12 | 0.12422 |
| nvi-miR-10_st | 133.177 | 0.04273 | 120.04 | 0.12423 |
| ppa-miR-10a_st | 142.467 | 0.01060 | 188.10 | 0.12521 |
| mne-miR-181a_st | 2.226 | 0.03199 | 2.65 | 0.12553 |
| dre-miR-19c_st | 3.531 | 0.00246 | 5.02 | 0.12560 |
| rno-miR-30d_st | 0.885 | 0.36330 | 2.72 | 0.12563 |
| dpe-let-7_st | 1.170 | 0.21514 | 2.35 | 0.12566 |
| ppy-miR-146a_st | 0.855 | 0.71770 | 2.98 | 0.12633 |
| hsa-miR-146a_st | 0.932 | 0.89575 | 4.64 | 0.12641 |
| dre-miR-19b_st | 2.915 | 0.00764 | 4.56 | 0.12646 |
| ppa-miR-30d_st | 1.039 | 0.81821 | 2.32 | 0.12665 |
| U106_st | 1.268 | 0.00156 | 6.10 | 0.12666 |
| tgu-miR-130a_st | 69.174 | 0.00816 | 30.28 | 0.12667 |
| ENSG00000212378_s_st | 1.589 | 0.17399 | 5.79 | 0.12679 |
| U106_x_st | 1.396 | 0.12723 | 7.74 | 0.12684 |
| mne-miR-25_st | 1.589 | 0.00935 | 3.43 | 0.12701 |
| fru-miR-15b_st | 1.380 | 0.34943 | 2.51 | 0.12804 |
| ppa-miR-100_st | 22.032 | 0.02989 | 48.68 | 0.12814 |
| hp_mmu-mir-130a_st | 1.895 | 0.01586 | 2.81 | 0.12863 |
| lca-miR-19b_st | 3.137 | 0.00329 | 5.89 | 0.12864 |
| hp_rno-let-7b_x_st | 1.053 | 0.92538 | 2.44 | 0.12886 |
| ENSG00000201592_s_st | 2.467 | 0.05826 | 2.61 | 0.12895 |
| ppy-miR-93_st | 2.293 | 0.10633 | 4.13 | 0.12911 |
| ppa-miR-99a_st | 19.451 | 0.01274 | 85.49 | 0.12915 |
| hsa-miR-338-5p_st | 0.566 | 0.58063 | 2.85 | 0.12918 |
| oan-miR-181a-star_st | 4.397 | 0.03242 | 5.27 | 0.12954 |
| ppa-miR-20_st | 4.401 | 0.02575 | 6.07 | 0.12958 |
| ppy-miR-335_st | 15.075 | 0.08304 | 25.08 | 0.13008 |
| U102_st | 0.878 | 0.52674 | 8.91 | 0.13030 |
| tni-let-7g_st | 1.020 | 0.92840 | 3.34 | 0.13039 |
| ACA15_s_st | 2.934 | 0.11304 | 7.42 | 0.13095 |
| HBII-85-19_x_st | 1.608 | 0.25458 | 6.74 | 0.13109 |
| ENSG00000206603_s_st | 2.872 | 0.41981 | 26.40 | 0.13116 |
| U102_x_st | 0.997 | 0.99161 | 2.50 | 0.13141 |
| U15A_st | 3.314 | 0.17897 | 7.46 | 0.13155 |
| mmu-miR-100_st | 23.397 | 0.02565 | 42.17 | 0.13172 |
| ppy-miR-664_st | 2.531 | 0.32051 | 3.77 | 0.13196 |
| U22_st | 1.030 | 0.92951 | 10.18 | 0.13197 |
| ptr-miR-30b_st | 1.103 | 0.71322 | 2.80 | 0.13211 |
| csa-let-7a_st | 1.052 | 0.82239 | 3.61 | 0.13224 |
| ACA47_st | 2.160 | 0.31734 | 3.08 | 0.13228 |
| U95_x_st | 0.778 | 0.14707 | 5.28 | 0.13229 |
| ppa-miR-25_st | 1.541 | 0.02135 | 3.25 | 0.13251 |
| oan-miR-30b_st | 1.057 | 0.63916 | 3.10 | 0.13295 |
| ssc-miR-28-5p_st | 0.975 | 0.79816 | 2.03 | 0.13312 |
| lla-miR-30b_st | 1.479 | 0.11088 | 2.36 | 0.13320 |
| tni-miR-100_st | 32.425 | 0.00883 | 64.17 | 0.13330 |
| U66_st | 2.432 | 0.12318 | 10.45 | 0.13337 |
| eca-let-7a_st | 1.260 | 0.14659 | 2.60 | 0.13348 |
| age-miR-19b_st | 3.609 | 0.00794 | 5.47 | 0.13362 |
| ppy-miR-146b-5p_st | 0.957 | 0.80283 | 3.85 | 0.13383 |
| aga-miR-100_st | 30.142 | 0.02293 | 37.12 | 0.13386 |
| eca-miR-100_st | 20.284 | 0.02526 | 49.72 | 0.13399 |
| HBII-85-5_x_st | 1.938 | 0.20932 | 2.25 | 0.13404 |
| mmu-let-7f_st | 1.379 | 0.14141 | 2.67 | 0.13418 |
| bfl-miR-10b_st | 2.424 | 0.05334 | 2.56 | 0.13420 |
| eca-miR-25_st | 1.939 | 0.02517 | 2.82 | 0.13438 |
| dre-miR-128_st | 1.911 | 0.12077 | 4.30 | 0.13442 |
| hsa-miR-1271_st | 2.154 | 0.24391 | 3.75 | 0.13563 |
| gga-miR-17-3p_st | 5.620 | 0.05345 | 7.69 | 0.13568 |
| ggo-miR-100_st | 16.955 | 0.02327 | 49.83 | 0.13572 |
| oan-miR-99_st | 15.686 | 0.00778 | 142.89 | 0.13652 |
| ppy-miR-155_st | 1.198 | 0.54472 | 3.40 | 0.13661 |
| U36B_st | 1.471 | 0.03597 | 12.13 | 0.13694 |
| mmu-miR-146a_st | 0.899 | 0.77969 | 3.30 | 0.13708 |
| oan-miR-222a_st | 1.857 | 0.10377 | 3.06 | 0.13717 |
| rno-let-7f_st | 1.301 | 0.20344 | 2.42 | 0.13764 |
| mml-miR-30d_st | 0.961 | 0.83344 | 2.78 | 0.13773 |
| bta-miR-335_st | 8.878 | 0.04274 | 26.46 | 0.13778 |
| eca-miR-335_st | 13.213 | 0.03016 | 53.07 | 0.13783 |
| mmu-miR-10a_st | 133.683 | 0.02853 | 276.57 | 0.13790 |
| hsa-miR-10a_st | 122.877 | 0.02130 | 227.19 | 0.13845 |
| ACA4_st | 1.723 | 0.23794 | 2.42 | 0.13847 |
| bta-miR-19b_st | 2.770 | 0.00944 | 5.36 | 0.13896 |
| ppa-miR-19b_st | 3.271 | 0.01343 | 5.26 | 0.13929 |
| lla-miR-30c_st | 0.927 | 0.75661 | 2.02 | 0.13940 |
| rno-miR-196b_st | 62.078 | 0.02632 | 26.91 | 0.13947 |
| ACA16_x_st | 1.011 | 0.95853 | 18.87 | 0.13952 |
| ssc-miR-99a_st | 22.422 | 0.00591 | 94.99 | 0.13964 |
| tgu-let-7e_st | 1.166 | 0.65908 | 4.13 | 0.13971 |
| hsa-miR-155_st | 1.162 | 0.53223 | 3.26 | 0.13973 |
| ENSG00000238936_x_st | 0.890 | 0.71599 | 3.71 | 0.13993 |
| ptr-miR-335_st | 10.222 | 0.03904 | 17.78 | 0.14036 |
| nve-miR-100_st | 21.872 | 0.04761 | 60.17 | 0.14037 |
| ggo-miR-26a_st | 1.203 | 0.07198 | 2.20 | 0.14055 |
| SNORA84_st | 2.849 | 0.12171 | 3.28 | 0.14109 |
| rno-miR-221_st | 1.529 | 0.06872 | 2.85 | 0.14118 |
| hsa-miR-335_st | 14.667 | 0.06697 | 33.55 | 0.14124 |
| lgi-miR-100_st | 31.895 | 0.04602 | 39.97 | 0.14131 |
| rno-miR-19b_st | 3.196 | 0.00888 | 4.51 | 0.14140 |
| bma-miR-100b_st | 15.432 | 0.00367 | 68.56 | 0.14140 |
| U38A_x_st | 0.699 | 0.23868 | 4.83 | 0.14179 |
| bta-miR-99a_st | 12.357 | 0.02412 | 116.51 | 0.14184 |
| ACA3_st | 1.369 | 0.25739 | 5.19 | 0.14190 |
| ACA41_x_st | 1.877 | 0.15705 | 4.07 | 0.14216 |
| ACA2a_st | 2.534 | 0.12501 | 2.67 | 0.14220 |
| ptr-miR-221_st | 1.685 | 0.02846 | 3.00 | 0.14229 |
| gga-let-7k_st | 1.261 | 0.58093 | 3.94 | 0.14236 |
| bmo-miR-2733c_st | 0.976 | 0.94031 | 2.14 | 0.14246 |
| ggo-miR-30a-3p_st | 1.449 | 0.46356 | 2.03 | 0.14269 |
| mne-miR-30d_st | 1.202 | 0.04393 | 2.40 | 0.14272 |
| ggo-miR-30b_st | 1.165 | 0.33874 | 2.45 | 0.14280 |
| cfa-miR-335_st | 11.890 | 0.03790 | 40.59 | 0.14291 |
| sla-miR-93_st | 2.170 | 0.01484 | 2.79 | 0.14303 |
| hsa-miR-363_st | 4.903 | 0.02900 | 3.96 | 0.14330 |
| mml-miR-100_st | 19.431 | 0.01926 | 42.72 | 0.14348 |
| spu-miR-10_st | 158.609 | 0.02680 | 233.10 | 0.14455 |
| xtr-miR-25_st | 1.667 | 0.01082 | 3.07 | 0.14481 |
| mmu-miR-99a_st | 16.648 | 0.00856 | 113.59 | 0.14491 |
| v49_ENSG00000201863_st | 0.802 | 0.14959 | 2.14 | 0.14532 |
| sko-miR-10_st | 124.934 | 0.02256 | 160.89 | 0.14568 |
| dre-miR-19a_st | 3.122 | 0.01340 | 5.54 | 0.14569 |
| hsa-miR-10b_st | 34.371 | 0.19220 | 29.87 | 0.14586 |
| U74_x_st | 1.082 | 0.62075 | 3.90 | 0.14596 |
| hsa-miR-622_st | 0.774 | 0.61324 | 2.67 | 0.14600 |
| bta-miR-130b_st | 3.287 | 0.00694 | 5.47 | 0.14603 |
| HBII-95_st | 1.172 | 0.60227 | 2.78 | 0.14618 |
| sla-miR-100_st | 21.924 | 0.08885 | 51.44 | 0.14619 |
| gga-miR-146a_st | 0.842 | 0.76426 | 3.81 | 0.14631 |
| ppy-miR-26a_st | 1.151 | 0.19936 | 2.52 | 0.14651 |
| age-miR-100_st | 24.142 | 0.00554 | 102.31 | 0.14658 |
| ppy-miR-181d_st | 6.992 | 0.02377 | 4.54 | 0.14681 |
| cfa-let-7f_st | 1.249 | 0.14603 | 2.60 | 0.14686 |
| bta-miR-486_st | 34.782 | 0.03125 | 7.27 | 0.14702 |
| U83A_st | 3.017 | 0.16706 | 4.31 | 0.14734 |
| mml-miR-19a_st | 3.591 | 0.03371 | 5.53 | 0.14783 |
| dsi-miR-10_st | 142.249 | 0.05320 | 85.75 | 0.14846 |
| mml-miR-181a_st | 1.868 | 0.09146 | 2.09 | 0.14962 |
| hsa-miR-19b_st | 3.378 | 0.00307 | 4.44 | 0.14968 |
| mne-miR-30b_st | 1.342 | 0.23838 | 2.35 | 0.14980 |
| mml-miR-221_st | 2.071 | 0.01352 | 3.16 | 0.14995 |
| mdo-let-7f_st | 1.255 | 0.21599 | 2.52 | 0.15010 |
| sko-miR-100_st | 24.436 | 0.01108 | 37.80 | 0.15043 |
| hsa-miR-93-star_st | 4.195 | 0.08571 | 14.20 | 0.15063 |
| U49A_st | 1.133 | 0.40371 | 7.93 | 0.15078 |
| mmu-miR-361_st | 1.468 | 0.07273 | 3.59 | 0.15082 |
| mml-miR-93_st | 2.563 | 0.02542 | 2.32 | 0.15083 |
| dwi-miR-100_st | 3.506 | 0.25606 | 3.13 | 0.15129 |
| U32A_x_st | 0.686 | 0.10304 | 3.45 | 0.15141 |
| bfl-miR-10a_st | 125.161 | 0.04017 | 188.10 | 0.15150 |
| eca-miR-10a_st | 180.119 | 0.03300 | 147.08 | 0.15169 |
| sla-miR-10a_st | 142.387 | 0.02703 | 190.20 | 0.15175 |
| cfa-miR-10_st | 64.001 | 0.03783 | 226.02 | 0.15179 |
| api-miR-10_st | 102.927 | 0.05601 | 102.07 | 0.15191 |
| mdo-miR-100_st | 19.263 | 0.07830 | 41.86 | 0.15193 |
| dre-miR-100_st | 20.851 | 0.00127 | 55.30 | 0.15211 |
| hsa-miR-99a_st | 21.563 | 0.00232 | 130.73 | 0.15247 |
| mdo-miR-19a_st | 2.542 | 0.00895 | 7.54 | 0.15271 |
| U46_x_st | 1.160 | 0.66657 | 5.21 | 0.15280 |
| isc-miR-100_st | 27.629 | 0.04461 | 43.51 | 0.15310 |
| ACA67_x_st | 1.499 | 0.18622 | 7.84 | 0.15313 |
| U21_st | 0.923 | 0.83375 | 4.02 | 0.15320 |
| dps-miR-10_st | 148.054 | 0.04296 | 156.45 | 0.15349 |
| der-miR-10_st | 114.335 | 0.06964 | 105.08 | 0.15411 |
| U68_st | 1.884 | 0.12963 | 9.28 | 0.15432 |
| U49B_s_st | 1.089 | 0.81938 | 5.75 | 0.15436 |
| mml-miR-222_st | 1.892 | 0.05854 | 3.12 | 0.15440 |
| lmi-miR-10_st | 88.982 | 0.05895 | 161.31 | 0.15441 |
| rno-miR-146a_st | 0.970 | 0.95898 | 3.03 | 0.15449 |
| tgu-miR-26_st | 1.171 | 0.08284 | 2.37 | 0.15483 |
| tgu-miR-99_st | 14.924 | 0.00011 | 76.69 | 0.15517 |
| mml-miR-10a_st | 90.953 | 0.03331 | 158.99 | 0.15564 |
| lca-miR-19a_st | 2.499 | 0.05043 | 5.97 | 0.15570 |
| U78_s_st | 1.830 | 0.14741 | 7.31 | 0.15608 |
| rno-miR-93_st | 2.112 | 0.09034 | 2.64 | 0.15631 |
| tni-miR-30b_st | 1.072 | 0.68156 | 2.92 | 0.15631 |
| ACA9_x_st | 2.554 | 0.01968 | 13.25 | 0.15659 |
| aae-miR-100_st | 33.500 | 0.02562 | 56.39 | 0.15659 |
| U47_st | 1.093 | 0.63360 | 8.79 | 0.15736 |
| mmu-miR-335-5p_st | 5.791 | 0.02869 | 31.09 | 0.15787 |
| mmu-miR-125a-5p_st | 0.666 | 0.53724 | 2.57 | 0.15824 |
| dps-miR-2507b-star_st | 1.460 | 0.28696 | 2.48 | 0.15839 |
| ggo-miR-93_st | 2.029 | 0.06887 | 2.89 | 0.15841 |
| cfa-miR-361_st | 1.297 | 0.22045 | 2.79 | 0.15843 |
| HBII-382_s_st | 0.782 | 0.01287 | 4.37 | 0.15902 |
| mdo-miR-25_st | 1.544 | 0.05813 | 2.63 | 0.15907 |
| mmu-miR-1949_st | 1.871 | 0.01206 | 9.65 | 0.15926 |
| hp_hsa-mir-26a-1_x_st | 0.785 | 0.39937 | 2.51 | 0.15938 |
| tgu-miR-181a-star_st | 5.035 | 0.07495 | 4.65 | 0.15966 |
| bta-miR-26a_st | 1.200 | 0.15681 | 2.29 | 0.15966 |
| mml-miR-551a_st | 3.207 | 0.16737 | 2.55 | 0.16005 |
| mdo-miR-221_st | 1.842 | 0.00651 | 3.47 | 0.16028 |
| ppy-miR-130b_st | 3.459 | 0.01610 | 3.62 | 0.16033 |
| cin-miR-155_st | 1.197 | 0.54289 | 2.14 | 0.16128 |
| bta-miR-128_st | 1.912 | 0.20621 | 2.49 | 0.16140 |
| gga-miR-128_st | 1.787 | 0.08113 | 3.00 | 0.16155 |
| tgu-miR-19b_st | 2.758 | 0.00611 | 5.51 | 0.16155 |
| U23_x_st | 2.084 | 0.04886 | 3.68 | 0.16194 |
| hsa-miR-374b_st | 2.861 | 0.31242 | 6.09 | 0.16230 |
| xtr-miR-19b_st | 2.819 | 0.01196 | 5.22 | 0.16246 |
| ENSG00000239145_x_st | 1.814 | 0.21620 | 15.06 | 0.16261 |
| ppa-miR-221_st | 1.442 | 0.06295 | 2.62 | 0.16294 |
| lgi-miR-10_st | 91.110 | 0.05369 | 121.09 | 0.16306 |
| eca-miR-361-5p_st | 0.946 | 0.69979 | 3.05 | 0.16367 |
| mmu-miR-181a-2-star_st | 5.058 | 0.03004 | 2.89 | 0.16369 |
| mmu-miR-130b_st | 3.875 | 0.06627 | 5.36 | 0.16370 |
| U43_x_st | 0.638 | 0.04769 | 8.37 | 0.16396 |
| fru-miR-26_st | 1.132 | 0.17122 | 2.35 | 0.16421 |
| oan-let-7d_st | 1.043 | 0.78625 | 2.42 | 0.16457 |
| mml-miR-25_st | 1.685 | 0.01051 | 3.18 | 0.16469 |
| ptr-miR-99a_st | 26.376 | 0.00924 | 123.07 | 0.16491 |
| sla-miR-17-3p_st | 5.372 | 0.00029 | 13.21 | 0.16494 |
| dpu-miR-100_st | 17.103 | 0.06187 | 63.79 | 0.16533 |
| tgu-miR-221_st | 1.731 | 0.05570 | 3.52 | 0.16535 |
| ame-miR-10_st | 122.100 | 0.02149 | 122.50 | 0.16553 |
| hsa-miR-196b_st | 47.441 | 0.01903 | 17.02 | 0.16554 |
| bmo-miR-100_st | 30.247 | 0.03166 | 52.89 | 0.16589 |
| U58C_st | 1.373 | 0.07517 | 6.17 | 0.16646 |
| mmu-miR-10b_st | 18.454 | 0.06319 | 20.51 | 0.16707 |
| dre-miR-126b-star_st | 80.628 | 0.00662 | 45.74 | 0.16716 |
| xtr-miR-18a-star_st | 3.655 | 0.02824 | 3.68 | 0.16725 |
| ACA63_st | 2.022 | 0.16930 | 6.05 | 0.16738 |
| mml-miR-146b-5p_st | 0.713 | 0.19309 | 3.67 | 0.16749 |
| ACA7B_s_st | 1.133 | 0.68691 | 4.03 | 0.16766 |
| ptr-miR-181a-star_st | 5.773 | 0.04831 | 2.81 | 0.16793 |
| tca-miR-10_st | 83.217 | 0.04117 | 140.86 | 0.16802 |
| ssc-miR-30b-5p_st | 1.321 | 0.24574 | 2.50 | 0.16813 |
| ACA32_x_st | 1.400 | 0.21452 | 6.80 | 0.16877 |
| mml-miR-335_st | 15.637 | 0.06458 | 23.55 | 0.16880 |
| ptr-miR-10a_st | 106.355 | 0.04117 | 232.18 | 0.16900 |
| U60_st | 0.968 | 0.93299 | 8.74 | 0.16905 |
| cfa-miR-146a_st | 0.906 | 0.84930 | 3.11 | 0.16919 |
| HBI-6_x_st | 1.567 | 0.05087 | 9.62 | 0.16930 |
| bta-miR-155_st | 1.171 | 0.43938 | 3.74 | 0.16932 |
| rno-miR-18a_st | 2.278 | 0.03253 | 5.24 | 0.16933 |
| lca-miR-17-3p_st | 5.316 | 0.03633 | 7.33 | 0.16951 |
| hsa-miR-486-5p_st | 21.543 | 0.15432 | 25.60 | 0.16955 |
| tni-miR-26_st | 1.188 | 0.17700 | 2.09 | 0.16959 |
| mne-miR-19b_st | 3.273 | 0.00752 | 5.57 | 0.16961 |
| oan-miR-10a_st | 116.132 | 0.04318 | 212.10 | 0.16972 |
| ppa-miR-30b_st | 1.050 | 0.37351 | 2.44 | 0.17030 |
| ENSG00000200130_x_st | 1.532 | 0.08262 | 2.19 | 0.17073 |
| ACA36_x_st | 2.373 | 0.23440 | 2.89 | 0.17133 |
| U83_st | 0.886 | 0.66472 | 6.57 | 0.17143 |
| cfa-miR-19a_st | 2.530 | 0.05723 | 8.27 | 0.17144 |
| age-miR-30b_st | 1.259 | 0.13998 | 2.36 | 0.17152 |
| xtr-miR-155_st | 1.265 | 0.17171 | 2.63 | 0.17153 |
| dre-miR-363_st | 5.378 | 0.00497 | 4.63 | 0.17154 |
| ENSG00000239128_x_st | 1.660 | 0.15299 | 2.33 | 0.17201 |
| eca-miR-146a_st | 0.881 | 0.81034 | 3.06 | 0.17218 |
| oan-miR-30d_st | 1.306 | 0.04326 | 2.56 | 0.17222 |
| ptr-let-7f_st | 1.232 | 0.10319 | 2.31 | 0.17237 |
| snR39B_st | 0.917 | 0.84069 | 3.01 | 0.17273 |
| ptr-miR-196b_st | 63.497 | 0.00624 | 20.44 | 0.17306 |
| ssc-miR-363_st | 3.649 | 0.01342 | 3.28 | 0.17331 |
| U25_st | 0.814 | 0.50969 | 10.12 | 0.17332 |
| mmu-miR-146b_st | 0.800 | 0.49491 | 3.73 | 0.17370 |
| hsa-miR-100_st | 18.999 | 0.05358 | 39.72 | 0.17397 |
| mml-miR-26a_st | 1.138 | 0.16922 | 2.24 | 0.17401 |
| fru-miR-10c_st | 4.693 | 0.19978 | 5.58 | 0.17419 |
| rno-miR-26a_st | 1.131 | 0.16936 | 2.28 | 0.17431 |
| rno-miR-100_st | 31.708 | 0.00872 | 48.77 | 0.17446 |
| U17a_x_st | 2.326 | 0.21350 | 3.99 | 0.17457 |
| rno-miR-128_st | 1.648 | 0.11527 | 2.42 | 0.17538 |
| xtr-miR-10a_st | 171.092 | 0.03491 | 177.79 | 0.17560 |
| mdo-miR-10a_st | 97.077 | 0.02708 | 98.18 | 0.17565 |
| oan-miR-100_st | 21.627 | 0.03246 | 70.63 | 0.17582 |
| mml-miR-17-3p_st | 9.246 | 0.00012 | 16.62 | 0.17585 |
| mgh18S-121_st | 0.917 | 0.70752 | 10.22 | 0.17598 |
| hsa-miR-1201_st | 1.527 | 0.34793 | 5.25 | 0.17615 |
| ssc-miR-100_st | 13.206 | 0.04191 | 71.70 | 0.17616 |
| nvi-miR-100_st | 34.493 | 0.03103 | 47.67 | 0.17625 |
| dan-miR-10_st | 164.222 | 0.05253 | 160.10 | 0.17653 |
| mml-miR-551b_st | 41.409 | 0.01319 | 33.92 | 0.17678 |
| U3_s_st | 2.467 | 0.22510 | 32.72 | 0.17713 |
| ENSG00000201009_s_st | 1.453 | 0.21770 | 21.82 | 0.17732 |
| fru-miR-19a_st | 3.074 | 0.04570 | 4.64 | 0.17742 |
| ppy-miR-423-3p_st | 0.900 | 0.19776 | 2.87 | 0.17754 |
| tni-miR-19b_st | 3.271 | 0.00929 | 4.98 | 0.17776 |
| ptr-miR-146a_st | 0.931 | 0.88264 | 2.87 | 0.17780 |
| bta-miR-196b_st | 57.472 | 0.05871 | 39.44 | 0.17781 |
| oan-miR-19a_st | 2.760 | 0.03635 | 6.43 | 0.17785 |
| dre-miR-10a_st | 113.223 | 0.04779 | 183.19 | 0.17804 |
| ssc-miR-17-3p_st | 3.553 | 0.01912 | 12.25 | 0.17888 |
| bta-miR-93_st | 1.681 | 0.08450 | 3.10 | 0.17902 |
| ptr-miR-19b_st | 2.930 | 0.00169 | 4.86 | 0.17918 |
| eca-miR-146b-5p_st | 0.782 | 0.15000 | 3.21 | 0.17931 |
| bta-miR-1248_st | 1.737 | 0.54191 | 2.84 | 0.17973 |
| U13_x_st | 2.587 | 0.08938 | 16.29 | 0.17986 |
| U42A_st | 1.461 | 0.24963 | 4.12 | 0.18030 |
| dpe-miR-10_st | 80.159 | 0.03884 | 92.18 | 0.18050 |
| dme-miR-10-5p_st | 86.048 | 0.03183 | 138.60 | 0.18074 |
| ptr-miR-30d_st | 1.221 | 0.49276 | 2.10 | 0.18115 |
| ENSG00000238645_x_st | 1.574 | 0.11695 | 4.89 | 0.18173 |
| hvt-miR-H14-star_st | 3.253 | 0.04409 | 4.34 | 0.18188 |
| hsa-miR-25_st | 1.464 | 0.13865 | 2.25 | 0.18224 |
| xtr-miR-30d_st | 1.122 | 0.38497 | 2.27 | 0.18231 |
| U90_st | 1.407 | 0.53368 | 5.85 | 0.18250 |
| aga-miR-10_st | 86.633 | 0.04293 | 97.87 | 0.18282 |
| HBII-142_x_st | 0.603 | 0.20790 | 15.58 | 0.18305 |
| tgu-let-7f_st | 1.292 | 0.30909 | 2.71 | 0.18319 |
| U51_x_st | 1.218 | 0.53954 | 2.78 | 0.18373 |
| U105_st | 0.579 | 0.14000 | 2.77 | 0.18375 |
| U20_st | 2.061 | 0.15809 | 13.06 | 0.18377 |
| rno-miR-19a_st | 3.437 | 0.06330 | 4.39 | 0.18383 |
| ENSG00000251940_s_st | 3.451 | 0.27228 | 3.04 | 0.18394 |
| age-miR-214_st | 0.929 | 0.68974 | 2.81 | 0.18436 |
| dre-miR-221_st | 1.649 | 0.02378 | 2.65 | 0.18464 |
| hp_hsa-mir-145_st | 0.746 | 0.47255 | 3.31 | 0.18474 |
| cfa-miR-26a_st | 1.172 | 0.20054 | 2.18 | 0.18499 |
| eca-miR-192_st | 0.234 | 0.12794 | 2.65 | 0.18531 |
| oan-miR-10a-star_st | 6.069 | 0.05415 | 5.47 | 0.18536 |
| ACA1_x_st | 2.207 | 0.23150 | 4.51 | 0.18543 |
| U59B_x_st | 0.921 | 0.67267 | 6.20 | 0.18574 |
| xtr-miR-363-3p_st | 4.510 | 0.00266 | 3.87 | 0.18655 |
| oan-miR-26_st | 1.134 | 0.27534 | 2.13 | 0.18656 |
| aae-miR-10_st | 108.549 | 0.02361 | 97.80 | 0.18660 |
| xtr-miR-26_st | 1.193 | 0.24647 | 2.20 | 0.18777 |
| gga-miR-146c_st | 1.162 | 0.84537 | 3.55 | 0.18781 |
| HBII-296A_x_st | 1.301 | 0.41405 | 2.49 | 0.18788 |
| xtr-miR-100_st | 24.062 | 0.01309 | 49.75 | 0.18791 |
| U56_x_st | 1.342 | 0.08421 | 16.50 | 0.18828 |
| U53_st | 2.272 | 0.24296 | 15.50 | 0.18830 |
| U44_st | 0.984 | 0.89439 | 12.64 | 0.18832 |
| ggo-miR-10a_st | 100.707 | 0.04390 | 140.84 | 0.18863 |
| dmo-miR-10_st | 120.363 | 0.01643 | 142.72 | 0.18869 |
| xtr-miR-93b_st | 1.959 | 0.07023 | 2.35 | 0.18908 |
| age-miR-17-3p_st | 9.183 | 0.00422 | 6.24 | 0.18925 |
| U68_x_st | 1.812 | 0.17998 | 9.32 | 0.18927 |
| dre-miR-30b_st | 1.264 | 0.28240 | 2.44 | 0.18942 |
| lla-miR-19a_st | 2.264 | 0.04489 | 4.69 | 0.18946 |
| ppy-miR-551b_st | 33.121 | 0.00001 | 33.96 | 0.18950 |
| U48_st | 0.806 | 0.56534 | 23.53 | 0.18983 |
| mne-miR-93_st | 2.045 | 0.07855 | 2.65 | 0.18999 |
| mdo-miR-18_st | 3.207 | 0.02109 | 5.49 | 0.19008 |
| rno-miR-17-3p_st | 4.841 | 0.07070 | 4.98 | 0.19038 |
| bta-miR-146a_st | 1.612 | 0.17288 | 3.20 | 0.19082 |
| mdo-miR-196b_st | 25.103 | 0.09056 | 8.79 | 0.19088 |
| ptr-miR-17-3p_st | 11.943 | 0.01850 | 8.19 | 0.19107 |
| mmu-miR-106b_st | 1.707 | 0.06053 | 2.20 | 0.19116 |
| odi-let-7a_st | 1.683 | 0.25371 | 2.07 | 0.19125 |
| ACA7_s_st | 1.611 | 0.34539 | 4.29 | 0.19125 |
| U71c_x_st | 3.831 | 0.02509 | 3.05 | 0.19144 |
| ssc-miR-26a_st | 1.171 | 0.09842 | 2.15 | 0.19161 |
| rno-miR-146b_st | 0.812 | 0.31779 | 2.85 | 0.19213 |
| dre-let-7f_st | 1.300 | 0.22481 | 2.39 | 0.19224 |
| ACA48_x_st | 1.581 | 0.31203 | 5.98 | 0.19236 |
| mdo-miR-26_st | 1.165 | 0.21048 | 2.14 | 0.19239 |
| ACA23_st | 2.849 | 0.14492 | 2.51 | 0.19242 |
| odi-miR-92b_st | 2.672 | 0.05782 | 2.64 | 0.19277 |
| mmu-miR-18a-star_st | 5.010 | 0.14465 | 5.97 | 0.19301 |
| mmu-miR-25_st | 2.103 | 0.01296 | 3.42 | 0.19336 |
| isc-miR-10_st | 140.993 | 0.05849 | 156.17 | 0.19337 |
| bta-miR-10a_st | 126.620 | 0.04296 | 199.01 | 0.19380 |
| bta-miR-30b-5p_st | 0.798 | 0.09590 | 2.81 | 0.19391 |
| tgu-miR-146b_st | 0.732 | 0.26216 | 3.64 | 0.19426 |
| dre-miR-26a_st | 1.147 | 0.10292 | 2.17 | 0.19432 |
| rno-miR-335_st | 8.548 | 0.04661 | 21.24 | 0.19451 |
| cfa-miR-130b_st | 3.229 | 0.01065 | 4.30 | 0.19484 |
| SNORD121B_x_st | 1.401 | 0.21474 | 3.74 | 0.19496 |
| ggo-miR-10b_st | 8.454 | 0.26073 | 6.50 | 0.19504 |
| tgu-miR-17a_st | 7.268 | 0.00187 | 12.32 | 0.19549 |
| dvi-miR-10_st | 110.964 | 0.03212 | 109.24 | 0.19555 |
| ppa-miR-93_st | 1.715 | 0.02769 | 3.32 | 0.19668 |
| U36A_x_st | 1.818 | 0.13504 | 4.37 | 0.19714 |
| ggo-miR-181a-star_st | 4.904 | 0.08237 | 5.65 | 0.19717 |
| mml-miR-196b_st | 36.480 | 0.01196 | 16.13 | 0.19717 |
| ssc-miR-10a_st | 113.986 | 0.00924 | 172.89 | 0.19744 |
| dre-miR-26b_st | 0.983 | 0.92398 | 2.03 | 0.19762 |
| ppy-miR-100_st | 17.094 | 0.00993 | 51.54 | 0.19766 |
| hp_hsa-mir-181b-1_st | 1.624 | 0.24020 | 2.44 | 0.19792 |
| eca-miR-551b_st | 31.124 | 0.01093 | 23.17 | 0.19796 |
| ACA1_s_st | 4.443 | 0.00734 | 2.93 | 0.19797 |
| cfa-miR-128_st | 1.400 | 0.25423 | 3.22 | 0.19820 |
| gga-miR-100_st | 16.071 | 0.01094 | 42.99 | 0.19833 |
| eca-miR-19a_st | 2.527 | 0.06880 | 5.45 | 0.19897 |
| U38B_x_st | 0.829 | 0.30544 | 7.60 | 0.19966 |
| mne-miR-17-3p_st | 9.777 | 0.00058 | 13.60 | 0.19969 |
| bta-miR-340_st | 2.756 | 0.36822 | 2.71 | 0.20089 |
| mmu-miR-19a_st | 3.318 | 0.05301 | 5.05 | 0.20137 |
| hsa-miR-30d_st | 1.027 | 0.70753 | 2.11 | 0.20192 |
| U17a_st | 2.282 | 0.15654 | 3.11 | 0.20273 |
| ptr-miR-100_st | 29.980 | 0.02847 | 55.70 | 0.20276 |
| bmo-miR-10_st | 137.347 | 0.02280 | 114.41 | 0.20295 |
| sla-miR-19a_st | 3.719 | 0.05244 | 5.34 | 0.20322 |
| rno-miR-363_st | 4.174 | 0.01571 | 3.67 | 0.20344 |
| gga-miR-26a_st | 1.231 | 0.06472 | 2.21 | 0.20370 |
| hp_mmu-mir-222_st | 2.135 | 0.10202 | 2.70 | 0.20383 |
| U84_st | 0.651 | 0.25597 | 5.19 | 0.20397 |
| mgU6-53_x_st | 1.266 | 0.61831 | 3.09 | 0.20406 |
| dre-miR-181a-star_st | 4.782 | 0.06802 | 3.63 | 0.20453 |
| ame-miR-929_st | 1.373 | 0.30663 | 2.13 | 0.20465 |
| mml-miR-10b_st | 24.374 | 0.08932 | 18.50 | 0.20472 |
| mml-miR-181a-star_st | 10.291 | 0.04424 | 2.87 | 0.20525 |
| xtr-miR-19a_st | 2.723 | 0.03919 | 4.85 | 0.20543 |
| ACA8_x_st | 1.812 | 0.41024 | 6.74 | 0.20545 |
| ssc-let-7e_st | 1.079 | 0.90815 | 2.01 | 0.20607 |
| xtr-miR-93a_st | 1.959 | 0.05717 | 2.85 | 0.20613 |
| dre-miR-25_st | 1.970 | 0.01435 | 2.78 | 0.20627 |
| mmu-miR-222_st | 1.986 | 0.07374 | 2.67 | 0.20628 |
| mne-miR-26a_st | 1.147 | 0.25214 | 2.11 | 0.20639 |
| dgr-miR-10_st | 85.169 | 0.02798 | 173.34 | 0.20641 |
| hp_hsa-mir-146b_x_st | 2.316 | 0.46895 | 2.13 | 0.20659 |
| tni-miR-30d_st | 1.450 | 0.00842 | 2.06 | 0.20694 |
| HBII-85-15_x_st | 1.702 | 0.18262 | 4.39 | 0.20696 |
| ssc-miR-186_st | 1.102 | 0.84383 | 3.09 | 0.20716 |
| tgu-miR-363_st | 5.414 | 0.04468 | 3.22 | 0.20718 |
| ssc-miR-128_st | 1.940 | 0.11386 | 2.72 | 0.20733 |
| ggo-miR-19a_st | 2.932 | 0.01601 | 4.30 | 0.20765 |
| U18A_st | 0.760 | 0.09757 | 3.25 | 0.20784 |
| hp_mmu-mir-2135-5_x_st | 1.117 | 0.38707 | 2.18 | 0.20796 |
| sla-miR-128_st | 1.119 | 0.83341 | 3.63 | 0.20824 |
| hsa-miR-146b-5p_st | 0.935 | 0.84329 | 2.89 | 0.20847 |
| ACA27_x_st | 2.597 | 0.04314 | 2.58 | 0.20857 |
| ENSG00000221750_st | 8.372 | 0.06903 | 19.78 | 0.20869 |
| hsa-miR-361-5p_st | 1.087 | 0.25507 | 2.66 | 0.20878 |
| ssc-miR-486_st | 63.116 | 0.08049 | 722.14 | 0.20878 |
| tni-miR-19a_st | 2.765 | 0.00629 | 5.37 | 0.20906 |
| rno-miR-10a-5p_st | 175.245 | 0.01588 | 204.66 | 0.20989 |
| mne-miR-18_st | 3.314 | 0.10729 | 5.76 | 0.21019 |
| xla-miR-18_st | 2.789 | 0.10264 | 5.94 | 0.21042 |
| mmu-miR-30a_st | 1.633 | 0.06053 | 2.42 | 0.21088 |
| bta-miR-2424_st | 0.643 | 0.11871 | 5.30 | 0.21092 |
| dse-miR-10_st | 107.374 | 0.07147 | 112.94 | 0.21107 |
| U95_st | 0.745 | 0.23621 | 5.01 | 0.21124 |
| gga-miR-19a_st | 2.287 | 0.02441 | 4.35 | 0.21180 |
| dre-miR-101a_st | 1.125 | 0.78063 | 3.09 | 0.21183 |
| sla-miR-18_st | 5.155 | 0.03998 | 5.39 | 0.21234 |
| dwi-miR-10_st | 74.108 | 0.05932 | 116.01 | 0.21267 |
| lla-miR-101_st | 1.280 | 0.65659 | 4.08 | 0.21299 |
| rno-miR-101a_st | 1.602 | 0.23962 | 5.22 | 0.21347 |
| U28_st | 1.335 | 0.27933 | 7.28 | 0.21350 |
| gga-miR-146b_st | 0.717 | 0.19102 | 3.17 | 0.21358 |
| ssc-miR-19a_st | 3.577 | 0.01052 | 6.11 | 0.21482 |
| tgu-miR-155_st | 1.056 | 0.80811 | 3.38 | 0.21499 |
| hsa-miR-26a_st | 1.191 | 0.20777 | 2.10 | 0.21499 |
| hsa-miR-886-3p_st | 8.276 | 0.06939 | 5.92 | 0.21530 |
| hsa-miR-19a_st | 2.928 | 0.02670 | 4.05 | 0.21575 |
| rno-miR-29c-star_st | 0.568 | 0.45511 | 3.38 | 0.21576 |
| ptr-miR-26a_st | 1.148 | 0.22457 | 2.26 | 0.21577 |
| dps-miR-100_st | 2.153 | 0.00821 | 2.15 | 0.21611 |
| lla-miR-17-3p_st | 5.223 | 0.02701 | 8.59 | 0.21614 |
| mmu-miR-486_st | 3.862 | 0.04756 | 316.42 | 0.21640 |
| HBII-166_st | 0.996 | 0.99018 | 3.53 | 0.21691 |
| cfa-miR-146b_st | 0.759 | 0.23373 | 2.66 | 0.21701 |
| oan-miR-551_st | 33.612 | 0.01107 | 30.30 | 0.21737 |
| rno-miR-106b-star_st | 2.386 | 0.09516 | 3.57 | 0.21739 |
| bta-miR-25_st | 1.861 | 0.03046 | 2.54 | 0.21754 |
| tgu-miR-146a-star_st | 1.231 | 0.62097 | 4.25 | 0.21781 |
| HBII-85-17_x_st | 1.600 | 0.21625 | 4.52 | 0.21782 |
| bta-miR-106b_st | 1.294 | 0.08337 | 2.52 | 0.21793 |
| eca-miR-155_st | 1.092 | 0.64002 | 3.04 | 0.21825 |
| ppy-miR-17-3p_st | 7.719 | 0.02737 | 9.60 | 0.21863 |
| mmu-miR-551b_st | 38.058 | 0.03434 | 23.58 | 0.21890 |
| dya-miR-10_st | 81.044 | 0.06714 | 90.82 | 0.21931 |
| bta-miR-551b_st | 65.929 | 0.00712 | 65.23 | 0.21978 |
| bta-miR-146b_st | 0.905 | 0.49331 | 2.68 | 0.22060 |
| ggo-miR-17-3p_st | 5.207 | 0.04712 | 5.95 | 0.22077 |
| ACA5_st | 1.916 | 0.36318 | 10.05 | 0.22104 |
| ppa-miR-17-3p_st | 5.468 | 0.00853 | 8.47 | 0.22134 |
| mmu-miR-26a_st | 1.137 | 0.08725 | 2.08 | 0.22167 |
| mml-miR-324-3p_st | 3.106 | 0.10460 | 3.54 | 0.22169 |
| ACA55_st | 1.585 | 0.04567 | 3.90 | 0.22176 |
| mdo-miR-101_st | 1.481 | 0.45721 | 4.31 | 0.22180 |
| 14qll-1_st | 3.533 | 0.04515 | 3.23 | 0.22217 |
| bta-miR-2439_st | 0.784 | 0.35352 | 2.84 | 0.22254 |
| ENSG00000206785_s_st | 2.138 | 0.00651 | 8.33 | 0.22271 |
| rno-miR-30c_st | 0.992 | 0.96909 | 2.02 | 0.22292 |
| U46_st | 1.527 | 0.50160 | 14.25 | 0.22349 |
| tgu-miR-18b_st | 3.132 | 0.06742 | 4.77 | 0.22356 |
| U71b_st | 2.796 | 0.17646 | 5.64 | 0.22439 |
| oan-miR-146b_st | 0.872 | 0.45989 | 3.40 | 0.22458 |
| 14qll-12_x_st | 3.017 | 0.01954 | 2.03 | 0.22458 |
| ACA57_x_st | 1.573 | 0.11497 | 5.69 | 0.22509 |
| lla-miR-100_st | 32.593 | 0.03420 | 42.95 | 0.22544 |
| hsa-miR-18a-star_st | 6.585 | 0.08835 | 6.79 | 0.22548 |
| rno-miR-106b_st | 2.060 | 0.06582 | 2.44 | 0.22573 |
| ppy-miR-101_st | 1.307 | 0.59902 | 2.82 | 0.22579 |
| hsa-miR-16-2-star_st | 3.251 | 0.07270 | 3.92 | 0.22635 |
| dre-miR-146b_st | 0.961 | 0.95600 | 4.12 | 0.22638 |
| ENSG00000200879_st | 0.876 | 0.60268 | 9.56 | 0.22646 |
| ptr-miR-324_st | 3.016 | 0.05349 | 5.98 | 0.22746 |
| oan-miR-18_st | 3.272 | 0.04684 | 4.02 | 0.22747 |
| xtr-miR-17-3p_st | 8.583 | 0.00740 | 6.66 | 0.22793 |
| mml-let-7f_st | 1.206 | 0.07711 | 2.41 | 0.22824 |
| U103_s_st | 0.723 | 0.42340 | 2.11 | 0.22839 |
| hp_hsa-mir-106b_st | 1.603 | 0.22065 | 2.96 | 0.22845 |
| ptr-miR-19a_st | 3.283 | 0.07698 | 3.93 | 0.22890 |
| xtr-miR-130b_st | 4.543 | 0.01701 | 4.85 | 0.22895 |
| ENSG00000201199_s_st | 2.338 | 0.19702 | 2.76 | 0.22897 |
| ppy-miR-128_st | 1.751 | 0.03513 | 2.82 | 0.22898 |
| ppa-miR-19a_st | 2.804 | 0.02259 | 4.36 | 0.22899 |
| ENSG00000207187_s_st | 2.405 | 0.08371 | 3.05 | 0.22912 |
| hsa-miR-17-star_st | 4.816 | 0.06278 | 20.32 | 0.22983 |
| v11_hsa-miR-768-5p_st | 0.367 | 0.11328 | 4.07 | 0.23024 |
| hp_rno-mir-17-1_st | 2.621 | 0.00119 | 2.95 | 0.23059 |
| age-miR-93_st | 2.267 | 0.01622 | 2.68 | 0.23064 |
| mml-miR-18b_st | 2.915 | 0.03951 | 3.68 | 0.23169 |
| ptr-miR-1248_st | 1.103 | 0.91077 | 4.30 | 0.23196 |
| dsi-let-7_st | 1.221 | 0.11033 | 2.08 | 0.23201 |
| dre-miR-10b_st | 29.479 | 0.12727 | 19.14 | 0.23306 |
| cfa-miR-93_st | 1.838 | 0.01039 | 2.46 | 0.23318 |
| eca-miR-411_st | 1.418 | 0.03010 | 2.01 | 0.23352 |
| bta-miR-19a_st | 4.280 | 0.00899 | 4.21 | 0.23357 |
| ENSG00000238581_x_st | 1.702 | 0.07030 | 4.98 | 0.23412 |
| der-miR-100_st | 2.771 | 0.36959 | 2.57 | 0.23416 |
| U71d_x_st | 2.107 | 0.11757 | 3.82 | 0.23440 |
| ACA24_x_st | 4.426 | 0.09662 | 11.54 | 0.23484 |
| ggo-miR-30d_st | 1.436 | 0.10699 | 2.27 | 0.23522 |
| tgu-miR-130b_st | 3.167 | 0.07463 | 5.39 | 0.23560 |
| oan-miR-101_st | 1.245 | 0.44079 | 3.89 | 0.23572 |
| ENSG00000239123_st | 0.863 | 0.49719 | 2.26 | 0.23662 |
| mdo-miR-10b_st | 13.108 | 0.09951 | 9.47 | 0.23668 |
| ssc-miR-374b_st | 1.814 | 0.50820 | 5.72 | 0.23677 |
| ACA50_st | 2.397 | 0.11114 | 4.58 | 0.23751 |
| rno-miR-423_st | 0.943 | 0.72371 | 2.85 | 0.23817 |
| ggo-miR-30a-5p_st | 1.148 | 0.48663 | 2.23 | 0.23866 |
| HBII-85-29_x_st | 2.363 | 0.34444 | 2.69 | 0.23910 |
| dme-miR-310_st | 2.498 | 0.34717 | 2.56 | 0.23930 |
| ssc-miR-1_st | 1.658 | 0.01153 | 4.21 | 0.24194 |
| eca-miR-10b_st | 31.880 | 0.10203 | 24.43 | 0.24440 |
| gga-miR-181a-star_st | 9.958 | 0.01497 | 4.92 | 0.24443 |
| oan-miR-128_st | 1.719 | 0.27445 | 2.26 | 0.24477 |
| ENSG00000207217_st | 1.359 | 0.21207 | 3.24 | 0.24479 |
| oan-miR-20a-2-star_st | 11.441 | 0.02720 | 4.24 | 0.24481 |
| bta-miR-151_st | 1.360 | 0.51439 | 6.36 | 0.24526 |
| U41_x_st | 2.429 | 0.08874 | 4.75 | 0.24547 |
| ACA5_x_st | 1.515 | 0.23016 | 4.89 | 0.24548 |
| cfa-miR-381_st | 0.812 | 0.01425 | 2.03 | 0.24599 |
| ssc-miR-146b_st | 1.201 | 0.31120 | 2.53 | 0.24665 |
| xtr-miR-18b_st | 2.771 | 0.00246 | 2.94 | 0.24825 |
| gga-miR-1582_st | 0.867 | 0.69965 | 2.61 | 0.24831 |
| ENSG00000252840_x_st | 2.073 | 0.29765 | 2.18 | 0.24871 |
| mne-miR-19a_st | 3.250 | 0.06386 | 3.75 | 0.24928 |
| U83A_x_st | 2.444 | 0.23800 | 3.76 | 0.24941 |
| HBI-61_st | 1.221 | 0.23326 | 2.67 | 0.24964 |
| oan-miR-196b_st | 18.442 | 0.06590 | 4.76 | 0.24997 |
| ENSG00000206903_x_st | 4.363 | 0.12232 | 2.69 | 0.25017 |
| hsa-miR-1_st | 1.651 | 0.27103 | 3.29 | 0.25046 |
| ppy-miR-98_st | 0.963 | 0.83558 | 4.47 | 0.25091 |
| HBII-85-20_x_st | 2.315 | 0.01342 | 2.76 | 0.25097 |
| hsa-miR-4324_st | 3.413 | 0.19129 | 7.29 | 0.25113 |
| age-miR-101_st | 1.444 | 0.25690 | 4.22 | 0.25135 |
| age-miR-19a_st | 3.701 | 0.02378 | 3.65 | 0.25163 |
| mml-miR-609_st | 1.272 | 0.48975 | 2.27 | 0.25224 |
| tgu-miR-100_st | 27.934 | 0.08711 | 33.75 | 0.25229 |
| eca-miR-18b_st | 2.849 | 0.03568 | 5.22 | 0.25254 |
| cfa-miR-551b_st | 43.263 | 0.02566 | 44.39 | 0.25276 |
| ENSG00000208308_x_st | 1.914 | 0.22657 | 2.89 | 0.25412 |
| eca-miR-26a_st | 1.198 | 0.11944 | 2.28 | 0.25419 |
| ENSG00000200385_st | 1.315 | 0.19707 | 2.25 | 0.25454 |
| hsa-miR-130b-star_st | 2.888 | 0.28154 | 2.91 | 0.25480 |
| ssc-miR-99b_st | 0.534 | 0.28973 | 4.56 | 0.25680 |
| dre-miR-130b_st | 4.030 | 0.00642 | 4.77 | 0.25693 |
| dre-miR-222b_st | 2.734 | 0.19471 | 5.15 | 0.25712 |
| bmo-miR-13b-star_st | 1.034 | 0.29381 | 2.15 | 0.25767 |
| mdo-miR-551b_st | 29.803 | 0.00606 | 28.99 | 0.25777 |
| ptr-miR-128_st | 0.984 | 0.95960 | 2.82 | 0.25810 |
| U71d_st | 3.386 | 0.00627 | 7.18 | 0.25816 |
| eca-miR-130b_st | 3.887 | 0.02408 | 7.00 | 0.25830 |
| bta-miR-17-3p_st | 12.853 | 0.01004 | 5.61 | 0.25849 |
| HBII-85-25_s_st | 2.272 | 0.04711 | 2.05 | 0.25858 |
| mml-miR-331-3p_st | 0.822 | 0.62668 | 7.02 | 0.25865 |
| dgr-miR-309_st | 1.396 | 0.46194 | 2.01 | 0.25878 |
| U96b_x_st | 1.263 | 0.54695 | 4.74 | 0.25883 |
| bna-miR397b_st | 1.154 | 0.45839 | 2.40 | 0.25912 |
| gga-miR-18b_st | 2.982 | 0.00991 | 2.87 | 0.25943 |
| eca-miR-93_st | 2.025 | 0.06815 | 2.81 | 0.25978 |
| mml-miR-130b_st | 3.854 | 0.04251 | 4.57 | 0.26005 |
| HBII-336_st | 2.180 | 0.04475 | 5.51 | 0.26015 |
| ptr-miR-146b_st | 0.903 | 0.71772 | 2.74 | 0.26064 |
| ssc-miR-10b_st | 10.424 | 0.24696 | 8.29 | 0.26066 |
| lla-miR-26a_st | 1.143 | 0.25266 | 2.07 | 0.26070 |
| cfa-miR-155_st | 1.090 | 0.67680 | 3.35 | 0.26133 |
| mmu-miR-1983_st | 1.969 | 0.12875 | 2.79 | 0.26144 |
| HBII-296B_st | 1.059 | 0.68918 | 3.70 | 0.26164 |
| mmu-miR-18a_st | 3.431 | 0.01511 | 3.56 | 0.26219 |
| U8_x_st | 1.958 | 0.21164 | 16.69 | 0.26221 |
| cfa-miR-199_st | 2.413 | 0.01399 | 2.31 | 0.26223 |
| ptr-miR-345_st | 1.067 | 0.69265 | 6.38 | 0.26242 |
| ENSG00000212149_x_st | 1.193 | 0.60544 | 2.53 | 0.26293 |
| HBII-419_st | 1.161 | 0.63166 | 2.44 | 0.26307 |
| U69_st | 2.715 | 0.13421 | 2.02 | 0.26386 |
| ACA51_x_st | 2.350 | 0.25863 | 10.81 | 0.26421 |
| mmu-miR-1968_st | 1.100 | 0.34777 | 3.29 | 0.26435 |
| cfa-miR-18b_st | 2.427 | 0.06875 | 3.04 | 0.26442 |
| ACA25_x_st | 2.486 | 0.13998 | 4.32 | 0.26473 |
| U80_st | 0.790 | 0.57636 | 4.32 | 0.26509 |
| ENSG00000201042_st | 1.475 | 0.22543 | 2.17 | 0.26613 |
| SNORA11B_x_st | 2.015 | 0.27987 | 3.82 | 0.26631 |
| mmu-miR-17-star_st | 5.488 | 0.03695 | 2.73 | 0.26727 |
| ptr-miR-10b_st | 32.741 | 0.05748 | 18.01 | 0.26736 |
| tni-let-7e_st | 1.234 | 0.52962 | 2.52 | 0.26774 |
| ppy-miR-363_st | 6.994 | 0.01536 | 3.68 | 0.26807 |
| mmu-miR-93-star_st | 2.876 | 0.25709 | 3.26 | 0.26837 |
| gga-miR-3534_st | 1.194 | 0.72717 | 2.08 | 0.26846 |
| U103B_s_st | 0.950 | 0.88278 | 4.03 | 0.26894 |
| ACA45_st | 1.622 | 0.08044 | 2.48 | 0.26999 |
| cel-miR-1_st | 2.734 | 0.06030 | 3.14 | 0.27159 |
| mdo-miR-128_st | 1.806 | 0.04456 | 4.48 | 0.27174 |
| xtr-miR-146_st | 0.843 | 0.68822 | 2.65 | 0.27211 |
| ssc-miR-151-5p_st | 2.759 | 0.08210 | 4.38 | 0.27214 |
| cfa-miR-363_st | 2.773 | 0.03377 | 3.39 | 0.27338 |
| U14B_st | 1.518 | 0.09877 | 2.98 | 0.27350 |
| U61_st | 0.782 | 0.14764 | 4.55 | 0.27390 |
| mmu-miR-363_st | 3.896 | 0.02385 | 2.56 | 0.27421 |
| gga-miR-551_st | 43.923 | 0.03815 | 26.62 | 0.27517 |
| SNORA11B_st | 2.123 | 0.09978 | 2.60 | 0.27603 |
| rno-miR-30a_st | 1.258 | 0.11295 | 2.14 | 0.27646 |
| aga-let-7_st | 1.474 | 0.56184 | 2.83 | 0.27662 |
| ppy-miR-19a_st | 3.518 | 0.03030 | 3.61 | 0.27806 |
| oan-miR-10b_st | 11.493 | 0.28146 | 5.97 | 0.27843 |
| tni-miR-10c_st | 2.824 | 0.10046 | 4.69 | 0.27918 |
| tni-miR-128_st | 1.194 | 0.23721 | 2.34 | 0.27984 |
| hsa-miR-101_st | 0.968 | 0.81497 | 5.33 | 0.27992 |
| mmu-miR-374_st | 1.653 | 0.48940 | 4.82 | 0.28080 |
| xtr-miR-10b_st | 9.932 | 0.23373 | 7.33 | 0.28085 |
| ACA38_st | 1.622 | 0.21388 | 3.35 | 0.28122 |
| mdo-miR-17-3p_st | 5.752 | 0.01603 | 7.23 | 0.28145 |
| gga-miR-30d_st | 1.053 | 0.35840 | 2.12 | 0.28162 |
| fru-miR-101a_st | 1.524 | 0.33664 | 5.58 | 0.28277 |
| hsa-miR-130b_st | 4.097 | 0.03634 | 3.68 | 0.28287 |
| bta-miR-374b_st | 1.347 | 0.69157 | 3.69 | 0.28315 |
| ACA20_st | 1.190 | 0.82366 | 5.11 | 0.28355 |
| oan-miR-363_st | 3.907 | 0.02773 | 2.96 | 0.28419 |
| ssc-miR-101_st | 0.711 | 0.13251 | 3.31 | 0.28447 |
| bta-miR-10b_st | 14.929 | 0.14690 | 23.97 | 0.28557 |
| dre-miR-18a_st | 3.574 | 0.05970 | 3.46 | 0.28683 |
| gga-miR-10b_st | 9.823 | 0.11185 | 6.47 | 0.28718 |
| bta-miR-127_st | 3.845 | 0.31312 | 4.97 | 0.28770 |
| ssc-miR-130b_st | 4.241 | 0.02961 | 4.33 | 0.28807 |
| ptr-miR-551a_st | 2.589 | 0.29435 | 2.28 | 0.28864 |
| mdo-miR-146b_st | 0.752 | 0.17648 | 3.01 | 0.28908 |
| snR38B_st | 1.786 | 0.24519 | 8.49 | 0.28971 |
| ptr-miR-18b_st | 2.596 | 0.02721 | 3.94 | 0.28984 |
| hsa-miR-424_st | 1.474 | 0.40830 | 2.58 | 0.29002 |
| ENSG00000206913_s_st | 1.702 | 0.14474 | 7.09 | 0.29045 |
| hsa-miR-551b_st | 42.611 | 0.01013 | 38.92 | 0.29087 |
| tni-miR-10b_st | 30.567 | 0.15984 | 16.75 | 0.29095 |
| ppy-miR-339-5p_st | 0.941 | 0.89792 | 14.90 | 0.29098 |
| rno-miR-25_st | 1.486 | 0.07080 | 2.33 | 0.29213 |
| bta-miR-363_st | 7.360 | 0.04382 | 2.89 | 0.29214 |
| hsa-miR-345_st | 1.642 | 0.31055 | 4.09 | 0.29222 |
| ggo-miR-101_st | 1.095 | 0.81172 | 4.01 | 0.29358 |
| tgu-miR-551_st | 31.728 | 0.01685 | 24.33 | 0.29397 |
| eca-miR-127_st | 3.442 | 0.13082 | 2.25 | 0.29524 |
| ppy-miR-886-3p_st | 8.166 | 0.07524 | 6.22 | 0.29546 |
| ppy-miR-10b_st | 29.025 | 0.10462 | 17.61 | 0.29553 |
| ptr-miR-886_st | 12.585 | 0.07604 | 4.82 | 0.29590 |
| E2_st | 2.793 | 0.10564 | 5.55 | 0.29627 |
| hp_hsa-mir-1291_s_st | 1.614 | 0.03654 | 5.22 | 0.29704 |
| U42B_st | 0.901 | 0.61235 | 2.11 | 0.29831 |
| ptr-miR-127_st | 4.780 | 0.07712 | 4.77 | 0.29893 |
| ACA46_st | 1.618 | 0.48012 | 3.37 | 0.29896 |
| dgr-miR-100_st | 2.575 | 0.12651 | 2.09 | 0.29953 |
| mmu-miR-128_st | 2.050 | 0.02883 | 2.03 | 0.30074 |
| U59A_st | 0.750 | 0.11777 | 2.06 | 0.30310 |
| fru-miR-10b_st | 32.045 | 0.06807 | 19.25 | 0.30432 |
| eca-miR-199b-5p_st | 2.089 | 0.10862 | 5.26 | 0.30440 |
| U84_x_st | 0.839 | 0.51404 | 3.48 | 0.30457 |
| ptr-miR-423_st | 0.667 | 0.37015 | 2.71 | 0.30509 |
| mml-miR-374b_st | 4.054 | 0.23327 | 3.74 | 0.30512 |
| mcmv-miR-M44-1_st | 1.347 | 0.73474 | 2.71 | 0.30522 |
| age-miR-18_st | 4.330 | 0.03496 | 3.08 | 0.30535 |
| ptr-miR-551b_st | 35.593 | 0.02626 | 32.46 | 0.30574 |
| bfl-let-7-1-as_st | 1.118 | 0.64540 | 2.30 | 0.30630 |
| mne-miR-101_st | 1.237 | 0.43915 | 2.92 | 0.30698 |
| hsa-miR-18a_st | 2.437 | 0.01622 | 4.28 | 0.30730 |
| eca-miR-101_st | 1.122 | 0.74164 | 3.36 | 0.30754 |
| hsa-miR-128_st | 1.561 | 0.21062 | 2.16 | 0.30799 |
| hp_hsa-mir-138-1_x_st | 1.108 | 0.63516 | 2.27 | 0.31035 |
| HBII-210_st | 1.040 | 0.87539 | 13.30 | 0.31090 |
| ENSG00000222489_st | 2.285 | 0.05694 | 3.46 | 0.31215 |
| U64_st | 1.955 | 0.11412 | 2.70 | 0.31342 |
| ppa-miR-181a-star_st | 5.809 | 0.04703 | 3.55 | 0.31374 |
| rno-miR-551b_st | 110.305 | 0.01574 | 43.93 | 0.31420 |
| hp_mmu-mir-26b_st | 0.386 | 0.11260 | 2.01 | 0.31442 |
| ppy-miR-10a_st | 10.337 | 0.03030 | 12.87 | 0.31583 |
| U8_st | 1.310 | 0.22342 | 23.24 | 0.31594 |
| ppy-miR-1271_st | 2.430 | 0.18723 | 2.82 | 0.31627 |
| xtr-miR-146b_st | 1.068 | 0.89492 | 3.17 | 0.31652 |
| fru-miR-128_st | 1.476 | 0.12577 | 2.70 | 0.31759 |
| sla-miR-101_st | 1.102 | 0.84342 | 3.41 | 0.31783 |
| mml-miR-339-5p_st | 0.739 | 0.48783 | 4.57 | 0.31915 |
| aae-let-7_st | 2.050 | 0.26946 | 3.98 | 0.31944 |
| lgi-miR-133_st | 2.425 | 0.34423 | 9.55 | 0.31999 |
| lla-miR-181a-star_st | 7.099 | 0.01144 | 2.21 | 0.32040 |
| mml-miR-151-5p_st | 1.062 | 0.92093 | 4.86 | 0.32109 |
| ENSG00000202335_x_st | 0.909 | 0.74514 | 2.44 | 0.32113 |
| mmu-miR-378-star_st | 1.247 | 0.51003 | 5.53 | 0.32121 |
| mml-miR-345_st | 1.437 | 0.28830 | 3.99 | 0.32241 |
| ssc-miR-361-3p_st | 0.574 | 0.36219 | 3.15 | 0.32248 |
| ptr-miR-1291_st | 7.632 | 0.32261 | 6.30 | 0.32297 |
| xtr-miR-1a_st | 2.289 | 0.06070 | 3.79 | 0.32395 |
| ppy-miR-486-5p_st | 8.350 | 0.06609 | 24.37 | 0.32699 |
| hsa-miR-181c-star_st | 3.941 | 0.05206 | 2.68 | 0.32755 |
| ggo-miR-98_st | 1.536 | 0.16333 | 4.58 | 0.32800 |
| ACA16_st | 1.049 | 0.93738 | 5.61 | 0.32817 |
| gga-miR-3535_st | 1.865 | 0.00123 | 13.07 | 0.32872 |
| eca-miR-769-5p_st | 0.897 | 0.82270 | 3.84 | 0.32876 |
| cfa-miR-17_st | 3.227 | 0.00374 | 6.55 | 0.32978 |
| gga-miR-1677_st | 0.958 | 0.91371 | 2.10 | 0.33069 |
| ENSG00000201388_s_st | 1.966 | 0.18110 | 2.25 | 0.33096 |
| mmu-miR-199a-3p_st | 2.010 | 0.20032 | 3.28 | 0.33290 |
| oan-miR-18-star_st | 1.451 | 0.09392 | 2.21 | 0.33404 |
| mmu-miR-101a_st | 0.668 | 0.05000 | 5.01 | 0.33435 |
| ppy-miR-106b_st | 1.362 | 0.14596 | 2.06 | 0.33553 |
| ACA10_s_st | 1.719 | 0.17623 | 2.98 | 0.33612 |
| ACA9_st | 2.356 | 0.00074 | 8.26 | 0.33628 |
| eca-miR-128_st | 1.416 | 0.21620 | 2.25 | 0.33635 |
| ptr-miR-1201_st | 1.377 | 0.33612 | 2.53 | 0.33657 |
| hsa-miR-324-3p_st | 1.583 | 0.39321 | 4.98 | 0.33725 |
| sja-miR-3503_st | 1.202 | 0.47253 | 2.38 | 0.33745 |
| ppy-miR-18b_st | 2.843 | 0.03230 | 3.90 | 0.33790 |
| fru-miR-1_st | 2.060 | 0.02347 | 3.66 | 0.33801 |
| gga-miR-130b_st | 4.468 | 0.00796 | 2.94 | 0.33833 |
| hsa-miR-99b_st | 0.561 | 0.39359 | 4.33 | 0.34016 |
| eca-miR-598_st | 1.741 | 0.24809 | 2.38 | 0.34025 |
| mtr-miR1510b-star_st | 1.290 | 0.36337 | 2.24 | 0.34134 |
| gga-miR-1808_st | 1.011 | 0.94215 | 2.08 | 0.34257 |
| dre-miR-10c_st | 2.439 | 0.07613 | 3.54 | 0.34279 |
| HBII-85-8_x_st | 1.575 | 0.19750 | 2.36 | 0.34428 |
| bta-miR-101_st | 1.213 | 0.30333 | 3.28 | 0.34462 |
| oan-miR-17-star_st | 12.691 | 0.02382 | 5.56 | 0.34477 |
| cin-miR-133_st | 1.321 | 0.59167 | 17.53 | 0.34570 |
| ENSG00000212532_st | 0.670 | 0.57223 | 9.37 | 0.34611 |
| mmu-miR-151-5p_st | 1.020 | 0.95478 | 4.79 | 0.34631 |
| sme-miR-67-3p_st | 1.450 | 0.50635 | 4.41 | 0.34661 |
| mml-miR-101_st | 0.767 | 0.47646 | 3.90 | 0.34663 |
| ppa-miR-10b_st | 10.625 | 0.12092 | 6.97 | 0.34680 |
| eca-miR-106b_st | 1.578 | 0.18787 | 2.01 | 0.34733 |
| U109_x_st | 1.927 | 0.29662 | 2.64 | 0.34756 |
| cfa-miR-151_st | 1.396 | 0.33862 | 3.66 | 0.34764 |
| bta-miR-2404_st | 3.259 | 0.21007 | 9.63 | 0.34858 |
| xtr-miR-214_st | 0.679 | 0.27418 | 2.22 | 0.34878 |
| sme-miR-10b_st | 6.045 | 0.30544 | 6.81 | 0.34884 |
| mtr-miR2585e_st | 0.740 | 0.19312 | 2.20 | 0.34936 |
| U67_x_st | 3.967 | 0.08937 | 3.38 | 0.34987 |
| ppa-miR-101_st | 0.974 | 0.91341 | 4.43 | 0.35326 |
| rno-miR-133a_st | 3.166 | 0.04956 | 9.38 | 0.35352 |
| hp_mmu-mir-1839_st | 2.470 | 0.11907 | 2.04 | 0.35551 |
| hsa-miR-1248_st | 1.774 | 0.44357 | 2.41 | 0.35676 |
| bta-miR-324_st | 0.956 | 0.89168 | 18.59 | 0.35733 |
| ppa-miR-1_st | 4.554 | 0.34897 | 2.42 | 0.35844 |
| ptr-miR-376c_st | 2.868 | 0.10475 | 4.73 | 0.35950 |
| v49_ENSG00000201373_st | 1.031 | 0.92807 | 2.25 | 0.35967 |
| gga-miR-18a_st | 2.889 | 0.02112 | 3.09 | 0.36073 |
| bmo-miR-2854_st | 0.959 | 0.48419 | 2.02 | 0.36200 |
| rno-miR-151_st | 1.503 | 0.32933 | 4.11 | 0.36285 |
| mmu-miR-10a-star_st | 6.757 | 0.10856 | 5.52 | 0.36290 |
| mmu-miR-133b_st | 2.341 | 0.33810 | 8.75 | 0.36368 |
| cre-miR908.3_st | 0.715 | 0.51295 | 4.19 | 0.36425 |
| rno-miR-382_st | 1.131 | 0.25539 | 3.08 | 0.36470 |
| eca-miR-199a-3p_st | 1.917 | 0.06432 | 2.78 | 0.36472 |
| lla-miR-18_st | 3.199 | 0.01302 | 2.94 | 0.36519 |
| cfa-miR-133a_st | 1.216 | 0.21006 | 12.03 | 0.36608 |
| eca-miR-454_st | 1.018 | 0.93076 | 2.90 | 0.36645 |
| hsa-miR-196b-star_st | 8.594 | 0.09998 | 4.49 | 0.36685 |
| cel-miR-269_st | 2.457 | 0.05654 | 2.91 | 0.36720 |
| U68_s_st | 3.713 | 0.09774 | 2.80 | 0.36758 |
| mmu-miR-98_st | 1.163 | 0.66587 | 4.93 | 0.36767 |
| hsa-miR-489_st | 1.963 | 0.16667 | 3.99 | 0.36809 |
| gga-miR-10a-star_st | 4.784 | 0.21690 | 3.76 | 0.36848 |
| mdo-miR-133a_st | 1.096 | 0.86947 | 8.46 | 0.36882 |
| rno-miR-374_st | 1.568 | 0.63111 | 3.28 | 0.36887 |
| ptr-miR-25_st | 3.241 | 0.19607 | 2.16 | 0.36925 |
| hsa-miR-378-star_st | 1.188 | 0.83817 | 3.45 | 0.37031 |
| HBII-420_st | 1.168 | 0.67113 | 2.53 | 0.37095 |
| ppy-miR-1286_st | 0.875 | 0.78928 | 2.30 | 0.37101 |
| rno-miR-10b_st | 7.974 | 0.23604 | 5.02 | 0.37164 |
| ptr-miR-301a_st | 1.104 | 0.49913 | 2.46 | 0.37179 |
| ssc-miR-133b_st | 1.368 | 0.33238 | 20.83 | 0.37231 |
| mdo-miR-199b_st | 2.188 | 0.07608 | 3.89 | 0.37292 |
| U105B_st | 0.724 | 0.36968 | 6.66 | 0.37351 |
| gga-miR-133a_st | 3.633 | 0.12661 | 10.82 | 0.37418 |
| HBI-43_st | 1.456 | 0.26705 | 2.34 | 0.37442 |
| hsa-miR-151-5p_st | 1.087 | 0.88294 | 3.67 | 0.37549 |
| dya-miR-133_st | 2.083 | 0.21177 | 16.94 | 0.37628 |
| bta-miR-133b_st | 2.509 | 0.04098 | 4.91 | 0.37660 |
| mmu-miR-339-5p_st | 0.804 | 0.30746 | 3.84 | 0.37916 |
| mml-miR-324-5p_st | 0.926 | 0.74992 | 365.36 | 0.37943 |
| eca-miR-376c_st | 3.733 | 0.10306 | 6.63 | 0.37943 |
| dgr-miR-133_st | 2.296 | 0.09852 | 10.37 | 0.37974 |
| eca-miR-1248_st | 1.870 | 0.40537 | 3.41 | 0.38074 |
| age-miR-128_st | 1.462 | 0.41274 | 2.34 | 0.38087 |
| ssc-miR-199a-3p_st | 1.807 | 0.23861 | 2.64 | 0.38121 |
| eca-miR-1_st | 3.245 | 0.39241 | 6.08 | 0.38121 |
| cfa-miR-10b_st | 3.919 | 0.25230 | 4.44 | 0.38223 |
| mmu-miR-324-5p_st | 1.269 | 0.51041 | 26.30 | 0.38230 |
| der-miR-133_st | 1.447 | 0.50725 | 17.89 | 0.38244 |
| dre-miR-1_st | 1.213 | 0.68797 | 2.45 | 0.38270 |
| cre-miR1164_st | 2.574 | 0.01556 | 2.10 | 0.38292 |
| spu-miR-133_st | 13.194 | 0.21079 | 52.92 | 0.38323 |
| oan-miR-1a_st | 1.953 | 0.03952 | 4.94 | 0.38324 |
| hsa-miR-133a_st | 1.672 | 0.47157 | 13.92 | 0.38352 |
| tni-miR-18_st | 2.805 | 0.01292 | 3.74 | 0.38387 |
| cfa-miR-421_st | 0.797 | 0.50935 | 2.46 | 0.38437 |
| eca-miR-18a_st | 2.521 | 0.03739 | 2.80 | 0.38472 |
| eca-miR-324-5p_st | 1.335 | 0.41181 | 11.52 | 0.38506 |
| hsa-miR-652_st | 0.716 | 0.41197 | 2.31 | 0.38553 |
| bfl-miR-1_st | 2.633 | 0.18340 | 4.26 | 0.38565 |
| spu-miR-1_st | 1.639 | 0.47467 | 5.00 | 0.38632 |
| mne-miR-10b_st | 10.229 | 0.25573 | 9.20 | 0.38706 |
| xtr-miR-133b_st | 2.567 | 0.11434 | 16.30 | 0.38733 |
| hp_rno-mir-106b_st | 1.354 | 0.13455 | 2.19 | 0.38772 |
| cin-let-7a_st | 1.368 | 0.34770 | 2.02 | 0.38816 |
| cfa-miR-30e_st | 0.755 | 0.00772 | 4.10 | 0.38887 |
| hsa-miR-454_st | 1.336 | 0.44423 | 2.50 | 0.38906 |
| lca-miR-18_st | 1.997 | 0.05534 | 2.95 | 0.39221 |
| fru-miR-135b_st | 1.174 | 0.54724 | 2.21 | 0.39228 |
| cfa-miR-652_st | 0.548 | 0.08986 | 4.88 | 0.39268 |
| ppy-miR-1_st | 2.147 | 0.19838 | 2.90 | 0.39277 |
| eca-miR-331_st | 0.779 | 0.71489 | 5.04 | 0.39403 |
| ggo-miR-133a_st | 4.346 | 0.00405 | 6.27 | 0.39443 |
| eca-miR-98_st | 1.245 | 0.44886 | 2.91 | 0.39470 |
| bta-miR-2427_st | 3.967 | 0.08922 | 2.88 | 0.39505 |
| mmu-miR-1_st | 1.989 | 0.01613 | 3.28 | 0.39540 |
| sko-miR-133_st | 1.925 | 0.04459 | 6.52 | 0.39633 |
| mml-miR-628-3p_st | 1.312 | 0.51823 | 2.60 | 0.39757 |
| bta-miR-18a_st | 3.700 | 0.01280 | 2.43 | 0.39841 |
| ppy-miR-199a-3p_st | 2.237 | 0.02035 | 2.38 | 0.39879 |
| rno-miR-98_st | 1.178 | 0.51219 | 2.83 | 0.39892 |
| ptr-miR-133a_st | 1.469 | 0.02785 | 11.18 | 0.40042 |
| tni-miR-133_st | 1.966 | 0.09259 | 25.68 | 0.40055 |
| gga-miR-133c_st | 0.852 | 0.43102 | 57.78 | 0.40060 |
| ppy-miR-489_st | 1.414 | 0.10883 | 3.62 | 0.40133 |
| oan-miR-222b_st | 2.876 | 0.04107 | 2.63 | 0.40156 |
| ppy-miR-133b_st | 2.406 | 0.37163 | 23.05 | 0.40169 |
| mmu-miR-155_st | 1.470 | 0.40408 | 2.66 | 0.40224 |
| mml-miR-146b-3p_st | 1.065 | 0.90587 | 3.53 | 0.40268 |
| sla-miR-133a_st | 1.613 | 0.35755 | 19.56 | 0.40301 |
| cte-miR-133_st | 2.727 | 0.35312 | 7.40 | 0.40333 |
| mmu-miR-30d_st | 1.329 | 0.01016 | 2.00 | 0.40335 |
| ame-miR-133_st | 1.883 | 0.03804 | 17.81 | 0.40357 |
| xtr-miR-101a_st | 0.955 | 0.90191 | 3.63 | 0.40412 |
| ppy-miR-181a-star_st | 6.154 | 0.03842 | 3.88 | 0.40472 |
| age-miR-133a_st | 2.969 | 0.01194 | 19.77 | 0.40477 |
| ssc-miR-18_st | 3.328 | 0.10583 | 2.49 | 0.40499 |
| ppy-miR-133c_st | 1.827 | 0.18409 | 10.68 | 0.40516 |
| hsa-miR-133b_st | 1.605 | 0.17647 | 9.06 | 0.40527 |
| gga-miR-454_st | 1.197 | 0.81235 | 2.86 | 0.40559 |
| ssc-miR-133a_st | 1.933 | 0.00718 | 13.07 | 0.40588 |
| tca-miR-133_st | 2.582 | 0.17085 | 17.07 | 0.40602 |
| rno-miR-133b_st | 2.059 | 0.44722 | 11.16 | 0.40602 |
| mmu-miR-106b-star_st | 3.591 | 0.00267 | 2.75 | 0.40645 |
| bta-miR-186_st | 0.618 | 0.09241 | 2.21 | 0.40648 |
| isc-miR-133_st | 2.806 | 0.21726 | 8.57 | 0.40680 |
| sla-miR-127_st | 5.049 | 0.02843 | 11.48 | 0.40680 |
| fru-miR-133_st | 1.528 | 0.29723 | 36.50 | 0.40718 |
| aae-miR-133_st | 2.612 | 0.16418 | 18.76 | 0.40742 |
| dre-miR-93_st | 1.787 | 0.02584 | 3.34 | 0.40760 |
| dre-let-7j_st | 0.723 | 0.22428 | 2.22 | 0.40767 |
| eca-miR-133b_st | 2.322 | 0.25929 | 5.32 | 0.40820 |
| hp_mmu-mir-297a-2_st | 0.762 | 0.10974 | 2.01 | 0.40822 |
| hsa-miR-199b-5p_st | 2.540 | 0.05978 | 2.69 | 0.40907 |
| rno-miR-10a-3p_st | 8.533 | 0.06265 | 3.80 | 0.41032 |
| mml-miR-489_st | 1.540 | 0.57526 | 3.98 | 0.41047 |
| dre-miR-133b_st | 1.284 | 0.26119 | 7.33 | 0.41078 |
| ppy-miR-18_st | 3.776 | 0.02544 | 2.07 | 0.41164 |
| lla-miR-133a_st | 1.183 | 0.58017 | 23.14 | 0.41215 |
| mml-miR-199a-3p_st | 2.105 | 0.04537 | 3.10 | 0.41227 |
| ACA52_st | 1.319 | 0.08787 | 2.47 | 0.41271 |
| nvi-miR-3478_st | 0.921 | 0.88570 | 3.24 | 0.41277 |
| oan-miR-194_st | 0.338 | 0.19130 | 2.35 | 0.41344 |
| ppy-miR-151-3p_st | 3.700 | 0.03659 | 3.81 | 0.41380 |
| dwi-miR-133_st | 1.797 | 0.01490 | 5.69 | 0.41418 |
| oan-miR-133b_st | 1.254 | 0.29958 | 10.53 | 0.41431 |
| mmu-miR-133a_st | 2.452 | 0.11326 | 12.38 | 0.41434 |
| bma-miR-133_st | 2.705 | 0.29032 | 8.48 | 0.41467 |
| bta-miR-98_st | 0.792 | 0.55425 | 3.00 | 0.41630 |
| cqu-miR-133_st | 0.926 | 0.56310 | 18.43 | 0.41657 |
| bta-miR-133a_st | 1.889 | 0.36547 | 21.05 | 0.41672 |
| ptr-miR-1_st | 0.964 | 0.84384 | 3.23 | 0.41732 |
| xla-miR-133b_st | 1.942 | 0.12475 | 10.05 | 0.41759 |
| eca-miR-133a_st | 2.433 | 0.33589 | 15.58 | 0.41819 |
| aae-miR-71_st | 1.376 | 0.38309 | 2.63 | 0.41823 |
| dpu-miR-133_st | 1.724 | 0.32389 | 11.87 | 0.41842 |
| cfa-miR-133c_st | 2.897 | 0.00152 | 8.12 | 0.41878 |
| mml-miR-133c_st | 2.043 | 0.24319 | 16.07 | 0.41899 |
| mml-miR-133b_st | 2.009 | 0.09125 | 31.07 | 0.41925 |
| osa-miR399j_st | 0.984 | 0.86328 | 2.55 | 0.41943 |
| hsa-miR-98_st | 1.377 | 0.16718 | 3.40 | 0.42040 |
| nvi-miR-133_st | 1.960 | 0.27766 | 13.90 | 0.42045 |
| tgu-miR-133_st | 2.176 | 0.08502 | 9.65 | 0.42049 |
| dmo-miR-133_st | 1.186 | 0.78999 | 9.15 | 0.42122 |
| mmu-miR-690_st | 0.852 | 0.72662 | 12.37 | 0.42150 |
| tgu-miR-214_st | 1.330 | 0.55128 | 3.30 | 0.42192 |
| hsa-miR-20a-star_st | 7.395 | 0.05812 | 2.50 | 0.42223 |
| cfa-miR-98_st | 1.645 | 0.06275 | 3.10 | 0.42275 |
| dse-miR-133_st | 1.946 | 0.22866 | 19.23 | 0.42280 |
| mmu-miR-24-2-star_st | 0.600 | 0.22641 | 2.24 | 0.42298 |
| dme-miR-133_st | 1.319 | 0.57213 | 12.35 | 0.42355 |
| ptr-miR-133b_st | 1.648 | 0.48866 | 9.42 | 0.42359 |
| tgu-miR-101_st | 0.934 | 0.88943 | 2.21 | 0.42443 |
| dan-miR-133_st | 1.290 | 0.68550 | 19.61 | 0.42468 |
| tni-miR-101a_st | 1.112 | 0.73889 | 2.40 | 0.42553 |
| dpe-miR-133_st | 1.366 | 0.53182 | 16.83 | 0.42570 |
| mmu-miR-30e-star_st | 0.870 | 0.76914 | 3.48 | 0.42602 |
| hsa-miR-10a-star_st | 4.196 | 0.02798 | 3.91 | 0.42764 |
| rno-miR-324-5p_st | 1.460 | 0.11778 | 7.52 | 0.42791 |
| ppa-miR-133a_st | 1.314 | 0.35886 | 14.47 | 0.42804 |
| cbr-miR-1_st | 1.481 | 0.35650 | 2.82 | 0.42827 |
| mgU2-25-61_s_st | 1.032 | 0.89309 | 2.65 | 0.42868 |
| tni-miR-1_st | 1.756 | 0.14381 | 2.25 | 0.42881 |
| ssc-miR-151-3p_st | 2.385 | 0.19129 | 3.20 | 0.42883 |
| dsi-miR-133_st | 1.762 | 0.18044 | 18.35 | 0.43011 |
| gga-miR-133b_st | 1.080 | 0.87130 | 8.73 | 0.43019 |
| mne-miR-133a_st | 2.430 | 0.07930 | 13.95 | 0.43042 |
| age-miR-28_st | 0.753 | 0.40557 | 3.30 | 0.43076 |
| ppc-miR-1_st | 1.688 | 0.25110 | 3.21 | 0.43123 |
| mml-miR-1_st | 2.260 | 0.24729 | 2.52 | 0.43128 |
| dps-miR-133_st | 1.475 | 0.06557 | 7.49 | 0.43167 |
| dre-miR-133a_st | 2.100 | 0.35445 | 9.00 | 0.43205 |
| rlcv-miR-rL1-29_st | 0.977 | 0.95996 | 2.02 | 0.43219 |
| aga-miR-133_st | 1.902 | 0.08743 | 28.30 | 0.43239 |
| oan-miR-205_st | 1.218 | 0.64638 | 3.60 | 0.43256 |
| eca-miR-374b_st | 1.889 | 0.49584 | 4.15 | 0.43301 |
| ptr-miR-331_st | 1.143 | 0.84779 | 3.61 | 0.43330 |
| bta-miR-1343-star_st | 1.153 | 0.74179 | 2.04 | 0.43376 |
| lgi-miR-1_st | 3.140 | 0.32608 | 3.99 | 0.43395 |
| hp_hsa-mir-664_s_st | 1.850 | 0.49614 | 2.55 | 0.43396 |
| ptr-miR-101_st | 1.255 | 0.43343 | 3.71 | 0.43446 |
| cfa-miR-18a_st | 3.065 | 0.06911 | 2.03 | 0.43638 |
| osa-miR818e_st | 0.781 | 0.36233 | 2.06 | 0.43658 |
| mmu-miR-199b-star_st | 2.731 | 0.06390 | 3.10 | 0.43732 |
| tni-miR-194_st | 0.425 | 0.34485 | 2.32 | 0.43850 |
| rno-miR-181a-star_st | 3.544 | 0.06880 | 2.67 | 0.43980 |
| xla-miR-133a_st | 1.404 | 0.14437 | 8.87 | 0.44116 |
| hp_mmu-mir-680-2_st | 0.856 | 0.70278 | 2.10 | 0.44196 |
| ppy-miR-376c_st | 2.855 | 0.01734 | 6.07 | 0.44240 |
| xtr-miR-133c_st | 2.694 | 0.12609 | 13.81 | 0.44265 |
| oan-miR-133a_st | 2.332 | 0.18333 | 7.58 | 0.44364 |
| bta-miR-199b_st | 2.479 | 0.21227 | 5.15 | 0.44386 |
| eca-miR-345-5p_st | 1.219 | 0.42995 | 3.35 | 0.44416 |
| ptr-miR-598_st | 1.424 | 0.22377 | 2.87 | 0.44494 |
| hsa-miR-18b_st | 4.095 | 0.09092 | 2.32 | 0.44538 |
| dre-miR-457a_st | 1.972 | 0.30658 | 2.31 | 0.44538 |
| ptr-miR-151_st | 3.289 | 0.26237 | 3.16 | 0.44682 |
| ppy-miR-1248_st | 1.772 | 0.56314 | 2.44 | 0.44733 |
| rno-miR-339-5p_st | 0.601 | 0.28854 | 9.31 | 0.44761 |
| ppa-miR-98_st | 1.023 | 0.61612 | 2.62 | 0.44786 |
| mml-miR-886-3p_st | 10.397 | 0.07258 | 5.47 | 0.44787 |
| ppt-miR2085_st | 0.907 | 0.79807 | 3.88 | 0.44795 |
| fru-let-7e_st | 1.222 | 0.44074 | 2.20 | 0.44870 |
| eca-miR-199b-3p_st | 2.352 | 0.07514 | 2.64 | 0.44881 |
| csa-miR-133_st | 3.586 | 0.14005 | 6.70 | 0.45013 |
| hsa-miR-30e-star_st | 0.801 | 0.13806 | 3.04 | 0.45029 |
| ppy-miR-551a_st | 2.810 | 0.23721 | 2.62 | 0.45058 |
| mml-miR-376c_st | 2.382 | 0.07325 | 4.13 | 0.45080 |
| cfa-miR-1843_st | 3.102 | 0.28071 | 2.94 | 0.45083 |
| hsa-miR-374a_st | 1.357 | 0.70795 | 2.43 | 0.45153 |
| hsa-miR-29c-star_st | 0.353 | 0.00335 | 2.41 | 0.45251 |
| mmu-miR-1958_st | 0.670 | 0.05026 | 5.65 | 0.45315 |
| tgu-miR-18a_st | 3.017 | 0.02277 | 2.70 | 0.45434 |
| dvi-miR-133_st | 2.357 | 0.32945 | 5.82 | 0.45443 |
| rno-miR-99b_st | 0.461 | 0.43422 | 2.67 | 0.45498 |
| cfa-miR-301a_st | 0.624 | 0.06490 | 2.25 | 0.45655 |
| hsa-miR-490-5p_st | 1.278 | 0.32470 | 2.21 | 0.45705 |
| mmu-miR-33-star_st | 1.572 | 0.17427 | 2.30 | 0.45962 |
| rno-miR-199a-3p_st | 1.749 | 0.05599 | 2.74 | 0.46110 |
| bta-miR-2440_st | 3.203 | 0.48886 | 3.70 | 0.46242 |
| bmo-miR-133_st | 2.279 | 0.25990 | 4.86 | 0.46303 |
| cfa-miR-133b_st | 2.025 | 0.17007 | 5.55 | 0.46692 |
| bta-miR-345-5p_st | 1.572 | 0.18065 | 5.13 | 0.46762 |
| mml-miR-98_st | 1.398 | 0.30438 | 2.26 | 0.46944 |
| xtr-miR-133a_st | 1.160 | 0.62692 | 4.17 | 0.46997 |
| hsa-miR-331-3p_st | 1.273 | 0.51470 | 3.16 | 0.47059 |
| ssc-miR-345-5p_st | 2.430 | 0.04969 | 2.34 | 0.47096 |
| mmu-miR-199b_st | 1.192 | 0.67283 | 2.63 | 0.47231 |
| bta-miR-199a-3p_st | 2.543 | 0.11800 | 2.62 | 0.47427 |
| xtr-miR-301_st | 0.672 | 0.01763 | 2.36 | 0.47448 |
| xtr-miR-18a_st | 3.259 | 0.02488 | 2.13 | 0.47636 |
| ptr-miR-489_st | 1.420 | 0.47941 | 2.62 | 0.47666 |
| odi-miR-1c_st | 2.264 | 0.44998 | 2.28 | 0.47915 |
| mml-miR-301a_st | 1.276 | 0.70811 | 3.12 | 0.48454 |
| hsa-miR-151-3p_st | 1.680 | 0.41209 | 2.19 | 0.48566 |
| ptr-miR-18a_st | 3.426 | 0.08141 | 2.75 | 0.48670 |
| hsa-miR-26b-star_st | 0.919 | 0.77310 | 2.06 | 0.48689 |
| rno-miR-30e-star_st | 1.109 | 0.70566 | 2.44 | 0.49077 |
| hsa-miR-376c_st | 2.731 | 0.13567 | 3.37 | 0.49384 |
| eca-miR-340-3p_st | 1.430 | 0.50561 | 2.03 | 0.49484 |
| lla-miR-28_st | 0.641 | 0.50787 | 2.29 | 0.49576 |
| ppy-miR-1201_st | 1.789 | 0.21627 | 2.54 | 0.49655 |
| ptr-miR-628_st | 1.017 | 0.95420 | 2.80 | 0.49954 |
| ppy-miR-324-3p_st | 1.855 | 0.20650 | 3.53 | 0.50173 |
| mne-miR-181a-star_st | 5.963 | 0.06913 | 2.44 | 0.50331 |
| ssc-miR-339_st | 0.701 | 0.21092 | 3.57 | 0.50382 |
| mmu-miR-28_st | 0.885 | 0.79473 | 2.53 | 0.50423 |
| mdo-miR-140_st | 0.593 | 0.08266 | 2.01 | 0.51323 |
| rno-miR-331_st | 1.388 | 0.51352 | 2.10 | 0.52029 |
| tni-miR-181a-star_st | 2.644 | 0.04098 | 2.53 | 0.52484 |
| bta-miR-199c_st | 3.148 | 0.04036 | 2.34 | 0.52872 |
| cfa-miR-101_st | 1.399 | 0.60707 | 2.45 | 0.52970 |
| gga-miR-199-star_st | 1.478 | 0.29925 | 2.59 | 0.53127 |
| hsa-miR-628-3p_st | 0.615 | 0.33215 | 2.11 | 0.53461 |
| tgu-miR-301_st | 0.829 | 0.62656 | 2.17 | 0.53704 |
| oan-miR-301_st | 0.745 | 0.24932 | 2.09 | 0.53782 |
| dre-miR-146a_st | 0.925 | 0.85478 | 2.04 | 0.54102 |
| tgu-miR-140_st | 0.387 | 0.02194 | 2.38 | 0.54135 |
| hsa-miR-221-star_st | 3.039 | 0.04024 | 2.34 | 0.54486 |
| ptr-miR-199b_st | 1.897 | 0.16315 | 2.66 | 0.54584 |
| oan-miR-199_st | 2.568 | 0.13218 | 2.01 | 0.54791 |
| eca-miR-151-5p_st | 2.304 | 0.22882 | 2.36 | 0.54879 |
| ppy-miR-324-5p_st | 1.256 | 0.25120 | 3.65 | 0.54951 |
| mmu-miR-1839-5p_st | 1.198 | 0.73955 | 2.16 | 0.55060 |
| bta-miR-151-star_st | 0.989 | 0.98245 | 2.56 | 0.55389 |
| osa-miR1318_st | 0.819 | 0.44521 | 2.17 | 0.55485 |
| bta-miR-331_st | 1.873 | 0.33604 | 2.43 | 0.55543 |
| bta-miR-454_st | 1.713 | 0.42340 | 2.03 | 0.55663 |
| mml-miR-152_st | 0.282 | 0.22825 | 2.25 | 0.56263 |
| ppa-miR-214_st | 0.942 | 0.18148 | 2.16 | 0.57470 |
| mml-miR-598_st | 1.149 | 0.14220 | 2.28 | 0.57873 |
| hsa-miR-766_st | 3.815 | 0.03904 | 2.77 | 0.61432 |
| dre-miR-214_st | 1.113 | 0.69400 | 2.09 | 0.61839 |
| ssc-miR-331-3p_st | 1.276 | 0.52197 | 2.10 | 0.62039 |
| bta-miR-2395_st | 2.650 | 0.47798 | 2.12 | 0.62298 |
| mmu-miR-331-3p_st | 0.624 | 0.03994 | 2.06 | 0.62789 |
| bta-miR-339b_st | 0.797 | 0.26496 | 2.10 | 0.62996 |
| ssc-miR-324_st | 2.013 | 0.29057 | 2.21 | 0.67343 |

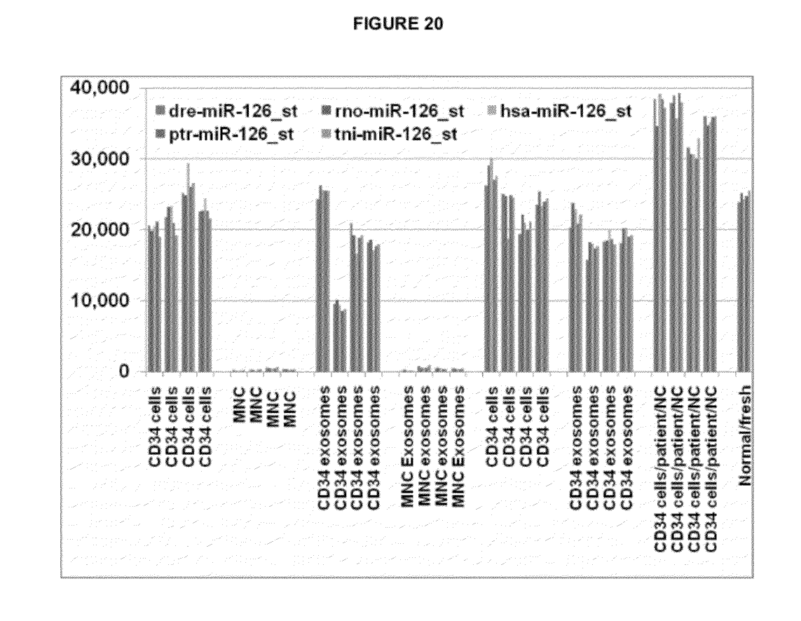

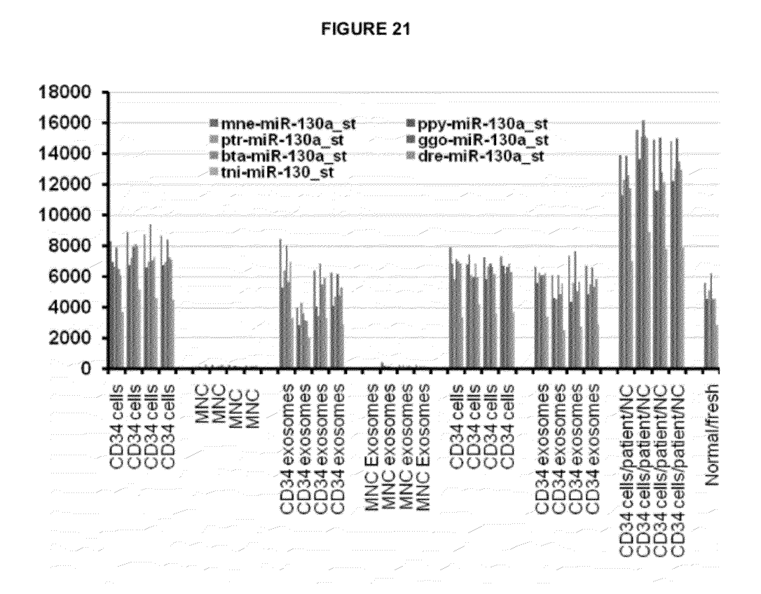

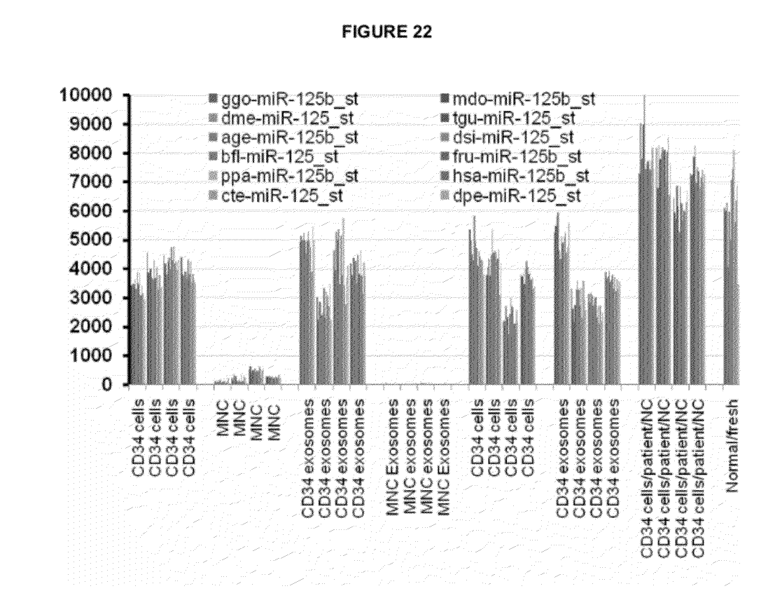

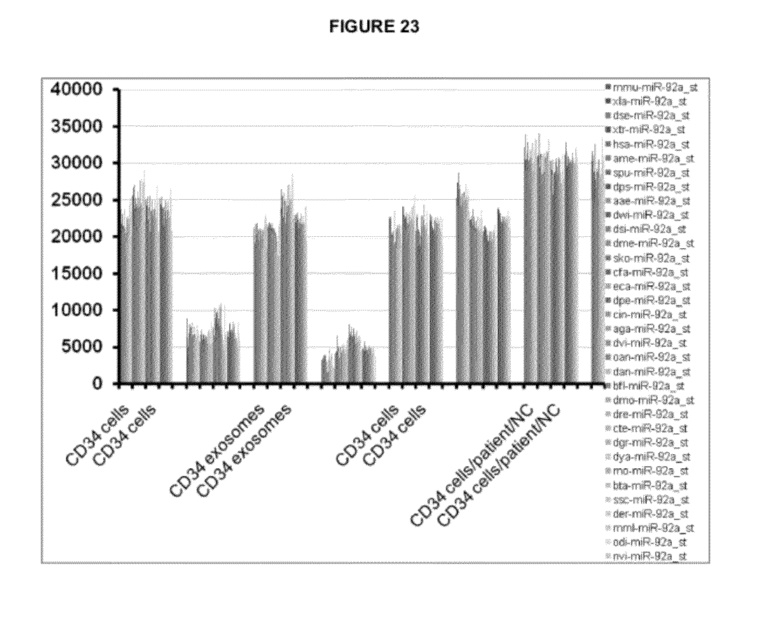

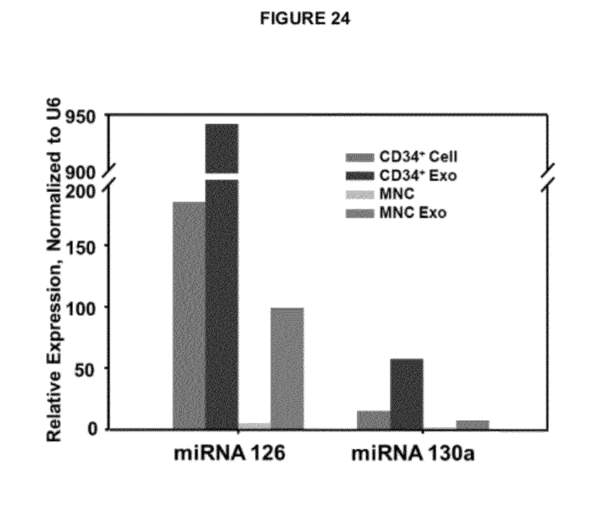

These data show that the expression of several well-known pro-angiogenic miRNAs (see, e.g., Bonauer et al, Curr Drug Targets, 2010, 11: 943; Urbich et al, Cardiovasc Res, 2008, 79(4): 5818) such as miRNA 126 and miRNA 130a was about 80-fold and 50-fold higher in CD34+ cells (p=0.04) and exosomes (p=0.07), respectively, compared with MNCs and MNC exosomes (FIG. 20, 21; Table 2). For instance, expression of miRNA 130a (see, e.g., Zhang, Q. et al. Biochem Biophys Res Commun. 2011, 405:42-46) was about 50-fold higher in both CD34+ cells (p=0.008) and CD34+ exosomes (p=0.04) relative to the MNC cells and exosomes (FIG. 21; Table 2). Expression of miRNA 125b was about 14-fold higher in CD34+ cells (p=0.008) and about 180-fold higher in CD34+ exosomes (p=0.01) compared to the MNC counterparts (FIG. 22, Table 2). miRNA 92a was about 5-fold higher in both the CD34+ cells (p=0.0005) and exosomes (p=0.0008) relative to MNCs and MNC exosomes (FIG. 23, Table 2). Higher expression was detected in CD34+ exosomes as compared to MNC exosomes for miRNA 126 and miRNA 130a, which had approximately 9-fold and 10-fold higher expression in CD34+ exosomes (FIG. 24) similar to their cellular expressions. The miRNA microarray data was validated by qRT-PCR Taqman miRNA expression assays for the miRNAs demonstrating the most different expression. Overall, these data show that CD34+ exosomes are enriched for several pro-angiogenic miRNAs.

### Example 6

### Transfer of CD34+ Exosome to HUVECs

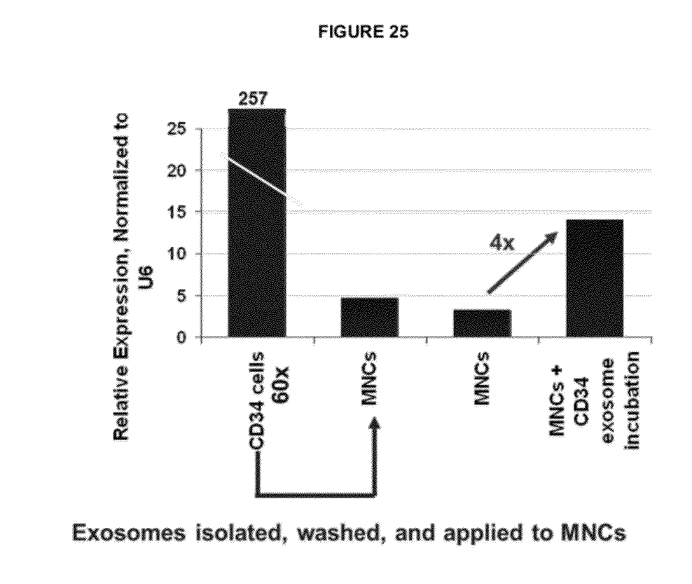

During the development of embodiments of the technology provided herein, experiments demonstrated that the amount of miRNA 126 in MNCs (which has about 50-fold lesser expression as compared to CD34+ cells) increased 4-fold after incubation with CD34+ exosomes (FIG. 25) compared to the untreated control. These data demonstrate the transfer of miRNA from the exosomes to cells.

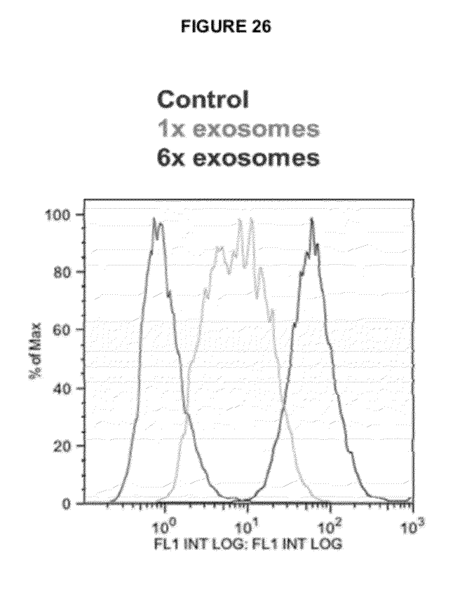

Live imaging by confocal microscopy demonstrated the uptake of DiI labeled CD34+ exosomes by HUVECs following a 20-minute incubation of the HUVECs with the exosomes. This uptake of CD34+ exosomes by HUVECS is concentration dependent, as shown by flow cytometry analysis of HUVECs incubated with a 6× concentration of exosomes, which resulted in a higher intensity of DiI (FIG. 26). These results demonstrate that adult human CD34+ derived exosomes carry and transfer pro-angiogenic miRNA to recipient cells.

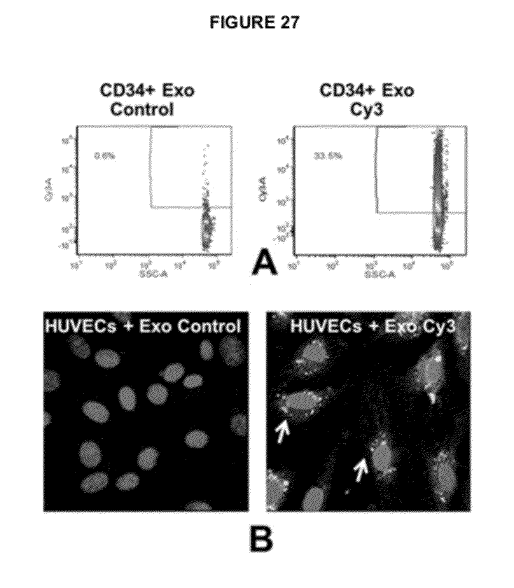

In additional experiments, it was demonstrated that Cy3-labeled miRNA is secreted from CD34+ cells. CD34+ cells were transfected with Cy3-labeled miRNA using lipofectamine reverse-transcription method. Either only lipofectamine or only Cy3 miRNA treatment without lipofectamine was taken as control. Flow cytometry analysis of the cells indicated successful transfection. Isolated intact exosomes were RNAse-treated and then tagged to 4-μm latex beads for flow cytometry analysis. The data indicated that the Cy3 is released via exosomes (FIG. 27a). Data also indicated the presence of Cy3 miRNA in the intracellular punctate vesicles of HUVECs, thus demonstrating that CD34+ exosomes transfer Cy3-labeled miRNA to HUVECs. Live imaging by confocal microscopy was used to acquire images of CD34+ cells transfected with Cy3 miRNA and to monitor the uptake of Cy3 into the cytosol of HUVECs. Multiple confocal images were acquired.

These data show that the exosomes secreted by CD34+ cells were morphologically similar in size and shape to exosomes described in previous reports, carried known exosomal protein markers, and induced angiogenic activity both in vitro and in vivo. Furthermore, the exosomes were sufficiently durable to remain intact and biologically active throughout the isolation procedure, which suggests that the functional radius of CD34+ exosomes could extend beyond the immediate vicinity of the secreting cell. Without being bound by any particular theory, the observation that exosomes from CD34+ cells were more potent than the cells themselves may indicate that the exosomes' superior durability may provide the ability to deliver a high dose of exosomes via collection from culture medium in which exosomes are secreted over a period of time. However, an understanding of the mechanism is not needed to practice the technology described herein, nor is the technology limited by any particular mechanism of action.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1) A method comprising administering to a subject a therapeutically effective amount of purified adult stem cell vesicles or adult stem cell vesicle extract.

2) The method of claim 1 wherein the vesicles are exosomes.

3) The method of claim 1 wherein the vesicles contain TSG101 and CD63 proteins.

4) The method of claim 1 wherein the vesicles contain CD34+ protein.

5) The method of claim 1 wherein the vesicles are exosomes prepared from CD34+ cells.

6) The method of claim 1 wherein the stem cells are isolated from cord blood, bone marrow, peripheral blood, brain, spinal cord, blood vessels, skeletal muscle, skin, teeth, heart, gut, liver, ovarian epithelium, amniotic fluid, umbilical cord, or testis.

7) The method of claim 1 wherein the vesicles are delivered by injection catheter, by intramyocardial injection, by intracoronary administration, by intracoronary infusion, by an intravenous injection, or nanoparticles.

8) The method of claim 1 wherein the subject requires angiogenic therapy.

9) The method of claim 1 wherein the subject has a disease selected from the group consisting of cardiovascular disease, infarction, chronic wounds, ulcer, clogged vessels, damaged vessels, stenotic vessels, atherosclerosis, angina, peripheral vascular disease, critical limb ischemia, ischemic heart disease, hypoxic tissues, heart failure, bone marrow disease, Alzheimer's disease, diabetes, and Parkinson's disease.

10) The method of claim 1 wherein the subject requires wound healing, scar reduction, or tissue regeneration.

11) The method of claim 1 wherein the subject has a bone marrow transplant.

12) The method of claim 1 wherein the subject has tissue damage from a stroke, hemorrhage, thrombosis, embolism, or hypoperfusion.

13) A composition comprising purified and isolated adult stem cell vesicles or adult stem cell vesicle extract.

14) The composition of claim 13 comprising a therapeutic amount of the purified and isolated adult stem cell vesicles or the adult stem cell vesicle extract.

15) The composition of claim 13 comprising at least 10⁴ vesicles.

16) The composition of claim 13 wherein the vesicles are exosomes.

17) The composition of claim 13 wherein the vesicles are from at least 10⁴ stem cells or wherein the extract is from at least 10⁴ stem cells.

18) The composition of claim 13 wherein an amount of the vesicles is at least 0.1 gram.

19) The composition of claim 13 essentially free of non-vesicle stem cell components.

20) The composition of claim 13 wherein the vesicles are cup shaped, are 30-100 nm in diameter, or have a density of 1.1-1.2 g/ml.

21) The composition of claim 13 wherein the vesicles contain TSG101 and CD63 proteins.

22) The composition of claim 13 wherein the vesicles contain CD34+ protein.

23) The composition of claim 13 wherein the vesicles are prepared from CD34+ cells.

24) The composition of claim 13 wherein the vesicles are derived from an autologous source.

25) The composition of claim 13 wherein the vesicles are derived from an allogeneic source.

26) The composition of claim 13 wherein the vesicles are derived from an autologous source by a method comprising:
a) mobilizing CD34+ cells by treating the autologous source with a mobilizing agent;
b) enriching the CD34+ cells using apheresis; and
c) further enriching the CD34+ cells using a magnetic bead cell selection device.

27) The composition of claim 26 in which the mobilizing agent is GCSF or AMD3100.

28) A method of preparing vesicles comprising culturing adult CD34+ stem cells in conditioned media, isolating the cells from the conditioned media, and purifying the vesicles to generate a purified preparation of adult CD34+ stem cell vesicles.

29) The method of claim 28 wherein the CD34+ cells are derived from a GCSF-mobilized or AMD3100-mobilized source of animal adult stem cells.

30) The method of claim 28 wherein the source of animal adult stem cells is peripheral blood.

31) The method of claim 28 wherein the conditioned media is supplemented with 0.1-5% human serum albumin, FLT ligand, SCF, and VEGF.

32) The method of claim 28 wherein purifying comprises sequential centrifugation.

33) The method of claim 28 further comprising clarifying the vesicles on a density gradient.

34) The method of claim 28 further comprising freezing the vesicles.
